(19) United States
(12) Patent Application Publication
(10) Pub. No.: US 2026/0002156 A1
(43) Pub. Date: Jan. 1, 2026

(54) MODULAR RNA DELIVERY PLATFORMS AND METHODS OF THEIR USE

(71) Applicant: ARIZONA BOARD OF REGENTS ON BEHALF OF ARIZONA STATE UNIVERSITY, Scottsdale, AZ (US)

(72) Inventors: Hao YAN, Gilbert, AZ (US); Yang XU, Chandler, AZ (US); Xiaodong QI, Chandler, AZ (US); Lu YU, Tempe, AZ (US); Liangxiao CHEN, Tempe, AZ (US)

(21) Appl. No.: 19/120,697

(22) PCT Filed: Oct. 13, 2023
(86) PCT No.: PCT/US2023/076899

§ 371 Date: Apr. 11, 2025

Related U.S. Application Data
(60) Provisional application No. 63/416,285, filed on Oct. 14, 2022.

(51) Int. Cl. C12N 15/113; A61P 35/00; C07K 14/47; C12N 15/88

(52) U.S. Cl. CPC C12N 15/113; A61P 35/00; C07K 14/47; C12N 15/88; C12N 2310/14; C12N 2310/531

(57) ABSTRACT

Described herein are mRNA carrier systems and methods of their use for entering the cytoplasm and expressing carried mRNA, to induce a tumor killing effect. The mRNA carrier systems can comprise DNA or RNA nanostructures. Also described herein are methods of treating cancer using the mRNA carrier systems described herein.

### RELATED APPLICATIONS

This disclosure claims priority to U.S. Provisional Application No. 63/416,285, filed Oct. 14, 2022, the contents of which are herein incorporated by reference in their entirety.

### FIELD

The present invention relates to compositions and methods for treating patients using modular RNA (e.g., small interfering RNA (siRNA), small hairpin RNA (shRNA), and/or mRNA) delivery platforms. The treatment can be based on immunological (e.g., vaccine delivery) and/or gene knockdown (e.g., siRNA, shRNA) approaches. The delivery platforms can include DNA or RNA nanostructures.

### SEQUENCE STATEMENT

The instant application contains a Sequence Listing, which has been submitted electronically and is hereby incorporated by reference in its entirety. Said file, is named G8118-03601_SEQ_ID_LISTING.xml, created on Oct. 6, 2023, and is 1,713,153 bytes in size.

### INCORPORATION BY REFERENCE

Throughout this application, various publications are referenced. All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### BACKGROUND

The following includes information that may be useful in understanding the present invention. It is not an admission that any of the information, publications or documents specifically or implicitly referenced herein is prior art, or essential, to the presently described or claimed inventions. All publications, patents, related applications, and other written or electronic materials mentioned or identified herein are hereby incorporated herein by reference in their entirety. The information incorporated is as much a part of the application as filed as if all of the text and other content was repeated in the application, and should be treated as part of the text and content of the application as filed.

Single stranded RNA (ssRNA) and double stranded RNA (dsRNA) can be detected by pattern recognition receptors in mammalian cells. For example, polyinosinic: polycytidylic acid (polyIC), a synthetic analog of dsRNA, has been widely studied as an adjuvant in treating diseases such as upper respiratory tract infections and tumors, therefore, allowing it to be explored as an adjuvant in flu and cancer vaccines. However, susceptibility of dsRNA to nuclease digestion tends to be a concern especially when they are used in vivo.

RNA interference (RNAi) is a highly conserved posttranscriptional gene silencing mechanism mediated by small double-stranded RNA molecules. Small interfering RNA (siRNA) or based RNAi therapeutics holds the promise to treat various human diseases most of which are genetic disorders that lack conventional therapies. Most RNAi therapeutics (e.g., patisiran, givosiran, and lumasiran), including several clinical trials, have been focused on the treatment of liver diseases. Messenger RNA (mRNA) delivery is an important component in vaccines against infectious disease.

### SUMMARY

The inventions described and claimed herein have many attributes and embodiments including, but not limited to, those set forth or described or referenced in this Brief Summary. It is not intended to be all-inclusive and the inventions described and claimed herein are not limited to or by the features or embodiments identified in this introduction, which is included for purposes of illustration only and not restriction.

In certain aspects, this disclosure provides for RNA sequence (siRNA, shRNA, and/or mRNA) carrier platforms for rapid and targeted delivery of said RNA sequences into cytoplasm of tumor cells and/or immune cells of both innate and adaptive immune systems, which induces a direct tumor killing effect and/or tumor killing effect triggered by the immune system programmed by the RNA sequence delivery platform. Described herein is a nucleic acid (DNA or RNA) nanodevice used to carry small interfering RNA (siRNA), small hairpin RNA (shRNA), or programmed mRNAs (Pro-mRNA) therapeutics to targeted areas. The nucleic acid nanodevices are effective at targeting tumor cells and/or immune cells specifically and safely to deliver therapeutics to the tumor site. The nucleic acid nanodevice comprises a siRNA, shRNA, or Pro-mRNA, and acts as a carrier to deliver said RNA through rapid and targeted siRNA, shRNA, or Pro-mRNA delivery into the cytoplasm of tumor cells and/or immune cells.

In certain aspects, this disclosure provides for a multivalent RNA oligonucleotide junction delivery complex comprising:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the second RNA oligonucleotide to form a first arm, and also partially complementary to the fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects, the first, second, third, and fourth peripheral oligonucleotides each independently comprise DNA, RNA, or both.

In some aspects, the enhancer sequence initiates the Pro-mRNA sequence translation.

In some aspects, the enhancer sequence comprises a G-gap independent Internal ribosome entry site (IRES) which can enhance initiation of Pro-mRNA translation. In some aspects, the enhancer sequence comprises a recruiter to a RNA binding protein sequence, wherein the RNA binding protein comprises the eukaryotic initiation factor 3 (elF3) complex. In some aspects, the enhancer and Pro-mRNA sequence are contiguous.

In some aspects, the four core RNA oligonucleotides arms of the complex form a cross-shape with each arm about at a 90 degree angle to two neighboring arms.

In some aspects, the targeting molecule is selected from: GalNAc, an affibody, shRNA, or anti-cancer agent. The affibody can be an antibody or portion thereof to ASPH or Her2. In some aspects, the targeting molecule can be GalNAc. In some aspects, the shRNA can include or exclude MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13).

In some aspects, the complex comprises two Pro-mRNA contiguous sequences with RNA protein binding sites and enhancers, two disulfide-modified peripheral oligonucleotides, and two targeting molecules.

In some aspects, the Pro-mRNA sequence encodes for a cancer neoantigen. In some aspects, the cancer neoantigen is expressed from all or a portion of a tumor associated gene, in particular a tumor associate gene selected from: AP2S1, Survivin, CTSL, MPZL2, and LSP1. In some aspects, the Pro-mRNA sequence encodes for a portion or all of a viral surface protein. In some aspects, the viral surface protein is a surface protein from SARS-COV-2. In some aspects, the SARS-COV-2 surface protein is a Spike protein(S).

In some aspects, the Pro-mRNA sequence comprises a Kozak sequence. The Kozak sequence can comprise the sequence AUG. In some aspects, the Pro-mRNA sequence comprises a polyA tail having from 25 to 35 contiguous adenosines. In some aspects, the Pro-mRNA sequence comprises an open reading frame (ORF).

In some aspects, the Pro-mRNA sequence comprises a long mRNA strand sequence and a plurality of RNA staple strands. In some aspects, the portions of the long mRNA strand are partially complementary to each of the plurality of RNA staple strands such that the Pro-mRNA folds into a selected secondary structure. In some aspects, the secondary structure of the Pro-mRNA is selected from a flat sheet, a tube, a tetrahedron, or a square box. The flat sheet can be in the shape of a square, rectangle, triangle, about circule, or hexagon.

In some aspects, the Pro-mRNA sequence is about 5,000 to 15,000 bases in length.

In some aspects, the Pro-mRNA sequence can further comprise a plurality of staple strands each of which comprises a unique sequence and is hybridized to a specific bindable ssRNA region on the Pro-mRNA sequence.

In some aspects, the targeting molecule can be configured to be at pre-defined positions on the first, second, third, or fourth peripheral oligonucleotides.

In some aspects, the mRNA strand of the Pro-mRNA complex comprises a T7 promoter sequence (5′-TAATACGACTCACTATAG-3′) (SEQ ID NO:188). In some aspects, the mRNA strand of the Pro-mRNA complex comprises a 5′ G cap. The 5′ G cap can include or exclude: CleanCap AG™, CleanCap GG™, CleanCap AU™, CleanCap AG Ome™, CleanCap AU Ome™ (Trilink Technologies, Inc.), ARCA, 5′ m7G capm, or 5′ m7G cap. In some embodiments, the 5′ G cap can be selected from the caps described in U.S. Pat. Nos. 10,563,195, or 10913768, or U.S. Patent Application No. 63/267,223 (also published as WO2023147352), each of which is herein incorporated by reference.

In some aspects, this disclosure provides for a pharmaceutical composition comprising the complex as described herin and a pharmaceutically acceptable carrier. The composition can further comprise an anti-cancer agent, including the anti-cancer agents described herein.

In some aspects, this disclosure provides for a shRNA sequence selected from: MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13). In some aspects, this disclose provides for a lipid nanoparticle comprising a shRNA sequence selected from: MCL-1 shRNA, MCL-12 shRNA, MCL-34 shRNA, BCL-XL shR12, BCL-XL shR13, or BCL-XL shR34.

In some aspects, this disclosure provides for a method of killing a cancer cell, the method comprising presenting a shRNA sequence selected from MCL-1 shRNA, MCL-12 shRNA, MCL-34 shRNA, BCL-XL shR12, BCL-XL shR13, or BCL-XL shR34, or a multivalent RNA oligonucleotide junction delivery complex to a cancer cell, wherein the complex comprises:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the
second RNA oligonucleotide to form a first arm, and also partially complementary to the
fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects, this disclosure relates to a method of treating cancer in a subject in need thereof, the method comprising administering to the subject a therapeutically effective amount of a composition comprising a lipid nanoparticle comprising a shRNA selected from: MCL-1 shRNA, MCL-12 shRNA, MCL-34 shRNA, BCL-XL shR12, BCL-XL shR13, or BCL-XL shR34, or a multivalent RNA oligonucleotide junction delivery complex to a subject, wherein the complex comprises:

a. four core RNA oligonucleotides comprising:

a first RNA oligonucleotide,
a second RNA oligonucleotide,
a third RNA oligonucleotide, and
a fourth RNA oligonucleotide,
where the a portion of the first RNA oligonucleotide is partially complementary to the
second RNA oligonucleotide to form a first arm, and also partially complementary to the
fourth RNA oligonucleotide to form a fourth arm;

where a portion of the second RNA oligonucleotide is partially complementary to the
third RNA oligonucleotide to form a second arm, and is also partially complementary to the first RNA oligonucleotide to form the first arm;

where a portion of the third RNA oligonucleotide is partially complementary to the fourth RNA oligonucleotide to form the third arm and also partially complementary to the second RNA oligonucleotide to form the second arm;

and wherein a portion of the fourth RNA oligonucleotide is partially complementary to
the first RNA oligonucleotide to form the fourth arm, and is partially complementary to
the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects of the methods, the cancer is a colorectal cancer, pancreatic cancer, breast cancer, ovarian cancer, lung cancer or melanoma.

In some aspects, the method further comprises administering to the subject a therapeutically effective amount of at least one anti-cancer agent.

The use of a multivalent RNA oligonucleotide junction delivery complex in the preparation of a medicament for the treatment of cancer,
wherein the complex comprises:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the second RNA oligonucleotide to form a first arm, and also partially complementary to the fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects of the use, the cancer is selected from a colorectal cancer, pancreatic cancer, breast cancer, ovarian cancer, lung cancer or melanoma.

In some aspects, this disclosure provides for a lipid nanoparticle comprising a lipid and a multivalent RNA oligonucleotide junction delivery complex to a cancer cell, wherein the complex comprises:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the second RNA oligonucleotide to form a first arm, and also partially complementary to the fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects, this disclosure provides for a method of inducing an immune response in a subject, the method comprising administering to the subject a multivalent RNA oligonucleotide junction delivery complex to a cancer cell, wherein the complex comprises:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the second RNA oligonucleotide to form a first arm, and also partially complementary to the fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects, this disclosure provides for a RNA oligonucleotide carrier comprising:
a. a nucleic acid nanodevice comprising a single sheet of double stranded DNA or RNA (dsDNA or dsRNA) which comprises at least four edges of which at least two opposite edges independently comprise a single stranded DNA or RNA region, and further comprising a first surface and a second surface;
b. a targeting antibody configured to be on the first surface, which is optionally anti-Aspartate beta-hydroxylase; and
c. a locking peptide conjugated at both the C-and N-terminus independently with a peptide-linked single-stranded oligonucleotide, wherein at least a portion of each said peptide-linked single-stranded oligonucleotide is complementary to at least a portion of one of said single stranded DNA or RNA regions,
wherein the first and second surfaces of the dsDNA or dsRNA each independently further comprise one or a plurality of bindable single-stranded DNA or RNA (ssDNA or ssRNA) regions.

In some aspects, the linear disulfide (LD)-modified siRNA comprises: 5 repeated LD units introduced at the 5′ end of the passenger strand and the 3′ ends of the guide strand. In some aspects, the small RNA oligonucleotide carrier does not result in RNA-induced silencing complex (RISC) formation because of the introduction of the LD unit to the 5′ end of the guide strand.

In some aspects, the single dsDNA or dsRNA sheet can be of about 5,000 to 10,000 bases in length.

In some aspects, the dsDNA or dsRNA can further comprise a plurality of staple strands each of which comprises a unique sequence and is hybridized to a specific bindable ssDNA or ssRNA region.

In some aspects, the dsDNA or dsRNA can further comprises one or more fastener strands of DNA, wherein the one or more fastener strands of DNA or RNA is capable of fastening the sheet into an nanostructure. In some aspects, the nanostructure is a tube. In some aspects, the nanostructure is a sheet. In some aspects, the nanostructure is DNA or RNA origami.

In some aspects, the one or a plurality of bindable single-stranded DNA or RNA (ssDNA or ssRNA) regions can be operably linked to a targeting moiety selected from an antibody to a cancer cell surface-expressing protein, a lipid bilayer anchor, a siRNA sequence modified with a sulfide modifier, Programmed mRNA (Pro-mRNA), short hairpin RNA shRNA sequence (shRNA), or combinations thereof. The disulfide modifier can be a penta-disulfide modifier.

The operable linkage can be through conjugation through the same oligonucleotide sequence such that the targeting moiety is conjugated to the bindable ssDNA or ssRNA. The operable linkage can be through hybridization of the bindable ssDNA or ssRNA to a complement oligonucleotide sequence which is conjugated to the targeting moiety. The RNA oligonucleotide carrier can comprise at least two bindable ssDNA or ssRNA regions, wherein a first bindable ssDNA or ssRNA region is configured to be on a first surface and comprises an antibody to a cancer cell surface-expressing protein or a lipid bilayer anchor, and a second bindable ssDNA or ssRNA region configured to be on a second surface and comprises a siRNA sequence modified with a sulfide modifier or a shRNA sequence. In some aspects, there are two shRNA sequences. The two shRNA sequences can be selected from MCL-1 shRNA, MCL-12 shRNA, MCL-34 shRNA, BCL-XL shR12, BCL-XL shR13, or BCL-XL shR34.

In some aspects, the lipid membrane anchor can be selected from cholesterol, a fatty acid alcohol, or a fatty acid ether. In some aspects, the fatty acid alcohol is selected from decanol, dodecanol, tetradecanol, or octadecanol. In some aspects, the lipid membrane anchor is chemically conjugated through a modified alcohol group to the targeting oligonucleotide.

In some aspects, the locking peptide is an ADAM10 substrate.

In some aspects, the nucleic acid nanodevice is in a tubular shape.

In some aspects, the targeting moieties is configured to be at pre-defined positions on the single sheet of dsDNA or dsRNA.

In some aspects, this disclosure provides for a pharmaceutical composition comprising the RNA oligonucleotide carrier as described herin and a pharmaceutically acceptable carrier. The composition can further comprise an anti-cancer agent, including the anti-cancer agents described herein.

In some aspects, this disclosure provides for a method of presenting shRNA or siRNA into a cell, the method comprising:
a. contacting a cell comprising an ADAM10 surface protein and an ASPH surface protein with a RNA oligonucleotide carrier wherein the carrier is in a tubular shape,
b. cleaving the locking peptide with the ADAM10 surface protein,
c. opening the RNA oligonucleotide carrier from a tubular shape to a semi-flat shape, and
d. presenting disulfuide-modified siRNA, and/or shRNA, into the cell, wherein the RNA oligonucleotide carrier comprises:
   i. a nucleic acid nanodevice comprising a single sheet of double stranded DNA or RNA (dsDNA or dsRNA) which comprises at least four edges of which at least two opposite edges independently comprise a single stranded DNA or RNA region, and further comprising a first surface and a second surface;
   ii. a targeting antibody configured to be on the first surface, which is optionally anti-Aspartate beta-hydroxylase; and
   iii. a locking peptide conjugated at both the C- and N-terminus independently with a peptide-linked single-stranded oligonucleotide, wherein at least a portion of each said peptide-linked single-stranded oligonucleotide is complementary to at least a portion of one of said single stranded DNA or RNA regions, wherein the first and second surfaces of the dsDNA or dsRNA each independently further comprise one or a plurality of bindable single-stranded DNA or RNA (ssDNA or ssRNA) regions.

In some aspects, this disclosure provides for a RNA oligonucleotide carrier comprising:
a. a single-sheet of a single sheet of double stranded DNA or RNA (dsDNA or dsRNA) which comprises a first surface and a second surface;
b. one or a plurality of cancer cell-targeting antibodies configured to be on the first surface of said single sheet of dsDNA or dsRNA;
c. one or a plurality of lipid membrane anchors configured to be on the second surface of said single sheet of dsDNA or dsRNA, wherein a membrane anchor is in contact with a lipid nanoparticle or cell,
wherein the lipid nanoparticle or cell comprises a RNA oligonucleotide and/or an anti-cancer agent, and
wherein the first and second surfaces of the dsDNA or dsRNA each independently further comprise one or a plurality of bindable single-stranded DNA or RNA (ssDNA or ssRNA) regions.

The one or a plurality of bindable single-stranded DNA or RNA (ssDNA or ssRNA) regions can be operably linked to said lipid membrane anchor, or to said targeting antibody, or both. The targeting antobidy can be an antibody to ASPH. The lipid membrane anchor can be selected from cholesterol, fatty acid alcohol, or fatty acid ether. The fatty acid alcohol can be selected from: decanol, dodecanol, tetradecanol, or octadecanol.

In some aspects, the RNA oligonucleotide is within a smart RNA oligonucleotide complex as described herein.

In aspects, this disclosure provides for a smart RNA oligonucleotide complex comprising:
a. a carrier molecule;
b. a first heterodouble shRNA molecule which comprises 2 loops region and a trigger oligonucleotide can be complementary to at least a portion of sites at the 3′-ends of said shRNA molecule;
c. a second heterodouble shRNA molecule which 2 loops region and a trigger oligonucleotide can be complementary to at least a portion of sites at the 5′-ends of said shRNA molecule; and
d. one or a plurality of conditional logic RNA molecules which release in the presence of a trigger event.

In some aspects, the shRNA molecules can connect to the conditional logic RNA molecules, and connect to the carrier molecule. The conditional logic RNA molecules can be antisense RNA molecules against tumor cell specific mRNAs. The tumor cell specific mRNA can be ASPH mRNA.

In some aspects, this disclosure provides for a RNA oligonucleotide carrier which is a multivalent siRNA oligonucleotide junction delivery complex comprising: three, four, or six RNA oligonucleotides which form three, four, or six arms, repectively; wherein each of the arms are a Dicer enzyme substrate. In some aspects, each of the arms can further comprise a peripheral end, and wherein said peripheral end comprises two non-complementary strands such that at least a portion of the arm comprises two separate single stranded RNA sequences. The two separate single stranded RNA sequences together can hybridize to a trigger RNA sequence, wherein the trigger RNA sequence can be a complement of a portion of an mRNA sequence in a cancer cell, optionally an ASPH mRNA sequence. The ASPH gene, for which the mRNA is based, can be found at: chr8: 61,500,556-61,714,640 (GRCh38/hg38), Size: 214,085 bases, Orientation: Minus strand. In some aspects, the ASPH mRNA sequence is selected from: NM_001164750.2, NM_001164751.2, NM_001164752.2, NM_001164753.2, NM_001164754.2, NM_001164755.2, NM_001164756.2, NM_004318.4, NM_020164.5, NM_032466.4, NM_032467.4, and NM_032468.5, the sequences of which are well-known in the art and available at: www.genecards.org/cgi-bin/carddisp.pl?gene=ASPH). In some aspects, the trigger RNA sequence is modified with polyethylene glycol (PEG), a lipid, and/or a targeting molecule. In aspects, the targeting molecule is an shRNA, antibody or portion thereof to ASPH (ASPH-Aspartyl/asparaginyl beta-hydroxylase). In some aspects, the targeting molecule is GalNAc. In some aspects, the shRNA can include or exclude MCL-1 shRNA, MCL-12 shRNA, MCL-34 shRNA, BCL-XL shR12, BCL-XL shR13, or BCL-XL shR34

In some aspects, this disclosure provides for a RNA oligonucleotide carrier which is a multivalent siRNA oligonucleotide junction delivery complex comprising: three, four, or six RNA oligonucleotides which form three, four, or six arms, repectively; wherein each of the arms is a Dicer enzyme substrate. In some aspects, each of the arms can further comprise a peripheral end, and wherein said peripheral end comprises two non-complementary strands such that at least a portion of the arm comprises two separate single stranded RNA sequences. The two separate single stranded RNA sequences together can hybridize to a trigger RNA sequence, wherein the trigger RNA sequence can be a complement of a portion of an mRNA sequence in a cancer cell, optionally an ASPH mRNA sequence. In aspects, the trigger RNA sequence is modified with polyethylene glycol (PEG), a lipid, and/or a targeting molecule. In aspects, the targeting molecule can be an antibody or portion thereof to ASPH. In aspects, the targeting molecule is GalNAc.

The Dicer enzyme cleaves double-stranded RNA (dsRNA) and pre-microRNA (pre-miRNA) into short double-stranded RNA fragments referred to as small interfering RNA and microRNA, respectively. These aforementioned fragments are about 20-25 base pairs long and comprise a two-base overhang on the 3′-end.

In some aspects, this disclosure provides for a programmed mRNA (Pro-mRNA) complex comprising:
a. a mRNA strand; and
b. a plurality of RNA staple strands,
wherein the mRNA is folded into a configuration with the RNA staple strands into a compact RNA nanostructure comprising a plurality of helixes.

In some aspects, the mRNA strand of the Pro-mRNA complex comprises a T7 promoter sequence (5′-TAATACGACTCACTATAG-3′) (SEQ ID NO:188). In some aspects, the mRNA strand of the Pro-mRNA complex comprises a 5′ G cap. The 5′ G cap can include or exclude: CleanCap AG™, CleanCap GG™, CleanCap AU™, CleanCap AG Ome™, CleanCap AU Ome™ (Trilink Technologies, Inc.), ARCA, 5′ m7G capm, or 5′ m7G cap. In some embodiments, the 5′ G cap is selected from the caps described in U.S. Pat. Nos. 10,563,195, or 10913768, or U.S. Patent Application No. 63/267,223 (also published as WO2023147352), each of which is herein incorporated by reference.

In some aspects, the RNA nanostructure of the Pro-mRNA complex is a shape having a 6-helix bundle.

In some aspects, the Pro-mRNA complex is resistant to nuclease degradation.

In some aspects, the mRNA strand of the Pro-mRNA complex comprises a 5′ untranslated region (UTR) and a 3′ UTR. In some aspects, the 5′ UTR and the 3′ UTR are spatially separated in the Pro-mRNA complex. In some aspects, the 5′ UTR and the 3′ UTR are spatially substantially adjacent to each other.

In some aspects, the mRNA strand of the Pro-mRNA complex is circularized.

In some aspects, the mRNA of the Pro-mRNA complex encodes for a modified infectious disease surface protein or therapeutic proteins or surface receptor proteins of immune cells of both innate and adaptive immune systems for programmed in-vivo cell engineering and in-vivo cell therapy (in vivo NK cell therapy and/or in vivo T cell therapy).

In some aspects, the mRNA strand of the Pro-mRNA complex encodes for a immunogenic neoantigen protein.

In some aspects, this disclosure provides for a method of inducing an immune response in a subject, the method comprising administering to the subject the Pro-mRNA complex comprising a mRNA strand and a plurality of RNA staple strands, wherein the mRNA is folded into a configuration with the RNA staple strands into a compact RNA nanostructure comprising a plurality of helixes, wherein the Pro-mRNA complex enters a cell comprising cytosol, and the mRNA strand then dissociates from the RNA staple strands in the cytosol.

In some aspects, the Pro-mRNA complex is part of a multivalent RNA oligonucleotide junction delivery complex comprising:
a. four core RNA oligonucleotides comprising:
a first RNA oligonucleotide,
a second RNA oligonucleotide,
a third RNA oligonucleotide, and
a fourth RNA oligonucleotide,
where the a portion of the first RNA oligonucleotide is partially complementary to the second RNA oligonucleotide to form a first arm, and also partially complementary to the fourth RNA oligonucleotide to form a fourth arm;
where a portion of the second RNA oligonucleotide is partially complementary to the third RNA oligonucleotide to form a second arm, and is also partially complementary to the first RNA oligonucleotide to form the first arm;
where a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
a first peripheral oligonucleotide;
a second peripheral oligonucleotide;
a third peripheral oligonucleotide; and
a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides;
wherein the trigger is selected from a disulfide-modified sequence, a targeting molecule, an enhancer sequence, a programmed mRNA (Pro-mRNA) sequence, or combinations thereof.

In some aspects, the enhancer and Pro-mRNA sequences are contiguous.

In some aspects, the targeting molecule is selected from: GalNAc, an affibody, or anti-cancer agent. The affibody can be an affibody to Her2 or ASPH.

In some aspects, there are two Pro-mRNA contiguous with RNA protein binding sites and enhancers, two disulfide-modified peripheral oligonucleotides, and two targeting molecules.

In some aspects, the Pro-mRNA encodes for a cancer neoantigen. The cancer neoantigen can be expressed from a tumor associated gene, which can include or exclude a gene selected from: AP2S1, Survivin, CTSL, MPZL2, and LSP1.

In some aspects, the Pro-mRNA comprises a Kozak sequence. The Kozak sequence can comprises the sequence AUG. In some aspects, the Pro-mRNA can comprise a polyA tail having from 25 to 35 contiguous adenosines. In certain aspects, the polyA tail is 30 contiguous adenosine bases.

In some aspects, the Pro-mRNA comprises an open reading frame (ORF). The the Pro-mRNA can comprises a long mRNA strand and a plurality of RNA staple strands. The portions of the long mRNA strand are partially complementary to each of the plurality of RNA staple strands such that the Pro-mRNA folds into a selected shape. The shape can be selected from a 3-D tube, sheet, triangle, or hexagon, or any of the DNA or RNA origami shapes described herein.

The invention also provides processes disclosed herein that are useful for preparing the RNA oligonucleotide carrier systems as described herein or a composition as described herein.

In some aspects, this disclosure provides for a method of killing a cancer cell, the method comprising presenting a RNA oligonucleotide carrier or complex described herein to a cancer cell. The cancer cell can be selected from a colorectal cancer, pancreatic cancer, breast cancer, ovarian cancer, lung cancer or melanoma.

In some aspects, this disclosure provides for a method of treating cancer in a subject in need thereof, the method comprising administering to the subject a therapeutically effective amount of a composition comprising a RNA oligonucleotide carrier or complex as described herein to a subject. The cancer can be a colorectal cancer, pancreatic cancer, breast cancer, ovarian cancer, lung cancer or melanoma. In some aspects, the method can further comprise administering to the subject a therapeutically effective amount of at least one anti-cancer agent.

In some aspects, this disclosure provides a kit comprising a pharmaceutical composition as described herein and an additional anti-cancer agent.

### BRIEF DESCRIPTION OF DRAWINGS

The following detailed description, given by way of example, but not intended to limit the invention solely to the specific embodiments described, may best be understood in conjunction with the accompanying drawings.

FIG. 1A depicts a schematic of the one embodiment of a Nucleic acid nanodevice designed to target ASPH protein, controlled open upon peptide lock cleavage by ADAM10, and cytosolic internalization via thiol-mediated uptake. The shRNAs trigger the RNAi for tumor cell apoptosis.

FIG. 1B depicts a schematic for a representative embodiment of the components of a representative Nucleic acid nanodevice for targeted shRNA delivery. The Nucleic acid nanodevice comprises a scaffold DNA or RNA strand, staple strands, and is further functionalized with a targeting moiety, e.g., an ASPH-targeting moiety (which can include or exclude an ASPH-antibody, aptamer, or binding portion thereof), a disulfide-conjugated DNA or RNA, and one or a plurality of shRNA sequences (which can include or exclude MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13), and an enzyme substrate, e.g., an ADAM10 cleavable peptide (which can include or exclude an ADAM10 peptidase substrate).

FIG. 2A shows gene silencing by representative hetero-double shRNAs of this disclosure, in particular knockdown of the Mcl-1 protein by hetero-double shRNA. Hep3B cells were transfected with the indicated hetero-double shRNA. After shRNA transfection, cells were lysed and subject to Western blot assay.

FIG. 2B shows the knockdown of the anti-apoptotic Bcl-xl protein by the indicated pools of siRNA.

FIG. 2C shows the effects of the combinatorial siRNA knockdown of Mcl-1 and Bcl-xl protein on the colonies of Hep3B cells.

FIG. 3 shows the results of a fluorogenicpeptide cleavage assay. Substrate peptide (Mca-PLAQAV-Dpa-RSSR) exhibits stronger fluorescence upon incubation with HCC cancer cells, comparing with PBS negative control or a recombinant human ADAM10 protein.

FIG. 4 shows the cellular uptake of a representative nucleic acid nanostructure of this disclosure. Confocal images of HeLa cells incubated with FAM-ADO, open FAM-5-DADO, and closed FAM-5-DADO for 1 hour. Red color (darker grey) represents lysotracker dye. Scale bar: 25 microns.

FIG. 5 is a schematic of LNP loaded with targeting molecules. The cholesterol modified DNA oligonucleotides are loaded onto the LNP surface through the fusion of cholesterol and lipid. The targeting molecule is conjugated to complimentary DNA or RNA oligonucleotides which hybridize to the cholesterol modified nucleic acid, directing LNP to specific target cells.

FIG. 6A shows a scheme and supporting data for a representative Programmable DNA or RNA nanostructure platform of this disclosure for targeted delivery of LNP. In particular, FIG. 6A. is a schematic of a DNA or RNA nanostructure design with targeting antibodies loaded on one surface of the nanostructure and cholesterol modification on a second surface of the nanostructure.

FIG. 6B. is an AFM image of the DNA nanostructure. The scale bar is 200 nm.

FIG. 6C. is a schematic of the LNP with DNA or RNA nanostructure platform for specific delivery to target cells.

FIG. 6D is confocal images of SUVs attached with FAM labeled DNA nanostructures modified with and without cholesterol. Scale bar: 50 μm.

FIG. 7A is a schematic of a representative embodiment of an intelligent shRNA delivery platform of this disclosure.

FIG. 7B shows the knockdown of the anti-apoptotic Bcl-xl protein by the indicated pools of siRNA. The top image is an agarose gel image of N/P determination of shRNA-liposome nanoparticle (Western Blot in HepG2 cells for 48h). The liposome compositions were prepared with N/P ratio of, 3 and 5 and were compared with lipofectamine. The bottom chart is the histography of Mcl-1 protein expression.

FIG. 8A is a schematic of multivalent siRNAs. In particular, FIG. 8A depicts a siRNAs design with 3, 4, or 6 RNA molecules connected to form three-way, four-way, and six-way junctions, respectively.

FIG. 8B is a schematic drawing of smart release design. Trigger RNA molecules hybridize with the four-way junction siRNA design which protect the free ends and provide selective release of siRNA molecules. Modification with PEG and/or lipid on the trigger RNA further enhances the cellular stability, while targeting molecule conjugation (e.g. GalNAc, antibody, small molecule drug) enables delivery specificity.

FIG. 8C is a schematic drawing of one embodiment of the smart release design. A multivalent programmable messenger RNA target delivery of mRNA molecules is depicted wherein the complex comprises RNA oligonucleotides, enhancer sequences to promote nanostructured mRNA translation, and disulfide modified oligonucleotides to promote cytostolic internalization.

FIG. 9 is a schematic of siRNA-coupled pRNA nanostrutcure. A mixture of mRNA, short RNA staples and RNA staples containing siRNA sequence can assemble into a programmable RNA nanostructure. The mRNA can express targeting protein (bottom left), and the siRNA can form RISC to digest target gene. The siRNA strands present at the surface of the hexagonally packed helic of double-stranded mRNA and stables strands, and are partially unhybridized.

FIG. 10 shows confocal microscopy images tracking the cellular uptake and intracellular distribution of representative disulfide modified DNA nanostructures of this disclosure. The Left panel is the schematic drawing of DNA nanostructures with or without disulfide modification. The Right panel is the confocal images of HeLa cells that incubated with DNA nanostructure in 2 hr. The FAM-DNA nanostructure panel shows the presence of DNA nanostructures; The lysotracker panel shows endosome or lysosome; the Hoescht panel shows the cell nucleus. The merged panel shows that the DNA nanostructures surrounds the nucleus after cellular uptake. Scale bar equals 25 microns.

FIGS. 11A and 11B show DNA nanostructures loading capacity and stability characterization. In particular, FIG. 11A shows fluorimeter analysis of the relationship between relative fluorescence intensity and the number of DNA on each DNA nanostructure.

FIG. 11B is a gel electrophoresis-based nuclease resistance study of 104 6-disulfide modified DNA nanostructure in HeLa complete medium (with 10% FBS) at 37° C. with varied incubation times.

FIG. 12A and FIG. 12B show characterization results of representative tubular-shaped DNA nanodevices of this disclosure. FIG. 12A is an Agarose gel electrophoresis image of DNA nanotube with 85 shRNA capture strands and 104 disulfide modified DNA capture strands (left), DNA nanotube loaded with 85 Mcl-1 shRNA (middle) and DNA nanotube loaded with 85 Mcl-1 shRNA plus 104 disulfide modified DNA (right). FIG. 12B shows representative AFM images of structure (left) and (right). Scale bar, 200 nm.

FIGS. 13A and 13B show experiments indicating that disulfide modification increases the protein inhibition effect of shRNA loaded on DNA nanostructures. In particular FIG. 13A is an Agarose gel electrophoresis image of DNA nanostructures (DON) with different modifications. FIG. 13B is a western blot (WB) analysis of Mcl-1 protein expression in HeLa cells for 48h. Both open DNA nanostructures with and without disulfide modification are loaded with 85 Mcl-1 shRNA.

FIGS. 14A-14C show an overview of RNA transcription. FIG. 14A depicts a schematic of T7 polymerase transcription. FIG. 14B is a gel image showing that double stranded PCR products can be made by using the designed T7/T7 term primer of this disclosure. FIG. 14C is a gel image showing that transcription of RNA occurs by a unique yield yield T7 polymerase.

FIGS. 15A-15D show an overview of RNA-protein translation in vitro. FIG. 15A is a schematic of internation Ribosome entry site (IRES) mediated protein expression. FIG. 15B is a black/white photograph of UV-irradiated green fluorescent protein (GFP) produced by the methods described herein. FIG. 15C shows the GFP fluorophore value was measured by a fluorophore plate reader and found to be significantly higher in expression when the IRES promotor (“P”) sequence was used to express the GFP. FIG. 15D shows the GFP protein expression levels are validated by SDS-PAGE gel.

FIGS. 16A and 16B show the characterization of representative lipid nanoparticles (LNP) of this disclosure. FIG. 16A shows TEM images and sizing information. FIG. 16B shows nanoparticle tracking analysis (NTA) of three separate batches of LNPs, showing the mean size of formed LPNs was consistently about 100 nm.

FIG. 17 shows the concept of using nanostructured mRNA with the use of staple strands to protect against RNA degradation resulting in higher translation levels in the expressed protein.

FIG. 18 shows the design of one embodiment of nanostructured RNA (“Pro-mRNA”) where more stable mRNA results in a higher amount of mRNA presented to the cell, thereby increasing the efficiency of translation for mRNA. The mRNA molecule is designed with three fragments: an internal ribosome entry site (IRES) for the cap-free translation initiation, an open reading frame for the translation of the target protein, (e.g. green fluorescence protein (GFP)), and a 3′ polyA tail for mRNA stability. The mRNA strand and 31 short DNA staple strands are designed into a rectangular-shape origami by Cadnano, with 11 bp as the helix pitch.

FIG. 19 are confocal images demonstrating the folded mRNA (mRNA pOG, origami) can be translated into GFP in the cell. The control is the same treatment without any mRNA or mRNAOG.

FIG. 20A is an agarose gel electrophoresis image showing that the nanostructured mRNA (Pro-mRNA, or mRNA OG) is formed with staple strands and has a molecular weight higher than mRNA alone or the staple strands alone.

FIG. 20B is a TEM image showing that the nanostructured mRNA (Pro-mRNA, or mRNA OG) forms structured nanosheets. Not shown is a TEM of mRNA without staple strands which does not form nanosheets.

FIG. 20C shows the stability of nanostructured mRNA (Pro-mRNA, or mRNA OG) over time, compared to mRNA without staple strands which exhibit significant degradation.

### DETAILED DESCRIPTION

The entire document is intended to be related as a unified disclosure, and it should be understood that all combinations of features described herein are contemplated, even if the combination of features are not found together in the same sentence, or paragraph, or section of this document. The present disclosure as illustratively described in the following may suitably be practiced in the absence of any element or elements, limitation or limitations, not specifically disclosed herein.

### Certain Definitions

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure.

Units, prefixes, and symbols are denoted in their System International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety. As used herein, the term “about” means±10%.

As used herein, the term “administering” refers to the physical introduction of an agent to a subject, using any of the various methods and delivery systems known to those skilled in the art. Exemplary routes of administration for the formulations disclosed herein include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. In some embodiments, the formulation is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

As used herein, the term “epitope” is defined as the part(s) of an antigen molecule which contact the antigen binding site of an antibody or a T cell receptor.

As used herein, the term “operably linked” or “operably connected” refers to the association two chemical moieties so that the function of one is affected by the other, e.g., an arrangement of elements wherein the components so described are configured so as to perform their usual function.

The term “nucleic acid” refers to deoxyribonucleotides or ribonucleotides and polymers thereof in either single- or double-stranded form, made of monomers (nucleotides) containing a sugar, phosphate and a base that is either a purine or pyrimidine. Unless specifically limited, the term encompasses nucleic acids containing synthetic analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, and the sequence explicitly indicated. Specifically, degenerate codon substitutions may be achieved by generating sequences in which the third position of one or more selected (or all) codons is substituted with mixed-base and/or deoxyinosine residues.

In some embodiments, the nucleotide can be a noncanonical base. The noncanonical base can be selected from 1-methylpseudouridine, pseudouridine, m5C, m5U, m6A, s2U, and 2′-O-methyl-U. In some embodiments, the noncanonical base can be substituted for uridine to modify the mRNA immunogenicity. In some embodiments, the modified nucleoside is at least one of 1-methyl-pseudouridine and m5C. In some embodiments, the modified nucleoside is at least one of pseudouridine and m5C. In some embodiments, the noncanonical base can be a base described in U.S. Pat. Nos. 11,060,107; 8,748,089; 9,012,219; 11,141,478; 9,181,319; and U.S. Patent Application Publication No. US20190225644; each of which is herein incorporated by reference.

The term “enhancer” as used herein refers to a sequence which can increase ethe translation efficiency (and thus yield) of an mRNA sequence into a protein. In some embodiments, the enhancer sequence is the Internal Ribosome Entry site (SEQ ID NO: 18). In some embodiments, the enhancer sequence can be one found in U.S. Patent No. U.S. Pat. No. 4,670,388, US U.S. Pat. No. 8,785,611, U.S. Patent Application Publication No. US20180171340, or Pfeiffer, B. et al. PNAS, April 9, (2012) 109 (17) 6626-6631, herein incorporated by reference. In some embodiments, the messenger RNA with an enhancer sequence exhibits enhanced ability to be translated by a target cell than a messenger RNA with the same sequence. In some embodiments, the messenger RNA with enhancer exhibits enhanced ability to be translated when delivered to a mammal than an unpurified preparation of messenger RNA with the same sequence.

The terms “nucleotide sequence”, “polynucleic acid”, or “nucleic acid sequence” refer to a sequence of bases (purines and/or pyrimidines) in a polymer of DNA or RNA, which can be single-stranded or double-stranded, optionally containing synthetic, non-natural or altered nucleotide bases capable of incorporation into DNA or RNA polymers, and/or backbone modifications (e.g., a modified oligomer, such as a morpholino oligomer, phosphorodiamate morpholino oligomer or vivo-mopholino). The terms “oligo”, “oligonucleotide” and “oligomer” may be used interchangeably and refer to such sequences of purines and/or pyrimidines. The terms “modified oligos”, “modified oligonucleotides”, “modified oligomers”, “modified ribonucleosides” or “modified ribonucleotides” may be similarly used interchangeably, and refer to such sequences that contain synthetic, non-natural or altered bases and/or backbone modifications. In some embodiments, the modified oligos can comprise chemical modifications to the internucleotide phosphate linkages and/or to the backbone sugar.

Modified nucleic acids (e.g., staple strands chemically connected or conjugated to a targeting molecule) need not be uniformly modified along the entire length of the molecule. Different nucleotide modifications and/or backbone structures may exist at various positions in the nucleic acid. The nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, including at least 50% modified nucleotides, at least 80% modified nucleotides, or at least 90% modified nucleotides.

The modified oligonucleotides (RNA and/or DNA) described herein can be synthesized and/or modified by methods well established in the art, including those described in “Current Protocols in Nucleic Acid Chemistry,” Beaucage, S. L. et al. (Edrs.), John Wiley & Sons, Inc., New York, N.Y., USA, which is hereby incorporated herein by reference in its entirety. Transcription methods are described further herein in the Examples.

In some embodiments, a staple strand is a modified nucleic acid.

The synthetic modified oligonucleotides described herein include modifications to prevent rapid degradation by endo- and exo-nucleases and to avoid or reduce the cell's innate immune or interferon response to the oligonucleotide. Modifications can include or exclude, for example, (a) end modifications, e.g., 5′ end modifications (phosphorylation dephosphorylation, conjugation, inverted linkages, etc.), 3′ end modifications (conjugation, DNA nucleotides, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, as well as (d) internucleoside linkage modifications, including modification or replacement of the phosphodiester linkages.

Modified nucleotides can include or exclude: alkylated purines and/or pyrimidines; acylated purines and/or pyrimidines; or other heterocycles. These classes of pyrimidines and purines can include or exclude: pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Backbone modifications are can include or exclude: chemical modifications to the phosphate linkage (e.g., phosphorodiamidate, phosphorothioate (PS), N3′phosphoramidate (NP), boranophosphate, 2′,5′phosphodiester, amide-linked, phosphonoacetate (PACE), morpholino, peptide nucleic acid (PNA) and inverted linkages (5′-5′ and 3′-3′ linkages)) and sugar modifications (e.g., 2′-O-Me, UNA, LNA).

The oligonucleotides described herein may be synthesized using standard solid or solution phase synthesis methods. In certain embodiments, the oligonucleotides are synthesized using solid-phase phosphoramidite chemistry (U.S. Pat. No. 6,773,885) with automated synthesizers. Chemical synthesis of nucleic acids allows for the production of various forms of the nucleic acids with modified linkages, chimeric compositions, and nonstandard bases or modifying groups attached in chosen places through the nucleic acid's entire length.

As used herein, when describing two polynucleotides (also referred to as “polynucleotide sequence”) as “operably linked”, a single-stranded or double-stranded nucleic acid moiety comprises the two polynucleotides arranged within the nucleic acid moiety in such a manner that at least one of the two polynucleotides is able to exert a physiological effect by which it is characterized upon the other. In some embodiments, a promoter polynucleotide sequence operably linked to the coding region of a gene promotes transcription of the coding region.

As used herein, when describing an oligonucleotide as “connected” to another moiety, the oligonucleotide is covalently bound to said moiety. The covalent bond is formed using a linker molecule (e.g., sulfo-SMCC (ThermoFisher), where the oligonucleotide is 5′ or 3′ modified with the appropriate functional group (amino, sulfuryl, carboxyl, aldehyde, etc.-all available from Glen Research Inc.) to bind to the linker. The moiety can likewise be functionalized, or possess an innate functional group (e.g., amino group from a lysine amino acid in a protein) to react to said linker group.

When the nucleic acid encoding the desired protein further comprises a promoter/regulatory sequence, the promoter/regulatory is positioned at the 5′ end of the desired protein coding sequence such that it drives expression of the desired protein in a cell.

Conventional notation is used herein to describe polynucleotide sequences: the left-hand end of a single-stranded polynucleotide sequence is the 5′-end; the left-hand direction of a double-stranded polynucleotide sequence is referred to as the 5′-direction.

The direction of 5′ to 3′ addition of nucleotides to nascent RNA transcripts is referred to as the transcription direction. The DNA strand having the same sequence as an mRNA is referred to as the “coding strand”.

As used herein, the term “promoter sequence” refers to a nucleic acid sequence which is required for expression of a gene product operably linked to the promoter/regulatory sequence. In some embodiments, this conjoined sequence may be the core promoter sequence. In other embodiments, this conjoined sequence may also comprise an enhancer sequence and other regulatory elements which are required for expression of the gene product. In some embodiments, the promoter/regulatory sequence may be one which expresses the gene product in a tissue specific manner.

Certain embodiments of the invention encompass isolated or substantially purified nucleic acid compositions. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule is substantially free of other cellular material or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived.

By “portion” or “fragment,” as it relates to a nucleic acid molecule, sequence or segment of the invention, when it is linked to other sequences for expression, is meant a sequence having at least 80 nucleotides, at least 150 nucleotides, or at least 400 nucleotides. If not employed for expressing, a “portion” or “fragment” means at least 9, at least 12, at least 15, or at least 20, consecutive nucleotides, e.g., probes and primers (oligonucleotides), corresponding to the nucleotide sequence of the nucleic acid molecules of the invention.

As used herein, the term “recombinant” refers to a combination of DNA or RNA sequences that are joined together using recombinant DNA or RNA technology and procedures used to join together DNA or RNA sequences as described, for example, in Sambrook and Russell, Molecular Cloning: A Laboratory Manual, Cold Spring Harbor, NY: Cold Spring Harbor Laboratory Press (3rd edition, 2001). In some embodiments, recombinant nucleic acids are the the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions and may act to modulate production of a desired product by various mechanisms. Alternatively, DNA sequences encoding RNA that is not translated may also be considered recombinant. Recombination may also be achieved by either chemical synthesis means, or by the artificial manipulation of isolated segments of nucleic acids, e.g., by genetic engineering techniques.

“Homology” refers to the percent identity between two polynucleotides or two polypeptide sequences. Two DNA or polypeptide sequences are “homologous” to each other when the sequences exhibit at least about 75% to 85% (including 75%, 76%, 77%, 78%, 79%, 80%, 81%, 82%, 83%, 84%, and 85%), at least about 90%, or at least about 95% to 99% (including 95%, 96%, 97%, 98%, 99%) contiguous sequence identity over a defined length of the sequences.

As described herein, RNA nanostructures comprising at least one ssRNA molecule (e.g., one or more oligonucleotides/polynucleotides) may be prepared using methods described herein, as well as, with respect to certain embodiments, using techniques known in the art. The assembly of such RNA nanostructures may be based on base-pairing principles or other non-canonical binding interactions. For example, while no specific RNA sequence is required, regions of complementary within a single RNA molecule or between multiple RNA molecules may be used for assembly. Persons of ordinary skill in the art will readily understand and appreciate that the optimal sequence for any given RNA nanostructure will depend on the desired or intended shape, size, nucleic acid content, and intended use of such RNA structure. In certain embodiments, wherein the nanostructure comprises more than one ssRNA molecule (e.g. two or more oligonucleotides/polynucleotides), each ssRNA molecule may have a region that is complementary to a region on another ssRNA molecule to enable hybridization of the strands and assembly of the nanostructure. In certain other embodiments, wherein the nanostructure consists of a single ssRNA molecule (i.e., a single unimolecular RNA oligonucleotide/polynucleotide), regions within the molecule may be complementary to certain other regions within the molecule to enable hybridization and assembly of the nanostructure.

RNA nanostructures produced in accordance with the present disclosure are typically nanometer-scale structures (e.g., having length scale of 1 to 1000 nanometers), although, in some instances, the term “nanostructure” herein may refer to micrometer-scale structures (e.g., assembled from more than one nanometer-scale or micrometer-scale structure). In some embodiments, a RNA nanostructure described herein has a length scale of 1 to 1000 nm, 1 to 900 nm, 1 to 800 nm, 1 to 700 nm, 1 to 600 nm, 1 to 500 nm, 1 to 400 nm, 1 to 300 nm, 1 to 200 nm, 1 to 100 nm or 1 to 50 nm. In some embodiments, a RNA nanostructure described herein has a length scale of greater than 1000 nm. In some embodiments, a RNA nanostructure described herein has a length scale of 1 micrometer to 2 micrometers.

In certain embodiments, the RNA nanostructure comprises, consists essentially of, or consists of multiple ssRNA molecules (e.g., more than one oligonucleotide/polynucleotide strands, such as two or more ssRNA molecules). In certain embodiments, the RNA nanostructure comprises two or more ssRNA molecules, which are capable of self-assembling (or configured to self-assemble) into a nanostructure. In certain embodiments, the RNA nanostructure is assembled from two or more ssRNA molecules through paranemic cohesion crossovers. Thus, in certain embodiments, the RNA nanostructure comprises two or more ssRNA molecules, wherein the ssRNA molecules self-assemble to form at least one paranemic cohesion crossover.

In certain embodiments, the RNA nanostructure comprises, consists essentially of, or consists of a single ssRNA molecule (i.e., one unimolecular oligonucleotide/polynucleotide strand). In certain embodiments, the RNA nanostructure is assembled using one ssRNA molecule (e.g., in certain embodiments one and only one, exactly one, or greater than zero and less than two). In certain embodiments, the RNA nanostructure is comprised of one ssRNA molecule, which is capable of self-assembling into a nanostructure. In certain embodiments, the RNA nanostructure consists of one ssRNA molecule, which is capable of self-assembling into a nanostructure. In certain embodiments, the RNA nanostructure is assembled from one ssRNA molecule through paranemic cohesion crossovers. Thus, in certain embodiments, the RNA nanostructure comprises one single-stranded RNA (ssRNA) molecule, wherein the ssRNA molecule forms at least one paranemic cohesion crossover.

The length of each RNA strand within an RNA nanostructure is variable and depends on, for example, the type, size, geometric, and/or intended use of nanostructure to be formed. It is to be understood, that if a particular RNA nanostructure comprises more than one ssRNA molecule, the length of each RNA molecule can be selected independently of one another. In certain embodiments, the at least one ssRNA molecule (i.e., oligonucleotide or RNA strand) is about 10 nucleotides in length to about 200,000 nucleotides in length, the at least one ssRNA molecule (i.e., oligonucleotide or RNA strand) is about 10 nucleotides in length to about 100,000 nucleotides in length, the at least one ssRNA molecule (i.e., oligonucleotide or RNA strand) is about 10 nucleotides in length to about 90,000 nucleotides in length, about 10 to about 80,000 nucleotides in length, about 10 to about 70,000 nucleotides in length, about 10 to about 60,000 nucleotides in length, about 10 to about 50,000 nucleotides in length, about 10 to about 40,000 nucleotides in length, about 10 to about 30,000 nucleotides in length, about 10 to about 25,000 nucleotides in length, or about 10 to about 20,000 nucleotides in length. In certain embodiments, the at least one ssRNA molecule (i.e., oligonucleotide or RNA strand) is about 15 nucleotides in length to about 20,000 nucleotides in length, the ssRNA molecule (i.e., oligonucleotide or RNA strand) is about 15 nucleotides in length to about 10,000 nucleotides in length, about 15 to about 7500 nucleotides in length, about 3000 to about 7000 nucleotides in length, about 5000 to about 7000 nucleotides in length, about 1500 to about 6500 nucleotides in length, about 1000 to about 7000 nucleotides in length, about 5500 to about 6500 nucleotides in length, about 15 to about 5000 nucleotides in length, about 15 to about 4000 nucleotides in length, about 15 to about 3000 nucleotides in length, about 250 to about 3000 nucleotides in length, about 500 to about 3000 nucleotides in length, about 1000 to about 3000 nucleotides in length, or about 1500 to about 2500 nucleotides in length. In certain embodiments, the ssRNA molecule (i.e., oligonucleotide or RNA strand) is about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, about 10000, about 10100, about 10200. about 10300, about 10400, about 10500, about 10600, about 10700, about 10800, about 10900, about 11000, about 11000, about 11100, about 11200, about 11300, about 11400, about 11500, about 11600, about 11700, about 11800, about 11900, about 12000, about 12100, about 12200, about 12300, about 12400, about 12500, about 12600, about 12700, about 12800, about 12900 nucleotides in length, about 13000 nucleotides in length, about 14000 nucleotides in length, about 15000 nucleotides in length, about 16000 nucleotides in length, about 17000 nucleotides in length, about 18000 nucleotides in length, about 19000 nucleotides in length, about 20000 nucleotides in length, about 25000 nucleotides in length, about 30000 nucleotides in length, about 35000 nucleotides in length, about 40000 nucleotides in length, about 45000 nucleotides in length, about 50000 nucleotides in length, about 75000 nucleotides in length, about 100000 nucleotides in length, about 125000 nucleotides in length, about 150000 nucleotides in length, about 175000 nucleotides in length or about 200000 nucleotides in length.

In certain embodiments, an ssRNA molecule used in an RNA nanostructure described herein is synthesized de novo using any number of procedures well known in the art. For example, the cyanoethyl phosphoramidite method (Beaucage, S. L., and Caruthers, M. H., Tet. Let. 22:1859,1981) or the nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622,1988). These chemistries can be performed by a variety of automated oligonucleotide synthesizers available in the market, including the use of an in vitro transcription method.

An ssRNA molecule used in an RNA nanostructure described herein may comprise one or more modifications. Such modifications include, but are not limited to, base modifications, sugar modifications, and backbone modifications. The ssRNA molecule may contain natural or synthetic nucleotides (e.g., modified nucleotides). For example, in certain embodiments, the ssRNA nanostructure comprises one or more modified nucleotides (e.g., one or more inosine residues). ssRNA molecules described herein may have a homogenous backbone (e.g., entirely phosphodiester or entirely phosphorothioate) or a heterogeneous (or chimeric) backbone.

Modified nucleotides are known in the art and include, by example and not by way of limitation, alkylated purines and/or pyrimidines; acylated purines and/or pyrimidines; or other heterocycles. These classes of pyrimidines and purines are known in the art and include, pseudoisocytosine; N4, N4-ethanocytosine; 8-hydroxy-N6-methyladenine; 4-acetylcytosine, 5-(carboxyhydroxylmethyl) uracil; 5-fluorouracil; 5-bromouracil; 5-carboxymethylaminomethyl-2-thiouracil; 5-carboxymethylaminomethyl uracil; dihydrouracil; inosine; N6-isopentyl-adenine; 1-methyladenine; 1-methylpseudouracil; 1-methylguanine; 2,2-dimethylguanine; 2-methyladenine; 2-methylguanine; 3-methylcytosine; 5-methylcytosine; N6-methyladenine; 7-methylguanine; 5-methylaminomethyl uracil; 5-methoxy amino methyl-2-thiouracil; β-D-mannosylqueosine; 5-methoxycarbonylmethyluracil; 5-methoxyuracil; 2-methylthio-N6-isopentenyladenine; uracil-5-oxyacetic acid methyl ester; psueouracil; 2-thiocytosine; 5-methyl-2 thiouracil, 2-thiouracil; 4-thiouracil; 5-methyluracil; N-uracil-5-oxyacetic acid methylester; uracil 5-oxyacetic acid; queosine; 2-thiocytosine; 5-propyluracil; 5-propylcytosine; 5-ethyluracil; 5-ethylcytosine; 5-butyluracil; 5-pentyluracil; 5-pentylcytosine; and 2,6,-diaminopurine; methylpsuedouracil; 1-methylguanine; 1-methylcytosine. Backbone modifications are similarly known in the art, and include, chemical modifications to the phosphate linkage (e.g., phosphorodiamidate, phosphorothioate (PS), N3′phosphoramidate (NP), boranophosphate, 2′,5′phosphodiester, amide-linked, phosphonoacetate (PACE), morpholino, peptide nucleic acid (PNA) and inverted linkages (5′-5′ and 3′-3′ linkages)) and sugar modifications (e.g., 2′-O-Me, UNA, LNA).

In certain embodiments, the at least one ssRNA molecule does not comprise a transcription termination sequence (e.g., in the middle of the strand). In certain embodiments, the at least one ssRNA molecule does not comprise an AUCUGUU sequence.

In certain embodiments, an RNA nanostructure described herein has knotting simplicity. In the field of nucleic acid topology, “knotting” refers to nucleic acid that is intertwined many times and tied into knots (see, e.g., Buck D, Proceedings of Symposia in Applied Mathematics 2009; 66:1-33; Rybenkov V V et al. Proc Natl Acad Sci USA. 1993; 90 (11): 5307-5311). Knotting simplicity enables the RNA molecule(s) to avoid being kinetically trapped during the folding process, which can prevent proper folding into a user-defined target shape. Thus, in some embodiments, the crossing number of the nanostructure is zero and the nanostructure is unknotted. A crossing number is a knot invariant that shows the smallest number of crossings in any diagram of the knot, representing the topological complexity of a knot.

As used herein, the term “antibody” (Ab) includes, without limitation, a glycoprotein immunoglobulin which binds specifically to an antigen. In general, and antibody can comprise at least two heavy (H) chains and two light (L) chains interconnected by disulfide bonds, or an antigen-binding portion thereof. Each H chain comprises a heavy chain variable region (abbreviated herein as VH) and a heavy chain constant region. The heavy chain constant region comprises three constant domains, CH1, CH2 and CH3. Each light chain comprises a light chain variable region (abbreviated herein as VL) and a light chain constant region. The light chain constant region comprises one constant domain, CL. The VH and VL regions are further subdivided into regions of hypervariability, termed complementarity determining regions (CDRs), interspersed with regions that are more conserved, termed framework regions (FR). Each VH and VL comprises three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the Abs may mediate the binding of the immunoglobulin to host tissues or factors, including various cells of the immune system (e.g., effector cells) and the first component (C1q) of the classical complement system. An immunoglobulin may derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the Ab class or subclass (e.g., IgM or IgG1) that is encoded by the heavy chain constant region genes. The term “antibody” can include or exclude both naturally occurring and non-naturally occurring Abs; monoclonal and polyclonal Abs; chimeric and humanized Abs; human or nonhuman Abs; wholly synthetic Abs; and single chain Abs. A nonhuman Ab may be humanized by recombinant methods to reduce its immunogenicity in man. The term “antibody” also includes an antigen-binding fragment or an antigen-binding portion of any of the aforementioned immunoglobulins, and includes a monovalent and a divalent fragment or portion, and a single chain Ab.

“Antibody fragments” “nanobody fragments” or “sybody fragments” comprise only a portion of an intact antibody, nanobody or sybody, where the fragment retains at least one, and as many as most or all, of the functions normally associated with that fragment when present in an intact antibody, nanobody or sybody. In one embodiment, an antibody, nanobody or sybody fragment comprises an antigen binding site of the intact antibody, nanobody or sybody and thus retains the ability to bind antigen. In one embodiment, an antibody, nanobody, or sybody fragment is a monovalent antibody that has an in vivo half life substantially similar to an intact antibody.

As used herein, the term “affibody” refers to a small protein which specifically binds to a target with high affinity (Frejd et al., Experimental & Molecular Medicine volume 49, page e306 (2017)). Affibodies comprise alpha-helices and lack a bridging disulfide moiety. Affibodies of this disclosure can be purchased from Abcamm or made by protein expression methods well known in the art. In some embodiments, the affibody is ABY-025, which binds selectively to HER2 receptors with picomolar affinity.

Identity or homology with respect to a specified amino acid sequence of this invention is defined herein as the percentage of amino acid residues in a candidate sequence that are identical with the specified residues, after aligning the sequences and introducing gaps, if necessary, to achieve the maximum percent homology, and not considering any conservative substitutions as part of the sequence identity. None of N-terminal, C-terminal or internal extensions, deletions, or insertions into the specified sequence shall be construed as affecting homology. All sequence alignments called for in this invention are such maximal homology alignments. Generally, the nucleic acid sequence homology between the polynucleotides, oligonucleotides, and fragments of the invention and a nucleic acid sequence of interest will be at least 80%>, and more typically with preferably increasing homologies of at least 85%, 90%, 91%, 92%, 92%, 94%, 95%, 96%, 97%, 98%, 99%, and/or 100%. Two amino acid sequences are homologous if there is a partial or complete identity between their sequences.

In some embodiments, the practice of the present disclosure will employ, unless otherwise indicated molecular biology, microbiology, recombinant DNA, and immunology techniques within the skill of the art. Such techniques include techniques set forth in, for example, Molecular Cloning A Laboratory Manual, 2nd Ed., ed. by Sambrook, Fritsch and Maniatis (Cold Spring Harbor Laboratory Press, 1989); DNA Cloning, Volumes I and II (D. N. Glover ed., 1985); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Antibodies: A Laboratory Manual, by Harlow and Lane s (Cold Spring Harbor Laboratory Press, 1988); and Handbook Of Experimental Immunology, Volumes I-IV (D. M. Weir and C. C. Blackwell, eds., 1986).

The following terms are used to describe the sequence relationships between two or more nucleotide sequences: “reference sequence,” “comparison window,” “sequence identity,” “percentage of sequence identity,” “substantial identity,” and “complementarity”. As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally is 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison can be performed. Thus, the determination of percent identity, including sequence complementarity, between any two sequences is accomplished using a mathematical algorithm. Non-limiting examples of such mathematical algorithms are the algorithm of Myers and Miller (Myers and Miller, CABIOS, 4, 11 (1988)); the local homology algorithm of Smith et al. (Smith et al., Adv. Appl. Math., 2, 482 (1981)); the homology alignment algorithm of Needleman and Wunsch (Needleman and Wunsch, JMB, 48, 443 (1970)); the search-for-similarity-method of Pearson and Lipman (Pearson and Lipman, Proc. Natl. Acad. Sci. USA, 85, 2444 (1988)); the algorithm of Karlin and Altschul (Karlin and Altschul, Proc. Natl. Acad. Sci. USA, 87, 2264 (1990)), modified as in Karlin and Altschul (Karlin and Altschul, Proc. Natl. Acad. Sci. USA 90, 5873 (1993)).

Computer implementations of these mathematical algorithms is utilized for comparison of sequences to determine sequence identity or complementarity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs is performed using the default parameters. The CLUSTAL program is well described by Higgins et al. (Higgins et al., CABIOS, 5, 151 (1989)); Corpet et al. (Corpet et al., Nucl. Acids Res., 16, 10881 (1988)); Huang et al. (Huang et al., CABIOS, 8, 155 (1992)); and Pearson et al. (Pearson et al., Meth. Mol. Biol., 24, 307 (1994)). The ALIGN program is based on the algorithm of Myers and Miller, supra. The BLAST programs of Altschul et al. (Altschul et al., JMB, 215, 403 (1990)) are based on the algorithm of Karlin and Altschul supra.

Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score is increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P (N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, less than about 0.01, or even less than about 0.001.

To obtain gapped alignments for comparison purposes, Gapped BLAST (in BLAST 2.0) is utilized. Alternatively, PSI-BLAST (in BLAST 2.0) is used to perform an iterated search that detects distant relationships between molecules. When utilizing BLAST, Gapped BLAST, PSI-BLAST, the default parameters of the respective programs (e.g., BLASTN for nucleotide sequences, BLASTX for proteins) is used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. Alignment may also be performed manually by inspection.

For purposes of the present invention, comparison of nucleotide sequences for determination of percent sequence identity may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the program.

As used herein, the terms “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection.

As used herein, the term, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

As used herein, the term “identity” or “substantial identity” of polynucleotide sequences means that a polynucleotide comprises a sequence that has at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, or 94%, or even at least 95%, 96%, 97%, 98%, or 99% sequence identity, compared to a reference sequence using one of the alignment programs described using standard parameters.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Another indication that nucleotide sequences are substantially identical is if two molecules hybridize to each other under stringent conditions. Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that is accommodated by reducing the stringency of the hybridization media to achieve the desired detection of the target nucleic acid sequence.

The term “complementary” as used herein refers to the broad concept of complementary base pairing between two nucleic acids aligned in an antisense position in relation to each other. When a nucleotide position in both of the molecules is occupied by nucleotides normally capable of base pairing with each other, then the nucleic acids are considered to be complementary to each other at this position. Thus, two nucleic acids are substantially complementary to each other when at least about 50%, at least about 60%, or at least about 80% of corresponding positions in each of the molecules are occupied by nucleotides which normally base pair with each other (e.g., A: T (A: U for RNA) and G: C nucleotide pairs).

As used herein, the term “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

As used herein, the term “encoding” refers to the innate property of specific sequences of nucleotides in a polynucleotide, including a gene, a cDNA, or an mRNA, to serve as templates for the synthesis of other biopolymers having either a defined sequence of nucleotides or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and the non-coding strand, used as the template for transcription of a gene or DNA, can be referred to as encoding the protein or other product of that gene.

As used herein, the term “derived” or “directed to” with respect to a nucleotide molecule means that the molecule has complementary sequence identity to a particular molecule of interest.

As used herein, the term “aptamer” refers to a nucleic acid sequence that interacts with a ligand under normal physiological conditions.

As used herein, the term “lipid nanoparticle” or “LNP” refers to a cell, or model cell, comprising a lipid bilayer. The lipid bilayer can include or exclude a lipid selected from: DOTAP (1,2-dioleoyl-3-trimethylammonium-propane chloride salt), DOPC (1,2-dioleoyl-sn-glycero-3-phosphocholine), cholesteryl hemisuccinate (CHEMS), 1-stearoyl-2-hydroxy-sn-glycero-3-phosphate (sodium salt) (LPA), dioctanoylglycerol pyrophosphate (ammonium salt) (DGPP), 5-(palmitoyloxy) octadecanoic acid (5-PAHSA), 9-(palmitoyloxy) octadecanoic acid (9-PAHSA), 2-hydroxyoleic acid (sodium salt) (2-OHOA), mycolic acid, N-oleoylglycine, N-arachidonoylglycine, N-palmitoylglycine, Galactocerebroside, Gm1 ganglioside, and other cell membrane lipids including those referred to in Alberts et al., Molecular Biology of the Cell. 4th edition, New York: Garland Science; 2002, herein incorporated by reference. In some embodiments, the lipid nanoparticle is a lipid nanoparticle described in U.S. Pat. Nos. 10,799,463, 10485884, 10507249, 10166298, 11337922, 9580711, 9567296, U.S. Patent Application Publication Nos. US20220409536, US20220062175, US20200129445, US20200315967, US20230149310, US20210121411, US20180153822, and US20110038941.

As used herein, the term “therapeutically effective amount” means an amount of a compound of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

As used herein, the term “subject” as used herein refers to humans, higher non-human primates, rodents, domestic, cows, horses, pigs, sheep, dogs and cats. In one embodiment, the subject is a human.

The term “therapeutically effective amount,” in reference to treating a disease state/condition, refers to an amount of a therapeutic agent that is capable of having any detectable, positive effect on any symptom, aspect, or characteristics of a disease state/condition when administered as a single dose or in multiple doses. Such effect need not be absolute to be beneficial.

As used herein, the terms “treat” and “treatment” refer to both therapeutic treatment and prophylactic or preventative measures, wherein the object is to prevent or decrease an undesired physiological change or disorder. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder and those prone to have the condition or disorder or those in which the condition or disorder is to be prevented.

The terms “inhibiting” or “reducing” or any variation of these terms includes any measurable decrease or complete inhibition to achieve a desired result. The terms “promote” or “increase” or any variation of these terms includes any measurable increase or production of a protein or molecule to achieve a desired result.

The term “preventing” or any variation of this term means to slow, stop, or reverse progression, in whole or in part, or ameliorate toward a result. The prevention may be any slowing of the progression toward the result.

As used herein, the term “cancer” refers to a broad group of various diseases characterized by the uncontrolled growth of abnormal cells in the body. Unregulated cell division and growth results in the formation of malignant tumors that invade neighboring tissues and may also metastasize to distant parts of the body through the lymphatic system or bloodstream. In some embodiments, a “cancer” or “cancer tissue” can include a tumor. The types of cancers that are treated by the methods of this disclosure can include or exclude cancers of the immune system including lymphoma, acute lymphoblastoid leukemia, leukemia, and other leukocyte malignancies. In some embodiments, the methods of this disclosure reduce the tumor size of a tumor derived from, for example, bone cancer, pancreatic cancer, breast cancer, brain cancer, lung cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma (NHL), primary mediastinal large B cell lymphoma (PMBC), diffuse large B cell lymphoma (DLBCL), follicular lymphoma (FL), transformed follicular lymphoma, splenic marginal zone lymphoma (SMZL), cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukemia, acute myeloid leukemia, chronic myeloid leukemia, acute lymphoblastic leukemia (ALL) (including non T cell ALL), chronic lymphocytic leukemia (CLL), solid tumors of childhood, lymphocytic lymphoma, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, and combinations of said cancers. In some embodiments, the cancer is acute lymphoblastoid leukemia.

As used herein, the term “anti-cancer agent” or “cancer therapeutic agent” refers to an agent which has been identified to elicit a cancer killing response. In some embodiments, the anticancer agent is selected from: a chemotherapeutic agent. In certain embodiments, the chemotherapeutic agent is selected from: Abraxane (chemical name: albumin-bound or nab-paclitaxel), Adriamycin (chemical name: doxorubicin), carboplatin (brand name: Paraplatin), Cytoxan (chemical name: cyclophosphamide), daunorubicin (brand names: Cerubidine, DaunoXome), Doxil (chemical name: doxorubicin), Ellence (chemical name: epirubicin), fluorouracil (also called 5-fluorouracil or 5-FU; brand name: Adrucil), Gemzar (chemical name: gemcitabine), Halaven (chemical name: eribulin), Ixempra (chemical name: ixabepilone), methotrexate (brand names: Amethopterin, Mexate, Folex), Mitomycin (chemical name: mutamycin), mitoxantrone (brand name: Novantrone), Navelbine (chemical name: vinorelbine), Taxol (chemical name: paclitaxel), Taxotere (chemical name: docetaxel), thiotepa (brand name: Thioplex), vincristine (brand names: Oncovin, Vincasar PES, Vincrex), and Xeloda (chemical name: capecitabine). In certain embodiments, the chemotherapeutic agent is selected from: Abraxane (Paclitaxel (with albumin) Injection), Adriamycin (Doxorubicin), Afinitor (Everolimus), Alecensa (Alectinib), Alimta (PEMETREXED), Aliqopa (Copanlisib), Alkeran Injection (Melphalan), Alunbrig (Brigatinib), Aredia (Pamidronate), Arimidex (Anastrozole), Aromasin (Exemestane), Arranon (Nelarabine), Arzerra (Ofatumumab), Avastin (Bevacizumab), Bavencio (Avelumab), Beleodaq (Belinostat), Besponsa (Inotuzumab Ozogamicin), Bexxar (Tositumomab), BiCNU (Carmustine), Blenoxane (Bleomycin), Blincyto (Blinatumomab), Bosulif (Bosutinib), Braftovi (Encorafenib), Busulfex (Busulfan), Cabometyx (Cabozantinib), Calquence (Acalabrutinib), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Cinqair (Reslizumab), Clolar (Clofarabine), Cometriq (Cabozantinib), Copiktra (Duvelisib), Cosmegen (Dactinomycin), Cotellic (Cobimetinib), Cyramza (Ramucirumab), CytosarU (Cytarabine), Cytoxan (Cytoxan), Cyclophosphamide, Dacogen (Decitabine), Darzalex (Daratumumab), DaunoXome (Daunorubicin Lipid Complex), Daurismo (Glasdegib), Decadron (Dexamethasone), DepoCyt (Cytarabine Lipid Complex), Dexamethasone Intensol (Dexamethasone), Dexpak Taperpak (Dexamethasone), Docefrez (Docetaxel), Doxil (Doxorubicin Lipid Complex), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Emend (Fosaprepitant), Empliciti (Elotzumab), Erbitux (Cetuximab), Erivedge (Vismodegib), Erleada (Apalutamide), Erwinaze (Asparaginase Erwinia chrysanthemi), Ethyol (Amifostine), Etopophos (Etoposide), Eulexin (Flutamide), Fareston (Toremifene), Farydak (Panobinostat), Faslodex (Fulvestrant), Femara (Letrozole), Firmagon (Degarelix), FloPred (Prednisolone), Fludara (Fludarabine), Folex(Methotrexate), Folotyn (Pralatrexate), FUDR (FUDR (floxuridine)), Gazyva (Obinutuzumab), Gemzar (Gemcitabine), Gilotrif (Afatinib), Gleevec (Imatinib Mesylate), Halaven (Eribulin), Herceptin (Trastuzumab), Hexalen (Altretamine), Hycamtin (Topotecan), Hycamtin (Topotecan), Hydrea (Hydroxyurea), Ibrance (Palbociclib), Iclusig (Ponatinib), Idamycin PFS (Idarubicin), Idhifa (Enasidenib), Ifex (Ifosfamide), Imbruvica (Ibrutinib), Imfinzi (Durvalumab), Imlygic (Talimogene Laherparepvec), Inlyta (Axitinib), Intron A alfab (Interferon alfa-2a), Iressa (Gefitinib), Istodax (Romidepsin), Ixempra (Ixabepilone), Jakafi (Ruxolitinib), Jevtana (Cabazitaxel), Kadcyla (Ado-trastuzumab Emtansine), Keytruda (Pembrolizumab), Kisqali (Ribociclib), Kyprolis (Carfilzomib), Lanvima (Lenvatinib), Leukeran (Chlorambucil), Leukine (Sargramostim), Leustatin (Cladribine), Lorbrena (Lorlatinib), Lupron (Leuprolide), Lynparza (Olaparib), Lysodren (Mitotane), Matulane (Procarbazine), Megace (Megestrol), Mekinist (Trametinib), Mektovi (Binimetinib), Mesnex (Mesna), Mustargen (Mechlorethamine), Mutamycin (Mitomycin), Myleran (Busulfan), Mylotarg (Gemtuzumab Ozogamicin), Navelbine (Vinorelbine), Nerlynx (Neratinib), Neulasta (filgrastim), Neulasta (pegfilgrastim), Neupogen (filgrastim), Nexavar (Sorafenib), Nilandron (Nilandron (nilutamide)), Ninlaro (Ixazomib), Nipent (Pentostatin), Nolvadex (Tamoxifen), Odomzo (Sonidegib), Oncaspar (Pegaspargase), Oncovin (Vincristine), Opdivo (Nivolumab), Panretin (Alitretinoin), Paraplatin (Carboplatin), Perjeta (Pertuzumab), Platinol (Cisplatin), PlatinolAQ (Cisplatin), Pomalyst (Pomalidomide), Portrazza (Necitumumab), Proleukin (Aldesleukin), Purinethol (Mercaptopurine), Reclast (Zoledronic acid), Revlimid (Lenalidomide), Rituxan (Rituximab), RoferonA alfaa (Interferon alfa-2a), Rubex (Doxorubicin), Rubraca (Rucaparib), Rydapt (Midostaurin), Sandostatin (Octreotide), Soltamox (Tamoxifen), Sprycel (Dasatinib), Stivarga (Regorafenib), Sutent (Sunitinib), Sylvant (Siltuximab), Synribo (Omacetaxine), Tabloid (Thioguanine), Taflinar (Dabrafenib), Tagrisso (Osimertinib), Talzenna (Talazoparib), Tarceva (Erlotinib), Targretin Capsules (Bexarotene), Tasigna (Decarbazine), Taxol (Paclitaxel), Taxotere (Docetaxel), Tecentriq (Atezolizumab), Temodar (Temozolomide), Tepadina (Thiotepa), Thioplex (Thiotepa), Tibsovo (Ivosidenib), Toposar (Etoposide), Torisel (Temsirolimus), Treanda (Bendamustine hydrochloride), Trelstar (Triptorelin), Tykerb (lapatinib), Unituxin (Dinutuximab), Valstar (Valrubicin), Varubi (Rolapitant), Vectibix (Panitumumab), Velban (Vinblastine), Velcade (Bortezomib), Venclexta (Venetoclax), Vepesid (Etoposide), Vepesid (Etoposide Injection), Verzenio (Abemaciclib), Vesanoid (Tretinoin), Vidaza (Azacitidine), Vincasar PFS (Vincristine), Vincrex (Vincristine), Vistogard (Uridine Triacetate), Vitrakvil (Larotrectinib), Vizimpro (Dacomitinib), Votrient (Pazopanib), Vumon (Teniposide), Wellcovorin IV (Leucovorin), Xalkori (Crizotinib), Xeloda (Capecitabine), Xospata (Gilteritinib), Xtandi (Enzalutamide), Yervoy (Ipilimumab), Yescarta (Axicabtagene), Yondelis (Trabectedin), Zaltrap (Ziv-aflibercept), Zanosar (Streptozocin), Zejula (Niraparib), Zelboraf (Vemurafenib), Zevalin (Ibritumomab Tiuxetan), Zoladex (Goserelin), Zolinza (Vorinostat), Zometa (Zoledronic acid) Zortress (Everolimus),,Zydelig (Idelalisib), Zykadia (Ceritinib), and Zytiga (Abiraterone). In some embodiments, the anti-cancer agent is selected from: Alkylating Agents: Altretamine, Bendamustine, Busulfan, Carmustine, Chlorambucil, Cyclophosphamide, Dacarbazine, Ifosfamide, Lomustine, Mechlorethamine, Melphalan, Procarbazine, Streptozocin, Temozolomide, Thiotepa, Trabectedin; Platinum Coordination Complexes: Carboplatin, Cisplatin, Oxaliplatin; Bleomycin, Dactinomycin, Daunorubicin, Doxorubicin, Epirubicin, Idarubicin, Mitomycin, Mitoxantrone, Plicamycin, Valrubicin, Antimetabolites: Antifolates: Methotrexate, Pemetrexed, Pralatrexate, Trimetrexate; Purine Analogues: Azathioprine, Cladribine, Fludarabine, Mercaptopurine, Thioguanine; Pyrimidine Analogues: Azacitidine, Capecitabine, Cytarabine, Decitabine, Floxuridine, Fluorouracil, Gemcitabine, Trifluridine/Tipracil; Biologic Response Modifiers: Aldesleukin (IL-2), Denileukin Diftitox, Interferon Gamma; Histone Deacetylase Inhibitors: Belinostat, Panobinostat, Romidepsin, Vorinostat; Hormonal Agents: Antiandrogens: Abiraterone, Apalutamide, Bicalutamide, Cyproterone, Enzalutamide, Flutamide, Nilutamide; Antiestrogens (including Aromatase Inhibitors): Anastrozole, Exemestane, Fulvestrant, Letrozole, Raloxifene, Tamoxifen, Toremifene; Gonadotropin Releasing Hormone Analogues: Degarelix, Goserelin, Histrelin, Leuprolide, Triptorelin; Peptide Hormones: Lanreotide, Octreotide, Pasireotide; Monoclonal Antibodies: Alemtuzumab, Atezolizumab, Avelumab, Bevacizumab, Blinatumomab, Brentuximab, Cemiplimab, Cetuximab, Daratumumab, Dinutuximab, Durvalumab, Elotuzumab, Gemtuzumab, Inotuzumab Ozogamicin, Ipilimumab, Mogamulizumab, Moxetumomab Pasudotox, Necitumumab, Nivolumab, Ofatumumab, Olaratumab, Panitumumab, Pembrolizumab, Pertuzumab, Ramucirumab, Rituximab, Tositumomab, Trastuzumab, Protein Kinase Inhibitors, Abemaciclib, Acalabrutinib, Afatinib, Alectinib, Axitinib, Binimetinib, Bortezomib, Bosutinib, Brigatinib, Cabozantinib, Carfilzomib, Ceritinib, Cobimetinib, Copanlisib, Crizotinib, Dabrafenib, Dacomitinib, Dasatinib, Duvelisib, Enasidenib, Encorafenib, Erlotinib, Gefitinib, Gilteritinib, Glasdegib, Ibrutinib, Idelalisib, Imatinib, Ivosidenib, Ixazomib, Lapatinib, Larotrectinib, Lenvatinib, Lorlatinib, Midostaurin, Neratinib, Nilotinib, Niraparib, Olaparib, Osimertinib, Palbociclib, Pazopanib, Pexidartinib, Ponatinib, Regorafenib, Ribocicib, Rucaparib, Ruxolitinib, Selumetinib, Sonidegib, Sorafenib, Sunitinib, Talazoparib, Trametinib, Vandetanib, Vemurafenib, Vismodegib, Zanubrutinib; Taxanes: Cabazitaxel, Docetaxel, Paclitaxel; Topoisomerase Inhibitors: Etoposide, Irinotecan, Teniposide, Topotecan; Vinca Alkaloids: Vinblastine, Vincristine, Vinorelbine; or Asparaginase (Pegaspargase), Bexarotene, Eribulin, Everolimus, Hydroxyurea, Ixabepilone, Lenalidomide, Mitotane, Omacetaxine, Pomalidomide, Tagraxofusp, Telotristat, Temsirolimus, Thalidomide, or Venetoclax.

As used herein, the term “anti-tumor effect” refers to a biological effect that can present as a decrease in tumor volume, a decrease in the number of tumor cells, a decrease in tumor cell proliferation, a decrease in the number of metastases, an increase in overall or progression-free survival, an increase in life expectancy, or amelioration of various physiological symptoms associated with the tumor.

As used herein, the term “progression-free survival,” or “PFS,” refers to the time from the treatment date to the date of disease progression per the revised IWG Response Criteria for Malignant Lymphoma or death from any cause.

As used herein, the terms “serum level” and “serum concentration” refer to the amount of an analyte in the serum of a subject. Serum levels of a given analyte are measured using any method known in the art. For example, cytokine serum levels are measured using an enzyme-linked immunosorbent assay (ELISA). In one particular embodiment, cytokine serum levels are measured using an EMDmillipore LUMINEX® xMAP® multiplex assay.

As used herein, the term “dosing interval,” refers to the amount of time that elapses between multiple doses of a formulation disclosed herein being administered to a subject. Dosing interval can thus be indicated as ranges.

Doses can be presented as a “weight based dose” or as a “body surface area (BSA) based dose.” A weight based dose is a dose that is administered to a patient that is calculated based on the weight of the patient, e.g., mg/kg. A BSA based dose is a dose that is administered to a patient that is calculated based on the surface area of the patient, e.g., mg/m2. The two forms of dose measurement can be converted for human dosing by multiplying the weight based dose by 37 or dividing the BSA based dose by 37. For example, a dose of 60 mg/kg to be administered to a human subject is equivalent to a 2220 mg/m2 dose of the same drug to be administered to the same subject.

As used herein, the term “dosing frequency” refers to the frequency of administering doses of a formulation disclosed herein in a given time. Dosing frequency can be indicated as the number of doses per a given time.

As described herein, any concentration range, percentage range, ratio range or integer range is to be understood to include the value of any integer within the recited range and, when appropriate, fractions thereof (including one-tenth and one-hundredth of an integer), unless otherwise indicated.

The tumor microenvironment is an important aspect of cancer biology that contributes to tumor initiation, tumor progression and responses to therapy. The tumor microenvironment is composed of a heterogeneous cell population that includes malignant cells and cells that support tumor proliferation, invasion, and metastatic potential though extensive crosstalk. Tumor cells often induce an immunosuppressive microenvironment, which favors the development of immunosuppressive populations of immune cells, such as myeloid-derived suppressor cells (MDSCs), tumor-associated macrophage (TAM), and regulatory T cells (Tregs).

Therefore, targets within the tumor microenvironment have been uncovered that can help direct and improve the actions of various cancer therapies, notably immunotherapies that work by potentiating host antitumor immune responses.

### Certain Modular RNA Carrier Systems

In some embodiments, this disclosure provides for modular RNA carrier systems, which can be complexes, nucleic acid nanodevices, or RNA carriers. In a modular RNA carrier system, RNA acts as a structural element and/or an informational element. Modular RNA carrier systems can also serve as a scaffold for the formation of other nucleic acid-based structures (e.g., nucleic acid nanodevices). Nucleic acid nanodevices may be prepared by methods using one or more nucleic acid oligonucleotides. For example, such nanostructures may be assembled based on the concept of base-pairing. While no specific sequence is required, the sequences of each oligonucleotide must be partially complementary to certain other oligonucleotides to enable hybridization of all strands or sequences within a single oligonucleotide to enable hybridization and assembly of the nanostructure. For example, in certain embodiments, the nucleic acid nanodevice is an DNA rectangle nanostructure, self-assembled from one single-stranded DNA molecule.

### Structural Aspects of Modular RNA Carrier Systems

The modular RNA carrier systems include structural features which serve to enhance the efficacy of the delivered RNA in part from the RNA or DNA carrier. In some embodiments, the modular RNA carrier system is designed to have all or a portion of the oligonucleotide sequence to be complementary to all or a portion of a ssDNA or ssRNA oligonucleotide. In some embodiments, the oligonucleotide sequence can comprise RNA, DNA, modified RNA, modified DNA, or combinations thereof. In some embodiments, the 5′- or 3′-ends of the oligonucleotide is modified with a oligonucleoside which can bind to a polypeptide.

In some embodiments, the oligonucleotide is 5′-modified with a thiol-comprising nucleotide. In some embodiments, the thiol-comprising nucleotide is further reacted with a cross-functional linker. In some embodiments, the cross-functional linker is sulfo-SMCC ((sulfosuccinimidyl 4-(N-maleimidomethyl)cyclohexane-1-carboxylate), ThermoFisher cat. 22322). In some embodiments, the cross-functional linker is Sulfo-LC-SPDP (sulfosuccinimidyl 6-(3′-(2-pyridyldithio) propionamido) hexanoate) (ThermoFisher cat. 21650). In some embodiments, the sequence of the oligonucleotide sequence which is complementary to all or a portion of a ssDNA is selected from the following loading oligonucleotide sequences (Loading Oligonucleotides): SEQ ID Nos. 1-12.

Any of the aforementioned oligonucleotide sequences is 5′ or 3′ modified as described herein.

In some embodiments, the cross-functional compound can further be reacted with an agent. In some aspects, the agent is a protein or peptide comprising a lysine amino acid. The amine on the lysine can react with the amino-reactive cross-functional compound to form a loading oligonucleotide-functionalized agent. In some embodiments, the oligonucleotide-functionalized agent is hybridized to a portion or all of the ssDNA.

In certain embodiments, the modular RNA carrier system can include or exclude the use of “staple strands.” It was surprisingly found that in some embodiments, the modular RNA carrier system can self-assemble with no added stable strands. As used herein, the term “staple strands” refers to short single-stranded oligonucleotides of about 20 to about 40 nucleotides in length, such as 20, 21, 22, 23, 24, 25, 26,27, 28, 29, 30, 31, 32, 33, 34, 35, 36, 37, 38, 39, or 40 nucleotides in length, wherein one end of the staple strand hybridizes with a region of the scaffold strand, and the second end of the staple strand hybridizes with another region of the scaffold strand, thereby “stapling” the two regions of the scaffold strand. Exemplary staple strand sequences can those disclosed in US20200390814, which is herein incorporated by reference.

As used herein, the term “scaffold strand” refers to a polynucleotide sequence which is about 45 to about 15,000 nucleotides in length, and can form tertiary structures via Watson-Crick base pairing and/or intra-duplex interactions.

The assembly of such modular RNA carrier systems may be based on base-pairing principles or other non-canonical binding interactions. For example, while no specific DNA sequence is required, regions of complementary within a single DNA molecule or between multiple DNA molecules may be used for assembly. Persons of ordinary skill in the art will readily understand and appreciate that the optimal sequence for any given modular RNA carrier system will depend on the selected shape, size, nucleic acid content, and selected use of such NA structure. In certain embodiments, wherein the nanostructure comprises more than one ssRNA molecule (e.g. two or more oligonucleotides/polynucleotides), each ssRNA molecule may have a region that is complementary to a region on another ssRNA molecule to enable hybridization of the strands and assembly of the nanostructure. In certain other embodiments, wherein the nanostructure consists of a single ssRNA molecule (i.e., a single unimolecular RNA oligonucleotide/polynucleotide), regions within the molecule may be complementary to certain other regions within the molecule to enable hybridization and assembly of the nanostructure. Modular RNA carrier systems produced in accordance with the present disclosure are typically nanometer-scale structures (e.g., having length scale of 1 to 1000 nanometers), although, in some instances, the term “nanostructure” herein may refer to micrometer-scale structures (e.g., assembled from more than one nanometer-scale or micrometer-scale structure). In some embodiments, a Nucleic acid nanodevice described herein has a length scale of 1 to 1000 nm, 1 to 900 nm, 1 to 800 nm, 1 to 700 nm, 1 to 600 nm, 1 to 500 nm, 1 to 400 nm, 1 to 300 nm, 1 to 200 nm, 1 to 100 nm or 1 to 50 nm. In some embodiments, a modular RNA carrier system described herein has a length scale of greater than 1000 nm. In some embodiments, a modular RNA carrier system described herein has a length scale of 1 micrometer to 2 micrometers.

In certain embodiments, the modular RNA carrier system comprises, consists essentially of, or consists of multiple ssRNA molecules (e.g., more than one oligonucleotide/polynucleotide strands, such as two or more ssRNA molecules). In certain embodiments, the RNA oligonucleotide molecule carrier comprises two or more ssRNA molecules, which are capable of self-assembling (or configured to self-assemble) into a nanostructure. In certain embodiments, the modular RNA carrier system is assembled from two or more ssRNA molecules through paranemic cohesion crossovers. Thus, in certain embodiments, the modular RNA carrier system comprises two or more ssRNA molecules, wherein the ssRNA molecules self-assemble to form at least one paranemic cohesion crossover.

In some embodiments, the modular RNA carrier system comprises programmable RNA. Programmable mRNA (also referred to herein as “Pro-mRNA” or “pmrna”) can translate the apoptotic protein to trigger cancer cell death. In some embodiments, Pro-mRNA is activated upon a selected signal event. In some embodiments, the selected signal event is the presence of, or a concentration of, a selected biomolecule. In some embodiments, the biomolecule is a protein, peptide, amino acid, hormone, steroid, or nucleic acid sequence.

In certain embodiments, the modular RNA carrier system comprises, consists essentially of, or consists of a single ssRNA molecule (i.e., one unimolecular oligonucleotide/polynucleotide strand). In certain embodiments, the modular RNA carrier system is assembled using one ssRNA molecule (e.g., in certain embodiments one and only one, exactly one, or greater than zero and less than two). In certain embodiments, the modular RNA carrier system is comprised of one ssRNA molecule, which is capable of self-assembling into a nanostructure. In certain embodiments, the modular RNA carrier system consists of one ssRNA molecule, which is capable of self-assembling into a nanostructure.

The length of each RNA strand is variable and depends on, for example, the type of nanostructure to be formed. In certain embodiments, the modular RNA carrier system is comprised of multiple oligonucleotide strands. In certain embodiments, the oligonucleotide strands can be made of RNA, DNA, or both. In certain embodiments, the modular RNA carrier system is comprised of a single (i.e., unimolecular) oligonucleotide strand. In certain embodiments, the oligonucleotide or RNA strand is about 15 nucleotides in length to about 150,000 nucleotides in length, about 15 to about 7500 nucleotides in length, about 3000 to about 7000 nucleotides in length, about 5000 to about 7000 nucleotides in length, about 5500 to about 6500 nucleotides in length, about 15 to about 5000 nucleotides in length, about 15 to about 4000 nucleotides in length, about 15 to about 3000 nucleotides in length, about 250 to about 3000 nucleotides in length, about 500 to about 3000 nucleotides in length, about 1000 to about 3000 nucleotides in length, about 1500 to about 2500 nucleotides in length, or between any of the aforementioned nucleotide lengths. In certain embodiments, the at least one ssRNA molecule (i.e., oligonucleotide or RNA strand) is about 10 nucleotides in length to about 200,000 nucleotides in length, the at least one ssRNA molecule (i.e., oligonucleotide or RNA strand) is about 10 nucleotides in length to about 100,000 nucleotides in length, the at least one ssDNA molecule (i.e., oligonucleotide or RNA strand) is about 10 nucleotides in length to about 90,000 nucleotides in length, about 10 to about 80,000 nucleotides in length, about 10 to about 70,000 nucleotides in length, about 10 to about 60,000 nucleotides in length, about 10 to about 50,000 nucleotides in length, about 10 to about 40,000 nucleotides in length, about 10 to about 30,000 nucleotides in length, about 10 to about 25,000 nucleotides in length, or about 10 to about 20,000 nucleotides in length. In certain embodiments, the ssRNA molecule (i.e., oligonucleotide or RNA strand) is about 15 nucleotides in length to about 20,000 nucleotides in length, the ssRNA molecule (i.e., oligonucleotide or RNA strand) is about 15 nucleotides in length to about 10,000 nucleotides in length, about 15 to about 7500 nucleotides in length, about 3000 to about 7000 nucleotides in length, about 5000 to about 7000 nucleotides in length, about 1500 to about 6500 nucleotides in length, about 1000 to about 7000 nucleotides in length, about 5500 to about 6500 nucleotides in length, about 15 to about 5000 nucleotides in length, about 15 to about 4000 nucleotides in length, about 15 to about 3000 nucleotides in length, about 250 to about 3000 nucleotides in length, about 500 to about 3000 nucleotides in length, about 1000 to about 3000 nucleotides in length, or about 1500 to about 2500 nucleotides in length. In certain embodiments, the ssRNA molecule (i.e., oligonucleotide or RNA strand) is about 100, about 200, about 300, about 400, about 500, about 600, about 700, about 800, about 900, about 1000, about 1100, about 1200, about 1300, about 1400, about 1500, about 1600, about 1700, about 1800, about 1900, about 2000, about 2100, about 2200, about 2300, about 2400, about 2500, about 2600, about 2700, about 2800, about 2900, about 3000, about 3100, about 3200, about 3300, about 3400, about 3500, about 3600, about 3700, about 3800, about 3900, about 4000, about 4100, about 4200, about 4300, about 4400, about 4500, about 4600, about 4700, about 4800, about 4900, about 5000, about 5100, about 5 5200, about 5300, about 5400, about 5500, about 5600, about 5700, about 5800, about 5900, about 6000, about 6100, about 6200, about 6300, about 6400, about 6500, about 6600, about 6700, about 6800, about 6900, about 7000, about 7100, about 7200, about 7300, about 7400, about 7500, about 7600, about 7700, about 7800, about 7900, about 8000, about 8100, about 8200, about 8300, about 8400, about 8500, about 8600, about 8700, about 8800, about 8900, about 9000, about 9100, about 9200, about 9300, about 9400, about 9500, about 9600, about 9700, about 9800, about 9900, about 10000, about 10100, about 10200, about 10300, about 10400, about 10500, about 10600, about 10700, about 10800, about 10900, about 11000, about 11000, about 11100, about 11200, about 11300, about 11400, about 11500, about 11600, about 11700, about 11800, about 11900, about 12000, about 12100, about 12200, about 12300, about 12400, about 12500, about 12600, about 12700, about 12800, about 12900 nucleotides in length, about 13000 nucleotides in length, about 14000 nucleotides in length, about 15000 nucleotides in length, about 16000 nucleotides in length, about 17000 nucleotides in length, about 18000 nucleotides in length, about 19000 nucleotides in length, about 20000 nucleotides in length, about 25000 nucleotides in length, about 30000 nucleotides in length, about 35000 nucleotides in length, about 40000 nucleotides in length, about 45000 nucleotides in length, about 50000 nucleotides in length, about 75000 nucleotides in length, about 100000 nucleotides in length, about 125000 nucleotides in length, about 150000 nucleotides in length, about 175000 nucleotides in length or about 200000 nucleotides in length.

In some embodiments, the DNA or RNA is synthesized de novo using chemical or biological methods. The DNA or RNA can be chemically synthesized in a step-wise manner. In some embodiments, the DNA or RNA can be synthesized using the cyanoethyl phosphoramidite method (Beaucage, S. L., and Caruthers, M. H., Tet. Let. 22:1859,1981);

nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622,1988). These chemistries can be performed by a variety of automated oligonucleotide synthesizers available in the market.

In certain embodiments, about 60-99% of the modular RNA carrier system is double stranded and about 1-40% of the modular RNA carrier system is single stranded.

In certain embodiments, about 95% of the modular RNA carrier system is double stranded and about 5% of the modular RNA carrier system is single stranded.

In certain embodiments, the modular RNA carrier system comprises a rectangular sheet nanostructure.

In certain embodiments, the modular RNA carrier system comprises a nucleic acid sequence about 1500 to about 2500 nucleotides in length.

In certain embodiments, the nucleic acid sequence of the RNA carrier system is about 1500 to about 2500 nucleotides in length.

In certain embodiments, the RNA sequences described herein comprise one or more modified nucleic acids. The modified nucleic acids can be a modified ribonucleotide or ribonucleoside. In some embodiments, the modified ribonucleoside can be selected from the NA-incorporated from, in part. 5-Aminoallyluridine-5′-Triphosphate (Trilink, N-1062), biotin-16-Aminoallyluridine-5′-Triphosphate (Trilink, N-5005), or 5-DBCO-PEG4-UTP.

In some embodiments, this disclosure includes a targeting agent that comprises a nucleic acid nanodevice which can optionally comprise a targeting molecule. The nucleic acid nanodevice relies on nucleic acid self-assembly. There are several reasons for the success of DNA or RNA in nano-construction. First, Watson-Crick base-pairing between complementary DNA or RNA strands is highly predictable and stable. Second, the geometric features of the DNA or RNA double-helix is appropriate for nanoscale assembly: the helix is roughly 2 nm in diameter and 3.4 nm pitch per helical repeat. In addition, the development of several user-friendly software interfaces has facilitated the modeling of even the most intricate DNA or RNA nanostructures for experimental testing. Third, modern organic chemistry and molecular biology provide a diverse toolbox to readily synthesize, modify, and replicate DNA or RNA molecules at a relatively low cost. The biocompatibility of DNA or RNA makes it suitable for the construction of multicomponent nanostructures made from hetero-biomaterials with designed functions.

In some embodiments, the nucleic acid nanodevice designed based on the principle of DNA tile assembly (Winfree E, Liu F R, Wenzler L A, Seeman N C. Design and self-assembly of two-dimensional DNA crystals. Nature. 1998;394 (6693): 539-44. doi: Doi 10.1038/28998. PubMed PMID: ISI: 000075238700036; He Y, Chen Y, Liu H P, Ribbe A E, Mao CD. Self-assembly of hexagonal DNA two-dimensional (2D) arrays. Journal of the American Chemical Society. 2005;127 (35): 12202-3. doi: Doi 10.1021/Ja0541938. PubMed PMID: ISI: 000231637100027; Yan H, Park S H, Finkelstein G, Reif J H, LaBean T H. DNA-templated self-assembly of protein arrays and highly conductive nanowires. Science. 2003; 301 (5641): 1882-4. doi: DOI 10.1126/science. 1089389. PubMed PMID: ISI: 000185536700043; He Y, Tian Y, Ribbe A E, Mao C D. Highly connected two-dimensional crystals of DNA six-point-stars. Journal of the American Chemical Society. 2006; 128 (50): 15978-9. doi: Doi 10.1021/Ja0665141. PubMed PMID: ISI: 000242825600025, each of which is incorporated by reference). DNA tile assembly is a versatile and powerful method wherein small building blocks of DNA tiles, motifs, or even single-stranded DNA-along with the interactions between the building blocks—can be pre-designed using sticky-end cohesion. The recognition between sticky ends are programed with several algorithms that guide DNA building blocks to self-assemble into large 2D arrays with diverse topological and geometric features. In some embodiments, the DNA nanostructure is based on the principle of scaffolded DNA-origami (Rothemund, et al. Folding DNA to create nanoscale shapes and patterns. Nature. 2006;440 (7082): 297-302. doi: Doi 10.1038/Nature04586. PubMed PMID: WOS: 000235997600044; Douglas S M, Dietz H, Liedl T, Hogberg B, Graf F, Shih WM. Self-assembly of DNA into nanoscale three-dimensional shapes. Nature. 2009; 459 (7245): 414-8. doi: Doi 10.1038/Nature08016. PubMed PMID: ISI: 000266243700043; Han D R, Pal S, Nangreave J, Deng Z T, Liu Y, Yan H. DNA Origami with Complex Curvatures in Three-Dimensional Space. Science. 2011; 332 (6027): 342-6. doi: DOI 10.1126/science.1202998. PubMed PMID: ISI: 000289516600042; Han DR, Pal S, Yang Y, Jiang S X, Nangreave J, Liu Y, Yan H. DNA Gridiron Nanostructures Based on Four-Arm Junctions. Science. 2013; 339 (6126): 1412-5. doi: DOI 10.1126/science.1232252. PubMed PMID: ISI: 000316740700037, each of which is incorporated by reference). Scaffolded DNA origami is a technique which enables the folding of a long, single-stranded M13mp18 genomic DNA into a designed target shape by using a plurality of short DNA strand sequences referred to herein as “staple strands.” In some embodiments, the folding process is programmed resulting in the formation of structures in near-quantitative yields for many designs with unpurified staple strands. The inventors have surprisingly discovered that nucleic acid (DNA or RNA) nanostructures, including DNA origami, are fully addressable due to the unique sequence of long DNA or RNA, where each staple strand results in possible modification positions that can be linked to other functional materials with useful biological properties, for both DNA origami and other nucleic acid (DNA or RNA) nanostructures. In some embodiments, the staple strands comprise a polynucleotide with a portion of which is complementary to a second oligonucleotide, wherein the second oligonucleotide is connected to an other moiety. In some embodiments, the other moiety is selected from a protein tag, a targeting molecule, or combinations thereof. In some embodiments, the long genomic strand is RNA. In some embodiments, the long genomic strand is DNA.

In some embodiments, the RNA nanostructure (e.g., Pro-mRNA) has a shape selected from a semi-flat or flat sheet which may be in the form of a square or rectangle, a tube, a sphere, a tetrahedron, a box, a rectangle, and a round flat disc. The complex can be designed using algorithms to generate appropriate sequences for customized DNA or RNA structures to minimize unwanted interactions between DNA or RNA strands and help achieve a high folding yield of designed structures. Design parameters including the size, shape, and helical directions of Nucleic acid nanodevices can be optimized to improve targeting molecule loading efficiency of Nucleic acid nanodevices. The assembly of such DNA or RNA structures is achieved through thermal annealing processes and the obtained structures are purified with dialysis spin columns, gel electrophoresis methods, or size-exclusion chromatography methods. Atomic force microscopy (AFM) confirms the correct formation of the designed DNA nanostructure. In some embodiments, the DNA or RNA nanodevice can include or exclude those in U.S. Pat. Nos. 8,440,811; 8,552,167; 11,254,941; U.S. Patent Publication No. US20200390814; U.S. Patent Publication No. US20190240248; and U.S. Patent Publication No. US20220177887, all of which are herein incorporated by reference.

In some embodiments, the modular RNA carrier systems comprise a plurality of unique staple strands where each unique staple strand can serve as an addressable surface location to organize modular functional targeting molecules. Different targeting molecules are attached to the DNA or RNA nanostructure with a specific DNA or RNA sequence (“second oligonucleotide”) that is comprises a portion which is complementary to a portion of the sequence of staple strand sequence protruding from a DNA or RNA nanostructure (“first oligonucleotide”), whereby all the geometric parameters are precisely designed and have an addressable surface. In some embodiments, the staple strand sequences are oriented based on the conformations of the DNA or RNA nanostructure. The resulting construct enables the configuration of targeting molecules, including the distance between said molecules, their multivalency, and their relative geometry. Rows of different staple strands can be created to be at a precise distance apart to achieve optimal binding distance to increase the avidity of the targeting molecules to their cognate antigen.

In some embodiments, the modular RNA carrier systems are comprised of phosphorothioate backbones or locked nucleic acids to prevent nucleic acid degradation by nucleases. In other embodiments, the nucleic acid nanostructures can comprise an electrostatic coating (e.g. oligolysine-PEG conjugate), to simultaneously stabilize DNA or RNA nanostructures to low-magnesium conditions (including those encountered in the bloodstream) and help reduce degradation by endonucleases.

### Informational Aspects of Modular RNA Carrier Systems

In some embodiments, the modular RNA carrier systems comprise mRNA which act as informational element for the translation into a polypeptide or protein. In some embodiments, the modular RNA carrier systems further comprise methods of enhancing the mRNA translation by use of an enhancer.

Without being bound by theory, mRNA translational initiation is highly regulated. Initiation of protein synthesis from mRNA involves the orchestrated recruitment of the ribosome to the initiation codon by a complex of eukaryotic initiation factors (eIFs) that are present in limiting amounts to ensure controlled levels of protein synthesis (Jackson R J, et al., The mechanism of eukaryotic translation initiation and principles of its regulation. Nat Rev Mol Cell Biol. (2010); 11 (2): 113-27; Andaya A, et al., Phosphorylation stoichiometries of human eukaryotic initiation factors. Int J Mol Sci. (2014); 15 (7): 11523-38). Canonical translation initiation involves the proteins eIF4F which recognizes the mRNA 5′ G cap, a trimeric complex with cap binding (eIF4E), mRNA unwinding (eIF4A), and ribosomal loading (eIF4G) activities. The mRNA-bound eIF4F complex is then loaded into the 43S preinitiation complex comprised of the small ribosomal subunit (40S) and other auxiliary factors (eIF1, eIF2-tRNA, eIF3, and eIF5).

Translation initiation is regulated by secondary and tertiary structures within mRNAs that modulate eIF activity and can increase or decrease translation rates, such as encumbering eIF4A helicase unwinding by stable secondary structures or recruitment of activating auxiliary proteins (Truitt M L, et al., New frontiers in translational control of the cancer genome. Nat Rev Cancer. 2016; 16 (5): 288-304.). Internal ribosome entry sites (IRES) are RNA structures that facilitate direct entry of the translation machinery, which in some instances can bypass 5′ G cap recognition (Terenin I M, et al., A researcher's guide to the galaxy of IRESs. Cell Mol Life Sci. 2017; 74 (8): 1431-1455). In IRES-mediated translation initiation, eIF3 can recognizes the IRES structure directly (Querol-audi J, et al. Architecture of human translation initiation factor 3. Structure. 2013; 21 (6): 920-8; Valášek L S, et al. Embraced by eIF3: structural and functional insights into the roles of eIF3 across the translation cycle. Nucleic Acids Res. 2017; 45 (19): 10948-10968).

Specialized translation initiation is a novel form of regulation of protein synthesis whereby RNA structures within the 5′-UTR regulate translation rates of specific mRNAs. Like internal ribosomal entry sites (IRES), specialized translational initiation requires the recruitment of eukaryotic initiation factor 3 (eIF3), but specialized translation initiation requires cap recognition by eIF3d, a new 5′-m7GTP recognizing protein.

In some embodiments, the modular RNA carrier systems do not include an IRES. mRNAs lacking IRESs can still be translated when eIF4F is inhibited through an alternative form of translation initiation, that involves both recognition of specific mRNA structures by eIF3 and mRNA-cap recognition by eIF3d (Lee A S, et al., eIF3 targets cell-proliferation messenger RNAs for translational activation or repression. Nature. 2015; 522 (7554): 111-114; Lee A S, et al., eIF3d is an mRNA cap-binding protein that is required for specialized translation initiation. Nature. 2016; 536 (7614): 96-9).

In some embodiments, the modular RNA carrier systems include an IRES site. Inclusion of an IRES permits the translation of one or more open reading frames from a mRNA. The IRES element attracts a eukaryotic ribosomal translation initiation complex and promotes translation initiation. There are two known mechanisms by which translation is initiated in eukaryotes. The first is the canonical cap-dependent mechanism that is used by the most eukaryotic mRNAs, which requires an 5′ G cap, initiator Met-tRNAmet, initiation factor proteins, directional scanning, and GTP hydrolysis to place a translationally competent ribosome at the start codon. The second mechanism is cap-independent initiation. This mechanism bypasses the need for the cap and often many of the protein factors, using cis-acting IRES RNA elements to recruit the ribosome and initiate protein synthesis.

A number of linear IRES sequences are known and may be included in a recombinant nucleic acid molecule as described herein. For example, linear IRES sequences may be derived from a wide variety of viruses, such as from leader sequences of picoronaviruses (e.g., encephalomyocarditis virus (EMCV) UTR) (Jang et al., J. Virol., 63:1651-1660 (1989)), the polio leader sequence, the hepatitis A virus leader, the hepatitis C virus IRES, human rhinovirus type 2 IRES (Dobrikova et al., Proc. Natl. Acad. Sci., 100 (25): 15125-15130 (2003)), an IRES element from the foot and mouth disease virus (Ramesh et al., Nucl. Acid Res., 24:2697-2700 (1996)), and a giardiavirus IRES (Garlapati et al., J. Biol. Chem., 279 (5): 3389-3397 (2004)). A variety of nonviral IRES sequences also can be included in a mRNA sequence, including IRES sequences from yeast, the human angiotensin II type 1 receptor IRES (Martin et al., Mol. Cell Endocrinol., 212:51-61 (2003)), fibroblast growth factor IRESs (e.g., FGF-1 IRES and FGF-2 IRES, Martineau et al., Mol. Cell. Biol., 24 (17): 7622-7635 (2004)), vascular endothelial growth factor IRES (Baranick et al., Proc. Natl. Acad. Sci. U.S.A., 105 (12): 4733-4738 (2008); Stein et al., Mol. Cell. Biol., 18 (6): 3112-3119 (1998); Bert et al., RNA, 12 (6): 1074-1083 (2006)), and insulin-like growth factor 2 IRES (Pedersen et al., Biochem. J., 363 (Pt 1): 37-44 (2002)), the contents of each of which are herein incorporated by reference.

IRES sequences and vectors encoding IRES elements can be obtained from a variety of sources, such as, for example, Clontech (Mountain View, Calif.), Invivogen (San Diego, Calif.), Addgene (Cambridge, Mass.) and GeneCopoeia (Rockville, Md.), and IRESite: The database of experimentally verified IRES structures (iresite.org), or reprod.njmu.edu.cn/cgi-bin/iresbase/download.php.

In some embodiments, IRES sequences are those described in: Shira Weingarten-Gabbay et al., Systematic discovery of cap-independent translation sequences in human and viral genomes. Science 351, aad4939 (2016); DOI: 10.1126/science.aad4939; or Zhao, et al., IRESbase: A Comprehensive Database of Experimentally Validated Internal Ribosome Entry Sites, Genomics, Proteomics & Bioinformatics, (2020); 18 (2): 129-139; the contents of each of which are herein incorporated by reference.

As used herein, the terms “internal ribosome entry site,” “internal ribosome entry sequence,” “IRES” and “IRES sequence region” are used interchangeably herein and refer to cis elements of viral or human cellular RNAs (mRNA) that bypass the steps of canonical eukaryotic cap-dependent translation initiation. The canonical cap-dependent mechanism used by most eukaryotic mRNAs requires an 5′ G cap, an initiator Met-tRNAmet, initiation factor proteins, directional scanning, and GTP hydrolysis to place a translationally competent ribosome at the start codon. IRESs are comprised of a long and highly structured 5′-UTR which mediates the translation initiation complex binding and catalyzes the formation of a functional ribosome.

Exemplary sequences encoding IRESs for use in the RNA molecules of the present disclosure are set forth in the sequences of SEQ ID NO: 1-228 or SEQ ID NO: 229-17201 in U.S. Pat. No. 11,685,924, herein incorporated by reference. In some embodiments, the sequences encoding IRES have 70%-99% homology to SEQ ID NOs. 1-228 or SEQ ID NO: 229-17201 in U.S. Pat. No. 11,685,924. In some embodiments, exemplary sequences encoding IRESs for use in the RNA molecules of the present disclosure are all or a portion of the IRES sequence from encephalomyocarditis virus (EMCV). In some embodiments, exemplary sequences encoding IRES have 70-99% homology to the IRES sequence from encephalomyocarditis virus (EMCV). In some embodiments, the IRES sequence is a portfion of the IRES sequence from EMCV. In some embodiments, the portion of the IRES sequence from EMCV is 60-99% of the nucleotide length of EMCV. In some embodiments, the portion of the IRES sequence from EMCV is 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% the full nucleotide length of the IRES sequence from EMCV. In some embodiments, the EMCV sequence has the following sequence:

(SEQ ID NO: 18)
GAGGGCCCGGAAACCUGGCCCUGUCUUCUUGACGAGCAUUCCUAGGGGU
CUUUCCCCUCUCGCCAAAGGAAUGCAAGGUCUGUUGAAUGUCGUGAAGG
AAGCAGUUCCUCUGGAAGCUUCUUGAAGACAAACAACGUCUGUAGCGAC
CCUUUGCAGGCAGCGGAACCCCCCACCUGGCGACAGGUGCCUCUGCGGC
CAAAAGCCACGUGUAUAAGAUACACCUGCAAAGGCGGCACAACCCCAGU
GCCACGUUGUGAGUUGGAUAGUUGUGGAAAGAGUCAAAUGGCUCACCUC
AAGCGUAUUCAACAAGGGGCUGAAGGAUGCCCAGAAGGUACCCCAUUGU
AUGGGAUCUGAUCUGGGGCCUCGGUGCACAUGCUUUACAUGUGUUUAGU
CGAGGUUAAAAAACGUCUAGGCCCCCCGAACCACGGGGACGUGGUUUUC
CUUUGAAAAACACGAUGAUAA.

In some embodiments, the modular RNA carrier systems comprise an enhancer. In some embodiments, the enhancer can bind with the RNA binding protein sequence which comprises the eukaryotic initiation factor 3 (elF3) protein complex. The eIF-3 protein complex associates with the 40S ribosome and facilitates the recruitment of eIF-1, eIF-1A, eIF-2: GTP: methionyl-tRNAi and eIF-5 to form the 43S pre-initiation complex (43S PIC). The eIF-3 complex stimulates mRNA recruitment to the 43S PIC and scanning of the mRNA for AUG recognition. The eIF-3 complex is also required for disassembly and recycling of post-termination ribosomal complexes and subsequently prevents premature joining of the 40S and 60S ribosomal subunits prior to initiation. The eIF-3 complex specifically targets and initiates translation of a subset of mRNAs involved in cell proliferation, including cell cycling, differentiation and apoptosis, and uses different modes of RNA stem-loop binding to exert either translational activation or repression.

In some embodiments, the enhancer is a 5′ untranslated region (UTR) and/or a 3′UTR, wherein either or both may independently contain one or more different nucleoside modifications. In such embodiments, nucleoside modifications may also be present in the translatable region. Also provided are nucleic acids containing a Kozak sequence. Additionally, provided are nucleic acids containing one or more intronic nucleotide sequences capable of being excised from the nucleic acid.

The first terminal region may comprise a 5′ untranslated region (UTR) which may be the native 5′ UTR of the encoded polypeptide of interest or may be heterologous to the encoded polypeptide of interest. In one aspect, the 5′UTR may comprise at least one translation initiation sequence such as a kozak sequence, an internal ribosome entry site (IRES) and/or a fragment thereof. In some embodiments, the 5′UTR may comprise at least one fragment of an IRES.

Natural 5′ UTRs are involved in translation initiation. In some embodiments, natural 5′ UTR's comprise Kozak sequences. Kozak sequences can include the consensus CCR (A/G) CCAUGG, where R is a purine (adenine or guanine) three bases upstream of the start codon (AUG), which is followed by another ‘G’.

In some embodiments, the 5′ (or 3′ UTR) UTRs of this disclosure can include introns or portions incorporated into the flanking regions of the nucleic acids or mRNA of the invention.

### Synthesis of Engineered Derivatives

In some embodiments, this disclosure provides for mRNA comprising modified nucleic acids. Nucleic acids for use in accordance with the present disclosure may be prepared according to any available technique including, but not limited to chemical synthesis, enzymatic synthesis, which is generally termed in vitro transcription, enzymatic or chemical cleavage of a longer precursor, etc. Methods of synthesizing RNAs are known in the art (see, e.g., Gait, M. J. (ed.) Oligonucleotide synthesis: a practical approach, Oxford [Oxfordshire], Washington, D.C.: IRL Press, 1984; and Herdewijn, P. (ed.) Oligonucleotide synthesis: methods and applications, Methods in Molecular Biology, v. 288 (Clifton, N.J.) Totowa, N.J.: Humana Press, 2005; both of which are incorporated herein by reference).

Modified nucleic acids need not be uniformly modified along the entire length of the molecule. Different nucleotide modifications and/or backbone structures may exist at various positions in the nucleic acid. One of ordinary skill in the art will appreciate that the nucleotide analogs or other modification(s) may be located at any position(s) of a nucleic acid such that the function of the nucleic acid is not substantially decreased. A modification may also be a 5′ or 3′ terminal modification. The nucleic acids may contain at a minimum one and at maximum 100% modified nucleotides, or any intervening percentage, such as at least 5% modified nucleotides, at least 10% modified nucleotides, at least 25% modified nucleotides, at least 50% modified nucleotides, at least 80% modified nucleotides, or at least 90% modified nucleotides. For example, the nucleic acids may contain a modified pyrimidine such as uracil or cytosine. In some embodiments, at least 5%, at least 10%, at least 25%, at least 50%, at least 80%, at least 90% or 100% of the uracil in the nucleic acid is replaced with a modified uracil. The modified uracil can be replaced by a compound having a single unique structure, or can be replaced by a plurality of compounds having different structures (e.g., 2, 3, 4 or more unique structures). In some embodiments, at least 5%, at least 10%, at least 25%, at least 50%, at least 80%, at least 90% or 100% of the cytosine in the nucleic acid is replaced with a modified cytosine. The modified cytosine can be replaced by a compound having a single unique structure, or can be replaced by a plurality of compounds having different structures (e.g., 2, 3, 4 or more unique structures).

In some embodiments, this disclosure provides for mRNA encoding a peptide or polypeptide. Generally, the shortest length of a modified mRNA of the present disclosure can be the length of an mRNA sequence that is sufficient to encode for a dipeptide. In another embodiment, the length of the mRNA sequence is sufficient to encode for a tripeptide. In another embodiment, the length of an mRNA sequence is sufficient to encode for a tetrapeptide. In another embodiment, the length of an mRNA sequence is sufficient to encode for a pentapeptide. In another embodiment, the length of an mRNA sequence is sufficient to encode for a hexapeptide. In another embodiment, the length of an mRNA sequence is sufficient to encode for a heptapeptide. In another embodiment, the length of an mRNA sequence is sufficient to encode for an octapeptide. In another embodiment, the length of an mRNA sequence is sufficient to encode for a nonapeptide. In another embodiment, the length of an mRNA sequence is sufficient to encode for a decapeptide.

Examples of dipeptides that the modified nucleic acid sequences can encode for include, but are not limited to, carnosine and anserine.

In a further embodiment, the mRNA is greater than 30 nucleotides in length. In another embodiment, the RNA molecule is greater than 35 nucleotides in length. In another embodiment, the length is at least 40 nucleotides. In another embodiment, the length is at least 45 nucleotides. In another embodiment, the length is at least 55 nucleotides. In another embodiment, the length is at least 60 nucleotides. In another embodiment, the length is at least 60 nucleotides. In another embodiment, the length is at least 80 nucleotides. In another embodiment, the length is at least 90 nucleotides. In another embodiment, the length is at least 100 nucleotides. In another embodiment, the length is at least 120 nucleotides. In another embodiment, the length is at least 140 nucleotides. In another embodiment, the length is at least 160 nucleotides. In another embodiment, the length is at least 180 nucleotides. In another embodiment, the length is at least 200 nucleotides. In another embodiment, the length is at least 250 nucleotides. In another embodiment, the length is at least 300 nucleotides. In another embodiment, the length is at least 350 nucleotides. In another embodiment, the length is at least 400 nucleotides. In another embodiment, the length is at least 450 nucleotides. In another embodiment, the length is at least 500 nucleotides. In another embodiment, the length is at least 600 nucleotides. In another embodiment, the length is at least 700 nucleotides. In another embodiment, the length is at least 800 nucleotides. In another embodiment, the length is at least 900 nucleotides. In another embodiment, the length is at least 1000 nucleotides. In another embodiment, the length is at least 1100 nucleotides. In another embodiment, the length is at least 1200 nucleotides. In another embodiment, the length is at least 1300 nucleotides. In another embodiment, the length is at least 1400 nucleotides. In another embodiment, the length is at least 1500 nucleotides. In another embodiment, the length is at least 1600 nucleotides. In another embodiment, the length is at least 1800 nucleotides. In another embodiment, the length is at least 2000 nucleotides. In another embodiment, the length is at least 2500 nucleotides. In another embodiment, the length is at least 3000 nucleotides. In another embodiment, the length is at least 4000 nucleotides. In another embodiment, the length is at least 5000 nucleotides, or greater than 5000 nucleotides.

In some embodiments, amino acid sequence modifications are introduced by making nucleotide changes to DNA or RNA constructs encoding the peptides or proteins (including antibodies) described herein. In some embodiments amino acid sequence modifications to the peptides or proteins are introduced by peptide synthesis. Such modifications include, for example, deletions from, and/or insertions into and/or substitutions of, residues within the amino acid sequences. Any combination of deletion, insertion, and substitution can be made to arrive at the final construct, provided that the final construct possesses the desired characteristics. The amino acid alterations may be introduced in the ASPH antibody or ADAM10 substrate sequence at the time that sequence is synthesize or cloned.

Amino acid sequence insertions include amino- and/or carboxyl-terminal fusions ranging in length from one residue to polypeptides containing a hundred or more residues, and intrasequence insertions of single or multiple amino acid residues. Examples of terminal insertions include an antibody with an N-terminal methionyl residue or the antibody fused to a cytotoxic polypeptide. Other insertional variants of the antibody molecule include the fusion to the N- or C-terminus of the antibody to an enzyme (e.g. for ADEPT) or a polypeptide which increases the serum half-life of the antibody.

Another type of derivative is a one comprising one or more amino acid substitutions. Conservative substitutions are shown in Table 1 under the heading of “preferred substitutions.” In addition, exemplary substitutions noted in the table may be made made, or conservative or non-conservative substitutions, as further described below in reference to amino acid classes, may be introduced and the products screened.

TABLE 1
Conserved amino acid substitutions.

| | | Preferred |
|---|---|---|
| Original | Exemplary | Residue |
| Ala (A) | Val; Leu; Ile | Val |
| Arg (R) | Lys; Gln; Asn | Lys |
| Asn (N) | Gln; His; Asp, Lys; | Gln |
| | Arg | |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; | |
| | Leu Phe; Norleucine | |
| Leu (L) | Norleucine; Ile; Val; | |
| | Met; Ala; Phe | |
| Lys (K) | Arg; Gln; Asn | Arg |
| Met (M) | Leu; Phe; Ile | |
| Leu (F) | Trp; Leu; Val; Ile; Ala; | Tyr |
| | Tyr | |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Leu | |
| | Ala; Norleucine | |

In some embodiments, modifications in the biological properties of the peptides described herein are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Amino acids may be grouped according to similarities in the properties of their side chains (in A. L. Lehninger, in Biochemistry, second ed., pp. 73-75, Worth Publishers, New York (1975)):

Alternatively, naturally occurring residues may be divided into groups based on common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Conservative substitutions refer to substituting an amino acid in one class for another amino acid in the same class. Non-conservative substitutions refer to substituting a member of one of these classes for another class. Such substituted residues also may be introduced into the conservative substitution sites or, into the remaining (non-conserved) sites.

One type of substitutional derivative involves substituting one or more residues in the variable region or CDRs of a canonical antibody, nanobody (e.g. a camellid antibody comprising a variable heavy chain homodimer) or synthetic nanobody (sybody), which may be fusion of one or two nanobodies with an antibody Fc region, or a venus YFP peptide). Generally, the resulting derivatives(s) selected for further development will have modified (e.g., improved) biological properties relative to the parent antibody/nanobody/sybody from which they are generated. A convenient way for generating such substitutional variants involves affinity maturation using phage display.

In some embodiments, peptides corresponding to any of proteins or peptides of this disclosure, or regions of interest therein are substituted to generate all possible amino acid substitutions at each site. In some embodiments, the derivative proteins or peptides thus generated are displayed from filamentous phage particles as fusions to at least part of a phage coat protein (e.g., the gene III product of M13) packaged within each particle. The phage-displayed variants are then screened for their biological activity (e.g. binding affinity) as herein disclosed. In order to identify candidate sites for modification. Scanning (e.g., alanine scanning) can be performed to identify residues contributing significantly to binding. Alternatively, or additionally, it may be beneficial to analyze a crystal structure of the binding complex to identify contact points, for example: contact points between an antibody/nanobody, sybody or fusion protein and antigen, or contact points between the ASPH antibody and a receptor/decoy blocker, or derivatives of any of the foregoing as described herein. Such contact residues and neighboring residues are candidates for substitution according to techniques known in the art, including those elaborated herein. Once such derivatives/variants are generated, the panel of derivatives is subjected to screening using techniques known in the art, including those described herein, and antibodies with superior properties in one or more relevant assays may be selected for further development.

Nucleic acid molecules encoding amino acid sequence variants of the antibody are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody.

Diagnostic agents include imaging agents, e.g., fluorophores, radioisotopes, chemiluminescent, and colorimetric indicators.

As used herein, the term “therapeutic agent” includes agents that provide a therapeutically desirable effect when administered to subject. The agent may be of natural or synthetic origin. For example, it may be a nucleic acid, a polypeptide, a protein, a peptide, a radioisotope, saccharide or polysaccharide or an organic compound, such as a small molecule. The term “small molecule” includes organic molecules having a molecular weight of less than about, e.g., 1000 daltons. In one embodiment a small molecule can have a molecular weight of less than about 800 daltons. In another embodiment a small molecule can have a molecular weight of less than about 500 daltons.

In some embodiments, the dual-function immunotherapeutic can further comprise an anti-pathogen therapeutic agent. In some embodiments, this disclosure provides for a composition comprising a dual-function immunotherapeutic comprising a RNA oligonucleotide carriers and an anti-pathogen therapeutic agent. In some embodiments, the anti-pathogen therapeutic agent is an anti-cancer agent as described herin, or combinations thereof.

siRNA/shRNA/mRNA Delivery

Tumor-specific RNAi for in vivo therapeutics requires a carrier which can protect shRNA from degradation, increase their tumor specific targeting and penetration of cell membrane to achieve efficient gene silencing. Anti-apoptotic proteins are mostly overexpressed in tumor cells against apoptosis. The experimental results described herein indicate that developing tandem shRNAs (e.g., “shRNA-1” and “shRNA-2”) which independently target the multi-antiapoptotic genes including mcl-1 and bel-xl in a rational combination can efficiently induce tumor cell death. Mcl-1 and Bcl-xl belong to the anti-apoptotic proteins of the Bcl-2 family, which are major signal transducers, regulators, and effectors for apoptosis through the mitochondria. Knockdown anti-apoptotic proteins, such as Mcl-1 and Bcl-xl (and also Bcl-w), induce cell apoptosis, which introduces tumor death (Carrington, E., Zhan, Y., Brady, J. et al. Anti-apoptotic proteins BCL-2, MCL-1 and Al summate collectively to maintain survival of immune cell populations both in vitro and in vivo. Cell Death Differ 24, 878-888 (2017). https://doi.org/10.1038/cdd.2017.30). In some embodiments, tandem shRNA based tumor therapeutics can be used against multi-antiapoptotic genes for efficient tumor cell targeting and delivery using human hepatocellular carcinoma (HCC) models. This representative targeting method can be applied to other tumor types, for multiple tumor therapeutics. Representative shRNA can include or exclude MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13).

Nucleic Acid Nanodevice Based shRNA Delivery

DNA or RNA-based nanodevices affords high specificity and efficiency for the delivery of siRNA and/or shRNA for cancer therapy. In some embodiments, this invention features a DNA or RNA nanodevice for shRNA delivery, where the device features a targeting moiety for a protein on a target cancer cell membrane, and a lock that can be cleaved open by a cell surface enzyme on the target cancer cell membrane, optionally, a cell surface enzyme on the target cancer cell membrane associated with, or in close proximity to the protein on the target cancer cell membrane which is bound by the targeting moiety.

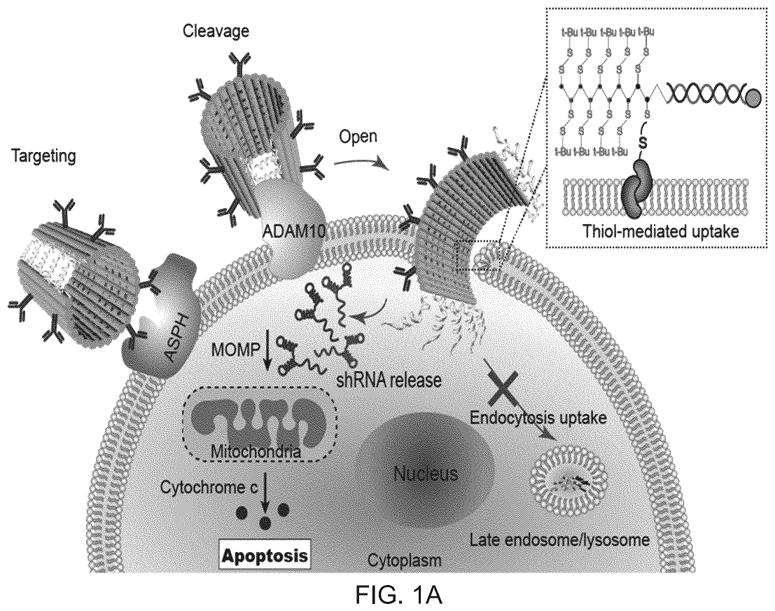

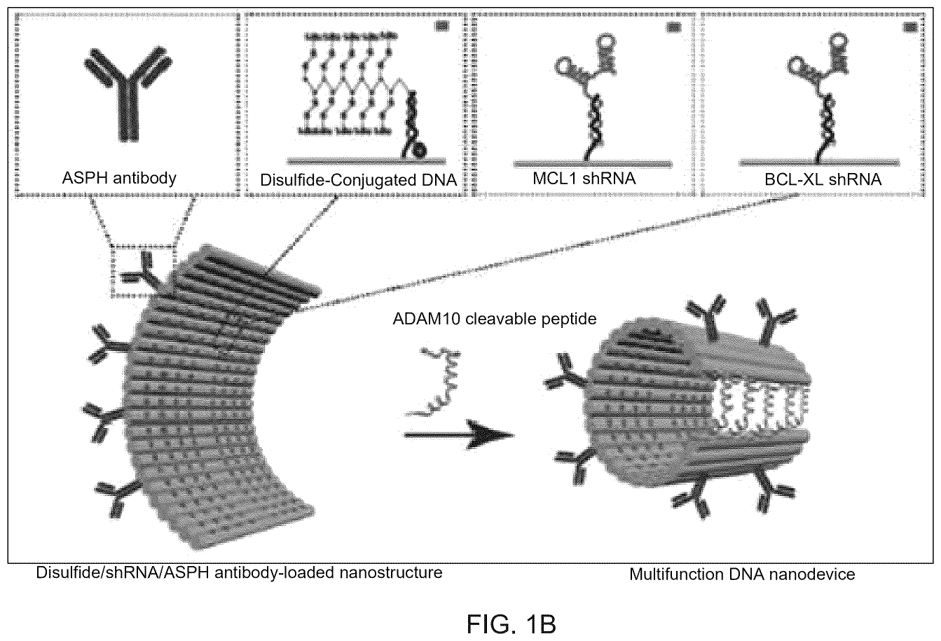

In some embodiments, the protein on the target cancer cell membrane is Aspartate beta-hydroxylase (ASPH). In one aspect, the targeting moiety is an antibody or fusion protein or fragments thereof, or a scfv or aptamer that selectively binds to ASPH. ASPH, a type II transmembrane protein, is highly expressed in HCC and in other tumors (e.g., breast, lung, colon, pancreatic, ovarian, and brain tumors) while maintaining low or negligible expression in normal tissue. ASPH generates enhanced cell motility, proliferation, invasion, and metastasis in HCC. Its over-expression predicts a poor prognosis with early disease reoccurrence and worse clinical outcome of patients in HCC. Several small molecule inhibitors, and antibodies against ASPH were independently developed to suppress tumor proliferation and metastasis in vitro and in vivo. It is therefore a well-recognized therapeutic target in cancer treatment (Kanwal, M., Smahel, M., Olsen, M. et al. Aspartate β-hydroxylase as a target for cancer therapy. J Exp Clin Cancer Res 39, 163 (2020); https://doi.org/10.1186/s13046-020-01669-w). The antibody against ASPH C-terminal extracellular domain is a targeting tool to specifically deliver a DNA nanostructure to the tumor site. In some embodiments, the lock for the nanodevice is a substrate for a Disintegrin and Metalloprotease Domain 10 (ADAM10) protein, which is a membrane-bound peptidase that cleaves extracellular portions of transmembrane proteins, and was discovered to physically interact with ASPH protein. ADAM10 is a transmembrane protease which is important for embryonic development and tissue homeostasis. The substrate peptide to the peptidase ADAM10 can be used as the lock of the Nucleic acid nanodevice. After targeting to tumor cells via ASPH antibodies, the Nucleic acid nanodevice opens up, exposing multi-disulfide units by which enables rapid cytosolic internalization (Yang et al., Disulfide-Containing Molecular Sticker Assists Cellular Delivery of DNA Nanoassemblies by Bypassing Endocytosis, Chinese Chem. Soc. Chem. 2020, 2, 1178-1186, DOI: 10.31635/ccschem.020.202000250), in response to the peptide cleavage by cell surface ADAM10 protein (see FIG. 1). Integrating multiple functional molecules on a representative Nucleic acid nanodevice of this disclosure as depicted in FIG. 1 facilitates the application of RNAi in tumor-targeting therapeutics.

In some embodiments, the protein on the target cancer cell membrane is Her2, wherein said target cells are Her2 positive cancer cells. In some embodiments, the Her2 positive cancer cells are identified by DNA sequencing and/or DNA genotyping. In some embodiments, the locking strand comprises a Her2 affibody to target Her2. In some embodiments, a locking strand comprises the MMP2 peptide. In some embodiments, the key strand comprises the MMP2 peptide. The MMP2 peptide can be conjugated to an oligonucleotide strand using selected oligonucleotide conjugation chemistry (IDT DNA, Inc.).

In some embodiments, the DNA nanodevice for targeted delivery of shRNA into a tumor comprises: a foldable dsDNA nanostructure in the shape of a sheet, a targeting moiety, e.g., an ASPH-targeting moiety (which can include or exclude an ASPH-antibody, aptamer, or binding portion thereof), a disulfide-conjugated DNA, and one or a plurality of shRNA sequences (which can include or exclude MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13), and an enzyme substrate, e.g., an ADAM10 cleavable peptide (which can include or exclude an ADAM10 peptidase substrate). A representative embodiment of the components of a DNA nanodevice for targeted shRNA delivery is depicted in FIG. 1B.

shRNAs Against Mcl-1 and Bcl-Xl Induce HCC Cell Death

In some embodiments, the Nucleic acid nanodevices of this disclosure deliver shRNAs to within a cell, and the shRNAs exhibit a selective cytotoxic effect. The shRNAs can act as a payload as a Nucleic acid nanodevice components. The shRNAs can be covalently or noncovalently connected to a DNA nanostructure (e.g., tube, sheet, triangle, etc.). The shRNAs can be confined to the Nucleic acid nanodevice when the Nucleic acid nanodevice is in a closed tubular format, and then released from the Nucleic acid nanodevice when the Nucleic acid nanodevice is in an open planar format, as per the condition-triggered Nucleic acid nanodevices described herein.

### Disulfide Units Enables Rapid Cytosolic Uptake of DNA Nanostructure

Nucleic acid nanodevice-based oligonucleotide delivery systems can be readily uptaken by cells without transfection reagents through the endocytosis pathway. However, endocytosis-independent pathways are preferred for cellular uptake because most clinical therapeutic oligonucleotides function in the cytoplasm. Disulfide units can trigger the cytosolic uptake of single-stranded oligonucleotide within 10 minutes (Z. Shu, I. Tanaka, A. Ota, D. Fushihara, N. Abe, S. Kawaguchi, K. Nakamoto, F. Tomoike, S. Tada, Y. Ito, Y. Kimura, H. Abe, Angew. Chem. Int. Ed. 2019, 58, 6611). This approach was used in the DNA nanostructure delivery systems of this disclosure and produced promising results.

Targeted siRNA Delivery Via Lipid Nanoparticles

In some embodiments, this disclosure provides for a lipid nanoparticle (LNP) encapsulating one or a plurality of siRNA oligonucleotides for targeted drug delivery carriers which enable clinical translation of gene therapies. The use of LNPs for drug delivery is disadvantaged by their lack of specificity. Most LNPs are localized in the liver tissue for hepatic gene therapy. Described herein is a modified LNP with targeting molecules anchored on its exterior surface which increase the siRNA delivery specificity for other tissues, including solid tumors.

### DNA Nanostructure Assisted Targeted Delivery of LNP

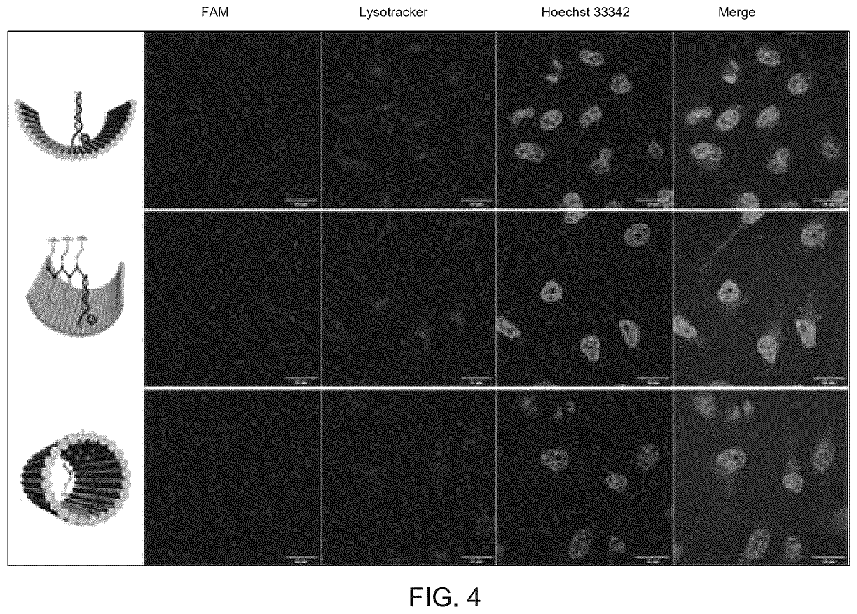

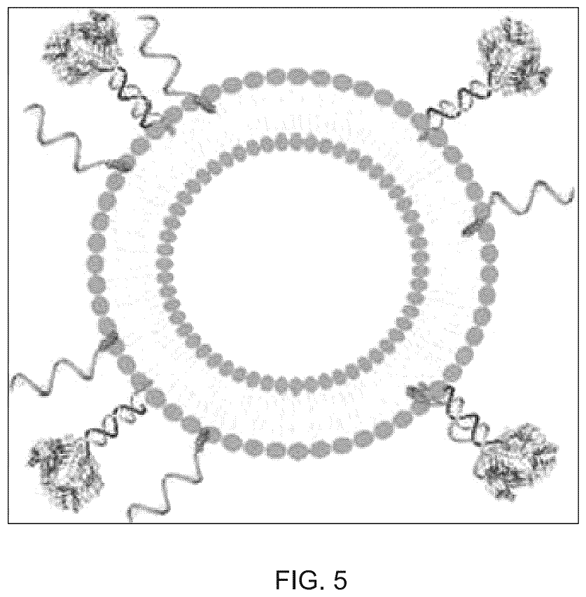

Membrane anchor molecules can be fused with DNA oligonucleotides (through common chemical conjugation methods) to load a chemically modified DNA oligonucleotide onto the LNP surface. FIG. 5 depicts one representative embodiment of a lipid nanoparticle of this disclosure. In some embodiments, the membrane anchor molecule can include or exclude: cholesterol, a fatty acid alcohol, or a fatty acid ether. In some embodiments, the fatty acid alcohol can include or exclude: dodecanol, tetradecanol, and octadecanol. In some embodiments, the modified DNA oligonucleotide can be 3′- or 5′ modified with a common modifier (e.g., 6-amino-modifier from IDT). Such design allows for the hybridization of its complimentary DNA oligonucleotides with the conjugated targeting molecules which can include or exclude proteins, peptides, and aptamers. Such modification enables the targeting properties of LNP.

Cholesterol-modified oligonucleotides were purchased from Integrated DNA Technologies Inc. (IDT, USA) with PAGE purification. The oligonucleotide strand was dissolved in water to make a 100 uM solution. The solution was quickly made into 5, 10, and 20 ul aliquots and lyophilized, so as to avoid aggregation of the cholesterol-modified strands in water. The LNP was incubated with 2X excess concentration of cholesterol-modified strands at 37 DC for 12 hours. The cholesteral-modified oligonucleotide had the following DNA sequence:

(SEQ ID NO: 304)
GTGTGCGGTATGGTATTTGATTCACCT-[TEG-Cholesterol
mod].

### DNA Nanostructure Assisted Targeted Delivery of LNP

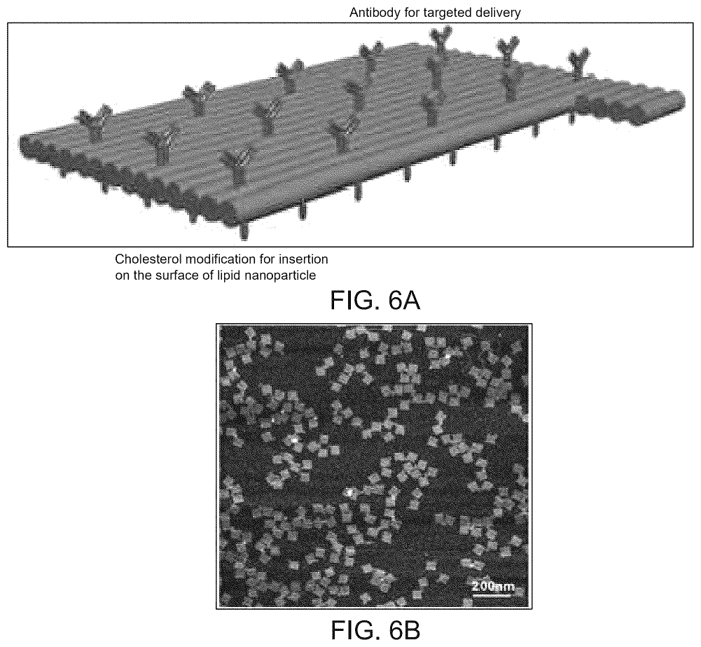

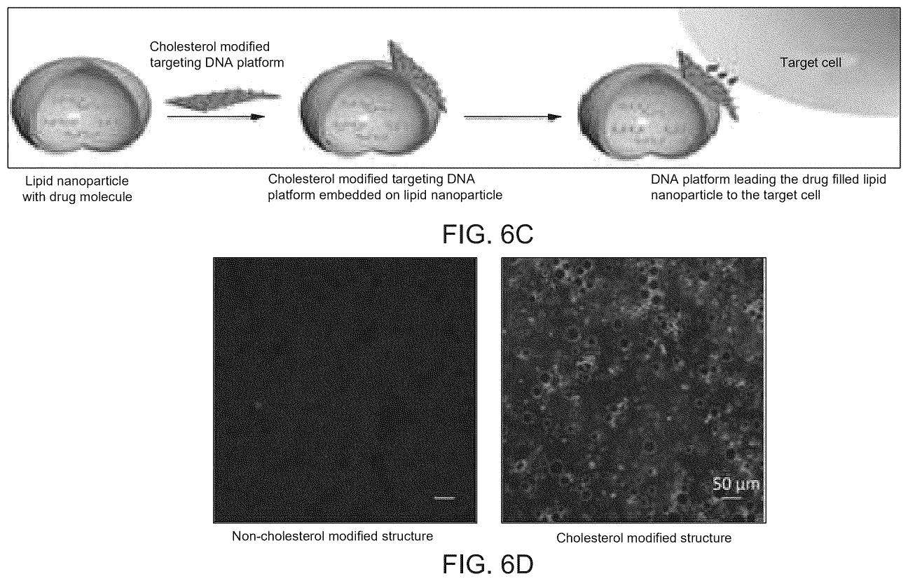

DNA nanostructures can be embedded onto the surface of LNPs. In the design of the nucleic acid nanodevices described herein, the targeting molecules (e.g. antibody) can be attached on a first surface of any of the DNA nanostructures of this disclosure, while the membrane-binding modifications (which can include or exclude cholesterol, decanol, dodecanol, tetradecanol, and octadecanol) are introduced onto a second surface of the DNA nanostructures to be embedded on the surface of an LNP loaded with a drug (e.g., biologic drug, small molecule drug, therapeutic RNA). The DNA nanostructures of this disclosure comprise loops and linker regions. The DNA nanostructures loaded LNP allows for a specific delivery of the LNPs and their contents to the target cells (FIG. 6). Multiple targeting molecules can be precisely organized on the nanostructure surface for multivalent targeting (FIG. 6A), which increases the targeting efficiency. The cholesterol modified DNA nanostructures efficiently fused onto the lipid surface, as demonstrated by using small unilamella liposomes/vesicles (SUVs) as a model (FIG. 6D), whereas LNPs without cholesterol did not fuse to the lipid surface (FIG. 6D).

In some embodiments, the DNA nanostructure platform comprises the DNA sheet described in U.S. Patent Application Publication No. US20190240248, herein incorporated by reference. In some embodiments, the DNA nanostructure platform comprises:
a single stranded DNA scaffold strand of about 5,000 to 10,000 bases in length;
a plurality of staple strands of DNA, wherein each staple strands are about 20 to 40 bases in length, wherein each staple strand has a unique sequence and is hybridized to a specific position on the DNA scaffold strand, wherein the plurality of staple strands hybridized to the DNA scaffold form a sheet having a top surface and a bottom surface; and one or more fastener strands of DNA, wherein the one or more fastener strands of DNA is capable of fastening the sheet into an nanostructure structure.

In some embodiments, the DNA nanostructure platform further comprises one or more DNA targeting strands, wherein each targeting strand is operably linked to a targeting moiety selected from an antibody to a cancer cell surface-expressing protein, a lipid bilayer anchor, or an oligonucleotide conjugated to a cancer cell surface-expressing protein or a lipid bilayer anchor.

Intelligent shRNA Delivery Platform

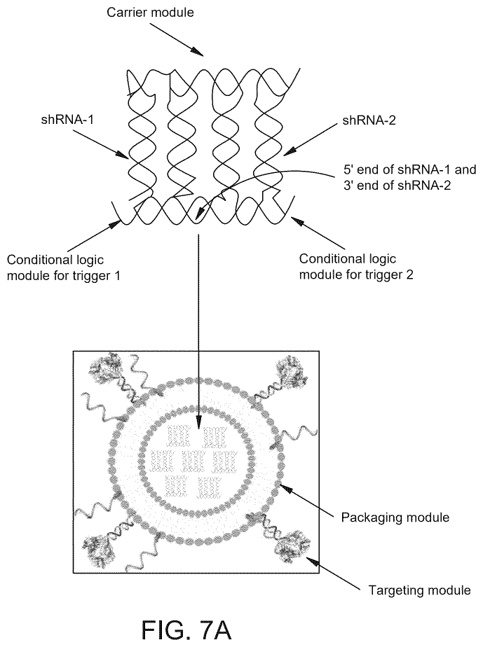

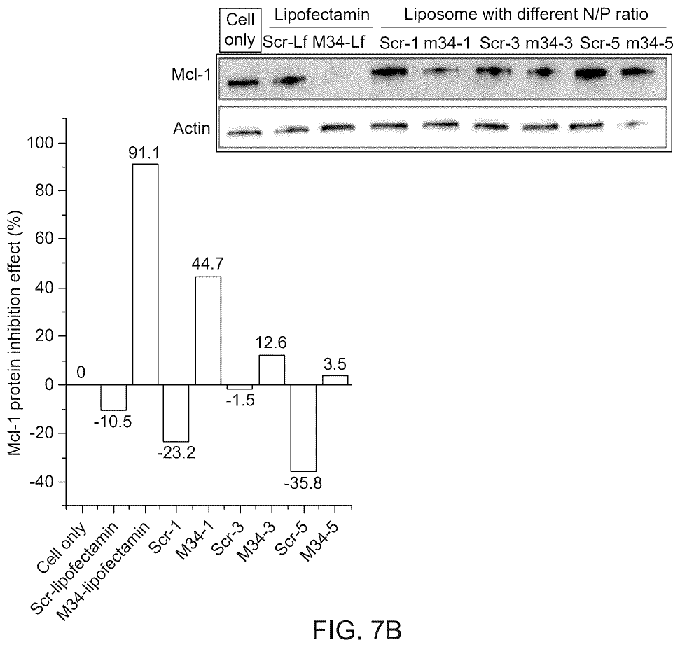

In some embodiments, this disclosure provides for a shRNA delivery platform with a smart release mechanism involving a targeted LNP delivery agent (FIG. 7A and FIG. 7B). Two heterodouble shRNAs can be connected together with trigger RNA molecules attached to loops and linker regions which are present in some embodiments of the DNA nanostructure delivery device. The trigger RNAs are antisense RNA molecules against tumor cell specific mRNAs. In some embodiments, the tumor cell specific mRNA is the ASPH mRNA. The shRNA molecules are only released when the cellular mRNA hybridizes with the trigger. Such intelligent shRNA delivery platform has the following advantageous features: 1) Selective and fast cellular uptake to release shRNA only to cytoplasm; 2) Controlled stoichiometry of multifunctional shRNA payload for optimal therapeutic effects; 3) Smart shRNA release with conditional molecular trigger and/or molecular computer to ensure therapeutic accuracy; and 4) Adaptable to lipid-based delivery technology with added benefits of precise targeting.

Multivalent siRNAs Design

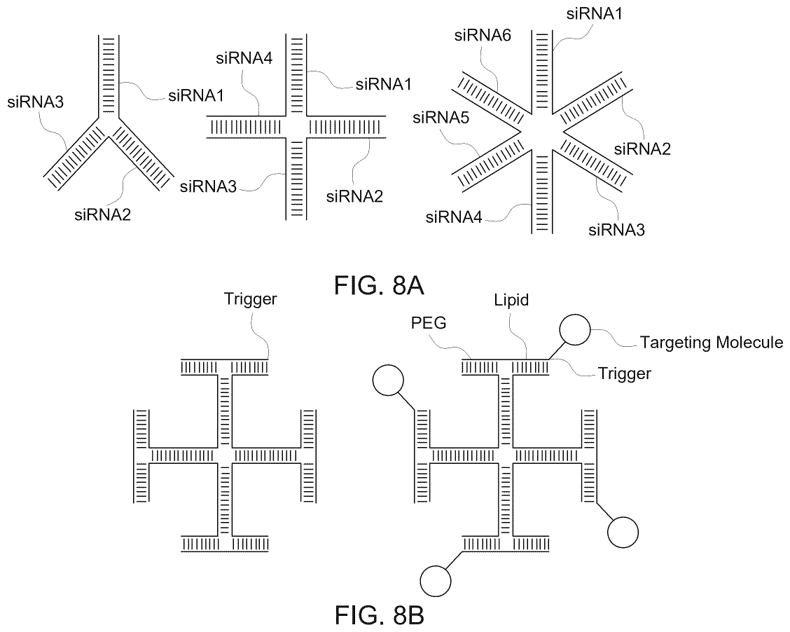

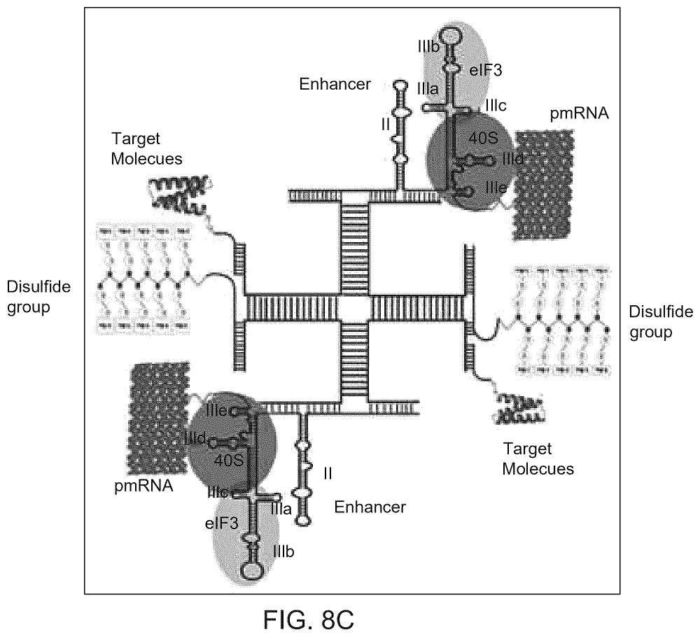

In some embodiments, this disclosure provides for a multivalent siRNA design which simultaneously delivers a various number of siRNA molecules into the target cells. Different numbers of RNA molecules can be connected to form junction structures (FIG. 8A), with each arm capable of being processed to be an siRNA molecule by the Dicer enzyme. Stoichiometry of siRNAs targeting different genes can be well controlled for optimal therapeutic effects. Furthermore, trigger RNA molecules can be introduced to protect the free ends of the junction design, allowing selective release of the siRNAs upon binding with target mRNA molecules (FIG. 8B). Polyethylene glycol (PEG) and/or lipid modification can be introduced to further increase the cellular stability (Schroeder, A., Levins, C.G., Cortez, C., Langer, R. and Anderson, D.G. (2010), Lipid-based nanotherapeutics for siRNA delivery. Journal of Internal Medicine, 267:9-21). Such multivalent siRNAs design can also be adapted to the targeted LNP delivery system for precise targeting.

In some aspects, the conjugation chemistry to connect an oligonucleotide with a peptide or protein can involve a heterobifunctional crosslinker. The heterobifunctional crosslinker can link a lysine or cysteine residue on a protein, polypeptide, or antibody (or sybody) to a polynucleic acid. In some aspects, the protein, polypeptide, or antibody (sybody) can comprise a glycine which can be treated with periodate to generate an aldehyde functionality which can be reacted with an amine or hydrazine via a heterobifunctional crosslinker. In some aspects, the heterobifunctional cross-linker can be selected from: EDC, SMCC, Sulfo-SMCC, MBS, Sulfo-MBS, SMPB, Sulfo-SMPB, GMBS, Sulfo-GMBS, SANH, C6-SANH, SHTH, SFB, C6-SFB, SIA, SIAB, sulfo-SIAB, SPDP, sulfo-SPDP, C6-SPDP, PDPH, and other heterobifunctional crosslinkers known in the art. In some aspects, the lysine can be reacted with a N-hydroxy-succinimide (NHS) moiety which is linked to an appropriate functional group which reacts with a modified polynucleotide. In some aspects, the cysteine can be reacted with a maleimide which is linked to an appropriate functional group which reacts with a modified polynucleotide. In some aspects, the modified polynucleotide can be a modified UTP nucleoside. In some aspects, the modified UTP nucleoside can be 5-DBCO-PEG4-UTP. In some aspects, the 5-DBCO-PEG4-UTP can be linked to an azido-functionalized protein. A protein, polypeptide, or antibody (or sybody) can be azido-functionalized via the reaction of a lysine residue with NHS-azide (Thermo Fisher), via reaction conditions well-known in the art.

Programmed mRNA (Pro-mRNA) Nanostructures

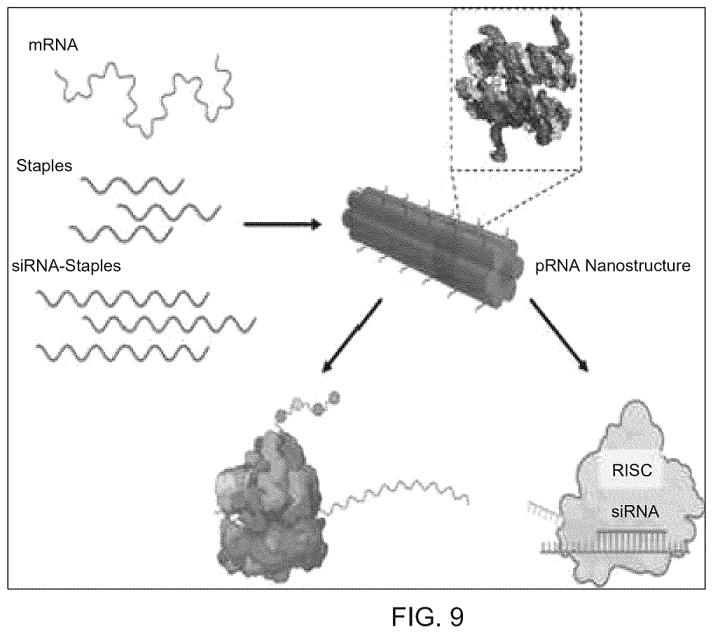

The inventors have developed programmed mRNA nanostructures which exhibit high stability and enable the simultaneous delivery of message RNA (mRNA) assembly nanostructure into the target cells. Applications include vaccine development when the mRNA is translated into proteins within the target cells. Selected mRNA sequences can be used to design and synthesize a programmed RNA nanostructure (FIG. 9), based on RNA self-assembly, including: RNA tiles, helix bundle, tetrahedron, square or rectangular sheet, and RNA origami using PX design. Stoichiometry of mRNA targeting different genes can be well controlled for optimal therapeutic effects. The compact Pro-mRNA nanostructures can increase the biostability of both mRNA by resisting nuclease from recognizing and binding RNA helix. After delivered into target cells, the Pro-mRNA nanostructure can release the mRNA. The mRNA can recruit ribosome and translate into targeting proteins for immunotherapeutic. Also, this Pro-mRNA nanostructure allows for the simultaneous delivery of a mRNA sequence and siRNA and/or shRNA sequences (when used as part of the staple strand sequences), which can facilitate control over the therapeutic effects. The Pro-mRNA nanostructures of this disclosure can provide a stable, efficient and pluripotent therapeutic method. Furthermore, trigger Pro-mRNA nanostructure can be translated to protein as an immunogenic neoantigens to enhancing cancer immunotherapy through the development of personalized mRNA nanostructure vaccines, to increase mRNA stability through the programmed mRNA design for delivery, allowing selective release of the mRNA nanostructure into cells.

### Design and Synthesis of Programmed RNA Nanostructure.

RNA is an ideal constructing material considering its innate ability to self-assemble into highly ordered structures through the simple Watson-Crick base pairing. In the past decade, a large number of RNA nanostructures have been designed and synthesized for fabricating synthetic architectures based on RNA self-assembly, including: RNA tiles, RNA single-stranded nanostructure and RNA wireframe nanostructure among many others. RNA nanotechnology provides a bottom-up engineered molecular toolbox to design, program, construct, and test multifunctional RNA nanostructures.

Highly Stable mRNA Vaccines

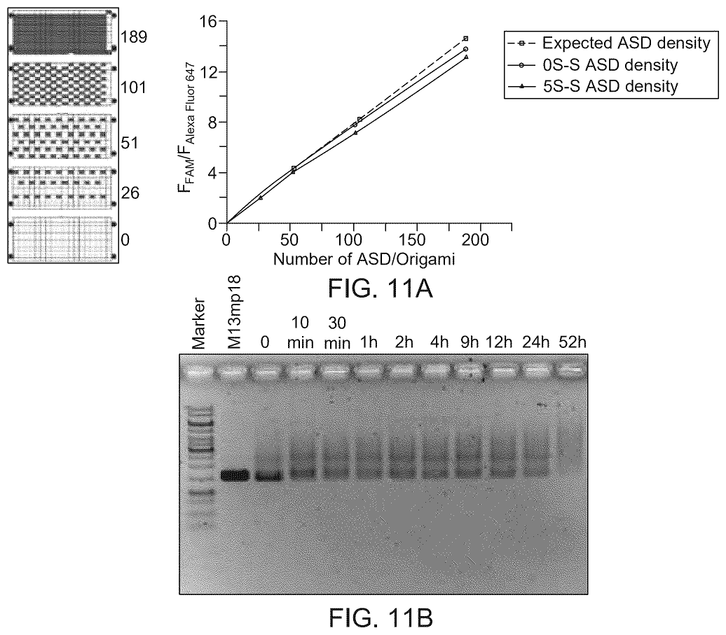

Conventional mRNA vaccines suffer from a stability issue—the mRNA sequences can be quickly degraded once released from their delivery vehicle (e.g., lipid nanoparticles for the case of the SARS-COV-2 mRNA-based vaccines. The fragility of mRNA vaccine result from two main factors. The first factor is the existence of 2′ hydroxyl group, which can induce alkaline hydrolysis of RNA strand. The deprotonated 2′ hydroxyl group of the ribose moiety will attack the nearby phosphate to form a pentacoordinate transition and break the RNA strand, which highly relies on a special conformation of RNA backbone of in-line 2′ hydroxyl group and 5′ oxyanion (FIG. 11A). Any RNA without a protection environment, however, has a very short lifetime due to the copious quantities of secreted non-specific RNases in the bloodstream (FIG. 11B). Described herein is a method of increasing the stability of mRNA vaccine to overcome these two shortcomings simultaneously. One or a plurality of mRNA strands can be folded into compact structures with staple RNA strands of this disclosure (FIG. 11C). The conformation of folded and stapled mRNA strands can be fixed to avoid the self-cleavage of the RNA backbones. Also, the compact mRNA nanostructures can hinder the recognition and reaction of RNases to increase the enzyme resistance of the mRNA vaccine based on these methods. Furthermore, the folded mRNA nanostructures allow for spatial control of the position of mRNA elements, which can include or exclude 5′ untranslation region (UTR) and 3′ UTR, to increase the efficiency of recruiting downstream translation complexes. In some embodiments, the 3′ UTR or 5′ UTR can be those described in U.S. Pat. Nos. 11,492,628, or 10080809, herein incorporated by reference.

In some embodiments, the programmed mRNA nanostructures comprise a long mRNA strand to carry out the translation template function and a plurality of RNA staple strands to fold mRNA into designed structure. The mRNA is designed with several structural elements, which can include or exclude: 5′cap, 5′/3′ UTR, Kozak sequence, polyA tail and open reading frame (ORF). The 5′ cap and polyA tail can increase the stability of the mRNA by preventing its degradation. The short 5′/3′ UTR from alpha fetoprotein (AFP) and Kozak sequence play important roles in recruiting ribosome to initialize the translation. The delivered mRNA strands will remain a single-stranded form to perform their biological functions once delivered into a cell. The open reading frame encoded for targeting protein is complementary with RNA staple strands and fold into designed structures.

In some embodiments, the ORF is contiguous with an Internal Ribosome Entry site. RNA binding proteins such as (eIF3) protein which protect the enhancer RNA sequence and provide the initiation of Pro-mRNA translation for protein. Modification with thiol group on the trigger RNA further enhances the cellular stability. The targeting molecule conjugation (e.g. GalNAc, affibody, small molecule drug) enables delivery specificity.

In some embodiments, this disclosure provides for a multivalent Pro-mRNA nanostructure which has a fourway juncture shape. The multivalient Pro-mRNA can include two Pro-mRNA with enhancers, two thiol groups, and two targeting molecules for delivery. The enhancer of Pro-mRNA can bind with eukaryotic initiation factor 3 (eIF3) to be a straightforward translation of Pro-mRNA to protein. Different mRNA can be designed and transcript to form the Pro-mRNA with the nanostructure. The multivalent Pro-mRNA nanostructure delivery is the target Pro-mRNA delivery and a straightforward protein translation. 1) Pro-mRNA increases the stability of mRNA, 2) enhancer increases protein translation of Pro-mRNA; 3) eIF3 protein protects enhancer against enzyme digest; 4) Thiol group mediates cellular uptake; 5) targeting molecules promote specific delivery.

The fourway junction structure can comprise four RNA oligonucleotides which form four arms. Each of the arms can further comprise a peripheral end, and the peripheral end comprises one or two non-complementary strands such as at least a portion of the arm comprises two separate single stranded RNAs. The two separate single stranded RNA sequences together can hybridize to a trigger RNA sequence, which the single stranded RNA further assembly with Pro-mRNA nanostructure sequence, wherein the Pro-mRNA sequence can translate protein expression in cancer cell by presenting a tumor associated antigen on the cancer cell membrane which promote an immune cell to recognize and kill the cancer cell. The tumor associated antigen can be selected from LIME1, RAC3, AP2S1, Survivin, CTSL, MPZL2, and LSP1 genes as representative examples (SEQ ID NOs: 22-28). The complementary trigger RNA sequence will be released by ribosome helicase, when the ribosome binds with the enhancer structure. The other trigger RNA sequence can be modified with a disulfide group as a membrane penetration enhancer, and a targeting molecule. In some embodiments, the targeting molecule can be selected from an affibody or portion thereof to ASPH or Her2, GalNAc, an anti-cancer agent as described herein, or an shRNA as described herein.

The programmed mRNA nanostructures comprise a long mRNA strand to carry out the translation template function and several RNA staples strands to fold mRNA into designed structure. The mRNA is specially designed with several structural elements, including enhancer, Kozak sequence, polyA (30nt) tail and open reading frame (ORF). In this design, the programmed mRNA nanostructures are composed of a long mRNA strand to carry out the enhancer and several RNA staples strands to fold mRNA into designed structure. The enhancer promote mRNA translate to protein and increase protein yield. The short 5′ Enhancer and Kozak sequence play important roles in recruiting ribosome to initialize the translation. These sequences will remain a single-stranded form to perform their biological functions. The open reading frame encoded for targeting protein will be complementary with RNA staple strands and fold into designed structures (see FIG. 8A, FIG. 8B, and FIG. 8C).

### Compositions and Kits

Certain embodiments of the invention also provide a composition comprising a small RNA carrier complex as described herein and a carrier. In certain embodiments, the composition comprises a plurality of RNA oligonucleotide carriers and a carrier. In certain embodiments, the composition further comprises at least one therapeutic agent as described herein. In certain embodiments, the composition is a pharmaceutical composition and the carrier is a pharmaceutically acceptable carrier.

The present invention further provides kits for practicing the present methods. Accordingly, certain embodiments of the invention provide a kit comprising a dual function immunotherapeutic or a multi-functional vaccine described herein and instructions for administering the therapeutic to treat a disease or condition.

### Certain Methods

Certain embodiments of the invention provide a method of presenting a RNA oligonucleotide molecule carrier to a tumor cell in a subject (e.g., a mammal, such as a human), comprising administering to the subject an effective amount of an RNA oligonucleotide molecule carrier or composition as described herein. The phrase “effective amount” means an amount of a RNA oligonucleotide molecule carrier described herein that elicits cancer cell death or apoptosis.

Certain embodiments of the invention also provide a method of treating a disease or disorder in a subject, comprising administering to the subject a therapeutically effective amount of an RNA oligonucleotide molecule carrier or a composition as described herein.

In certain embodiments, a method of the invention further comprises administering at least one anti-cancer agent to the subject.

The at least one therapeutic agent may be administered in combination with the dual-functional immunotherapeutic comprising a RNA oligonucleotide molecule carrier. As used herein, the phrase “in combination” refers to the simultaneous or sequential administration of the RNA oligonucleotide molecule carrier and the at least one anti-cancer agent. For simultaneous administration, the RNA oligonucleotide molecule carrier and the at least one anti-cancer agent may be present in a single composition or may be separate (e.g., may be administered by the same or different routes).

Certain embodiments of the invention provide a RNA oligonucleotide molecule carrier or a composition as described herein for use in medical therapy.

Certain embodiments of the invention provide the use of an RNA oligonucleotide molecule carrier or a composition as described herein for the manufacture of a medicament for treating a disease or disorder in a subject.

Certain embodiments of the invention provide the use of an RNA oligonucleotide molecule carrier or a composition as described herein for the manufacture of a medicament for treating a disease or disorder in a subject, in combination with at least one anti-cancer agent.

Certain embodiments of the invention provide an RNA oligonucleotide molecule carrier or a composition as described herein for the prophylactic or therapeutic treatment a disease or disorder.

Certain embodiments of the invention provide an RNA oligonucleotide molecule carrier or a composition as described herein for the prophylactic or therapeutic treatment of a disease or disorder, in combination with at least one anti-cancer agent.

### Administration

In some embodiments, methods of the invention comprise administering a dual functional immunotherapeutic or a multi-functional vaccine comprising a modular RNA carrier system described herein, and optionally, a therapeutic agent to a subject. Such compounds may be formulated as a pharmaceutical composition and administered to a mammalian host, such as a human patient in a variety of forms adapted to the chosen route of administration, i.e., orally or parenterally, by intravenous, intramuscular, intraperitoneal or topical or subcutaneous routes.

The modular RNA carrier system may be administered intravenously or intraperitoneally by infusion or injection. Solutions of the active compound or its salts is prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle is a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity is maintained, for example, by the formation of liposomes, by the maintenance of the required particle size in the case of dispersions or by the use of surfactants. In many cases, it will be preferable to include isotonic agents, for example, sugars, buffers or sodium chloride. Prolonged absorption of the injectable compositions is brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Pharmaceutical compositions for use in accordance with the present invention may be formulated using one or more physiologically acceptable carriers or excipients. Any suitable concentration of the modular RNA carrier systems may be used, and any active pharmaceutical ingredient will be administered in an amount effective to achieve its selected purpose. In some embodiments, the dosage can be from 10 to 50 micrograms. In some embodiments, the dosage can be administered one, two, three, four, or five times dailer. In some embodiments, the dosage can be administered once per day, once per week, once per two weeks, one per three weeks, once per four weeks, once per five weeks, or once per six weeks. In some embodiments, the dosage can be administered a total of one, two, three, four, five, six, seven, eight, or nine times.

A variety of suspending fluids or carriers may be employed to suspend the modular RNA carrier system. Such fluids include without limitation: sterile water, saline, buffer, or complex fluids derived from growth medium or other biological fluids. Preservatives, stabilizers and antibiotics may be employed in the compositions comprising the modular RNA carrier compositions described herein.

Methods of making a pharmaceutical composition include admixing at least one active compound or agent, as defined above, together with one or more other pharmaceutically acceptable ingredients, such as carriers, diluents, excipients, and the like. When formulated as discrete units, such as tablets or capsule or suspension, each unit contains a predetermined amount of the active compound or agent.

Suitable formulations will depend on the method of administration. The pharmaceutical composition is preferably administered by intradermal administration, but other routes of administration include for example oral, buccal, rectal, parenteral, intramuscular, subcutaneous, intraperitoneal, transdermal, intrathecal, nasal, intracheal. The polyvalent vaccine can also be administered to the lymph nodes such as axillary, inguinal or cervial lymph nodes. The active agent may be systemic after administration or may be localized by the use of regional administration, intramural administration, or use of an implant that acts to retain the active dose at the site of implantation.

Pharmaceutical compositions described herein may be administered directly, they may also be formulated to include at least one pharmaceutically-acceptable, nontoxic carriers of diluents, adjuvants, or non-toxic, nontherapeutic, fillers, buffers, preservatives, lubricants, solubilizers, surfactants, wetting agents, masking agents, and coloring agents. Also, as described herein, such formulation may also include other active agents, for example, other therapeutic or prophylactic agents, nonimmunogenic stabilizers, excipients and the like. The compositions can also include additional substances to approximate physiological conditions, such as pH adjusting and buffering agents, toxicity adjusting agents, wetting agents and detergents.

Adjuvants can include or exclude: polymers, copolymers such as polyoxyethylene-polyoxypropylene co-polymers, including block co-polymers; polymer P1005; monotide ISA72; Freund's incomplete adjuvant; sorbitan monooleate; squalene; CRL-8300 adjuvant; alum; QS 21, C34, muramyl dipeptide; trehalose; bacterial extracts, including mycobacterial extracts; detoxified endotoxins; membrane lipids; water-in-oil mixtures, water-in-oil-in-water mixtures or combinations thereof.

Useful dosages of compounds can be determined by comparing their in vitro activity, and in vivo activity in animal models. Methods for the extrapolation of effective dosages in mice, and other animals, to humans are known to the art; for example, see U.S. Pat. No. 4,938,949, herein incorporated by reference.

The compound may be conveniently formulated in unit dosage form. In one embodiment, the invention provides a composition comprising a compound formulated in such a unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

### Embodiments of the Invention

In certain embodiments, the present invention provides a pharmaceutical composition comprising the modular RNA carrier described herein and a pharmaceutically acceptable carrier.

In certain embodiments, the present invention provides the pharmaceutical composition described herein and further comprises at least one anti-cancer agent.

In certain embodiments, the present invention provides a method of treating a disease or disorder in a subject, comprising administering to the subject a therapeutically effective amount of a complex or a composition as described herein.

In certain embodiments, the present invention provides a method of slowing or suppressing cancer cell growth in a subject (e.g., a mammal, such as a human) as compared to a control subject, comprising administering to the subject an effective amount of a complex or a composition as described herein.

### EXAMPLES

The invention will now be illustrated by the following non-limiting Examples.

### Materials and Methods

All RNAs were transcribed in vitro using in-house T7 polymerase using DNA templates (Integrated DNA Technologies) containing the T7 promoter. Transcriptions were concentrated by ethanol precipitation and purified by gel extraction using 12.5-20% denaturing polyacrylamide gel electrophoresis. RNA was removed from gel fragments by electro-elution, concentrated by ethanol precipitation, resuspended in 20 mM sodium phosphate (pH=6.0), 100 mM KCl, 500 mM NaCl, and 1 mM EDTA buffer, then dialyzed into 20 mM sodium phosphate (pH=6.0), 0.01 mM EDTA for NMR data collection.

### Nucleic Acid Nanodevice Nanostructure Assembly

When the small RNA carrier system is a nucleic acid nanodevice comprising DNA (“DNA nanodevice”), the purified DNA molecule will be diluted to 20 nM in 1×PBS buffer (20 mM Sodium phosphate, 130 mM Sodium chloride, pH 7.4). The resulting solution will be annealed from 65° C. to 25° C. with a cooling ramp of 1° C. per 20 minutes to form the desired structures. The assemble DNA nanodevice will be concentrated to the desired concentration using an Amicon Ultra-0.5 mL centrifugal filter (Millipore, 100 kDa cut off).

### Atomic Force Microscope Characterization

Nucleic acid nanodevices will be imaged in “ScanAsyst mode in fluid,” using a Dimension FastScan microscope with PEAKFORCE-HiRs-F-A tips (Bruker Corporation). After annealing, 2 μl of each sample will be deposited onto a freshly cleaved mica surface (Ted Pella, Inc.), and left to adsorb for 1 minute. Then, 80 μl of 1× TAE-Mg buffer and 2 μl 100 mM of a NiCl₂ solution was added onto the mica, and 40 μl of the same buffer will be deposited onto the microscope tip. The samples will then be scanned by following the manufacturer's instructions.

### Animals

Female BALB/c mice will be obtained from Charles River Laboratories and maintained in a pathogen-free animal facility at the Arizona State University Animal Resource Center. All mice will be handled in accordance with the Animal Welfare Act and Arizona State University Institutional Animal Care and Use Committee (IACUC). Before experimental treatment, the mice will be randomly distributed in cages and allowed to acclimate for at least 1 week prior to vaccination.

DNA strands:

Single-stranded MI3mp18 DNA was purchased from New England Biolabs. Staple strand oligonucleotides were obtained from Integrated DNA Technologies (IDT) on 96-well plates and used without further purification. Thiol-modified DNA oligonucleotides were also purchased from IDT, and were purified by denaturing PAGE before use.

### Reagents:

N-Succinimidyl 3-(2-pyridyldithio) propionate (SPDP) and tris(2-carboxyethyl) phosphine (TCEP) were obtained from Pierce. Dimethyl sulfoxide (DMSO) was purchased from Sigma.

### Buffers:

Phosphate buffered saline (PBS), HEPES sodium salt, Tris buffered saline (TBS), Tris base, acetic acid, EDTA, and magnesium acetate were purchased from Sigma. 1× TAE/Mg2+ buffer (pH 8.0) is prepared by 40 mM Tris, 20 mM acetic acid, 2 mM EDTA and 12.5 mM magnesium acetate.

### Dye-Labeling Reagents:

NHS-Cy3, Cy5 amine reactive dyes were purchased from GE Healthcare Life Sciences. NHS-AlexaFluor®555 and AlexaFluor®647 amine reactive dyes were obtained from Life Technologies.

Amicon centrifugal filters were purchased from Millipore.

PEG 8000 was purchased from Promega.

### Surface PEGylating Reagents:

APTES (3-Aminopropyl) triethoxysilane was purchased from Sigma-Aldrich. mPEG-SV A 5k and biotin-PEG-SY A 5k were obtained from Laysan Bio, Inc.

### TEM Imaging:

TEM grids (400 mesh, copper grid coated with ultrathin carbon, Ted Pella) were glow discharged (Emitech KI OOX). 2 μl concentrated samples were deposited onto the grids for 1 min, washed with 10 μl DI water for 5 sec, stained with 10 u 1 1% uranyl formate twice (2 sec for the first time and 15 sec for the second time), and imaged using Philips CMI2 transmission electron microscope.

### Example 1. Assembly of Targeting Antibody to Nucleic Acid Nanodevice

DNA nanostructure technology allows a long DNA molecule or plurality of DNA oligonucleotides to be programmed to self-assemble into nucleic acid nanodevice that is uniformly dispersed, and structurally stable.

DNA-DBCO conjugates can be prepared to create a ssDNA which can hybridize to a ssDNA region on a DNA nanodevice (or ssRNA for the corresponding RNA nanodevice). In some embodiments, a targeting antibody can be modified to incorporate an azido-phenylalanide moiety which can react with the DNA-DBCO conjugate per the reaction scheme depicted below to create the antibody (protein)-ssDNA conjugate. DNA-DBCO conjugate covalently links with azF protein/peptide through a non-copper catalyzed click chemistry reaction.

### Example 2. Programmed mRNA Nanostructures

RNA nanostructure sequence design

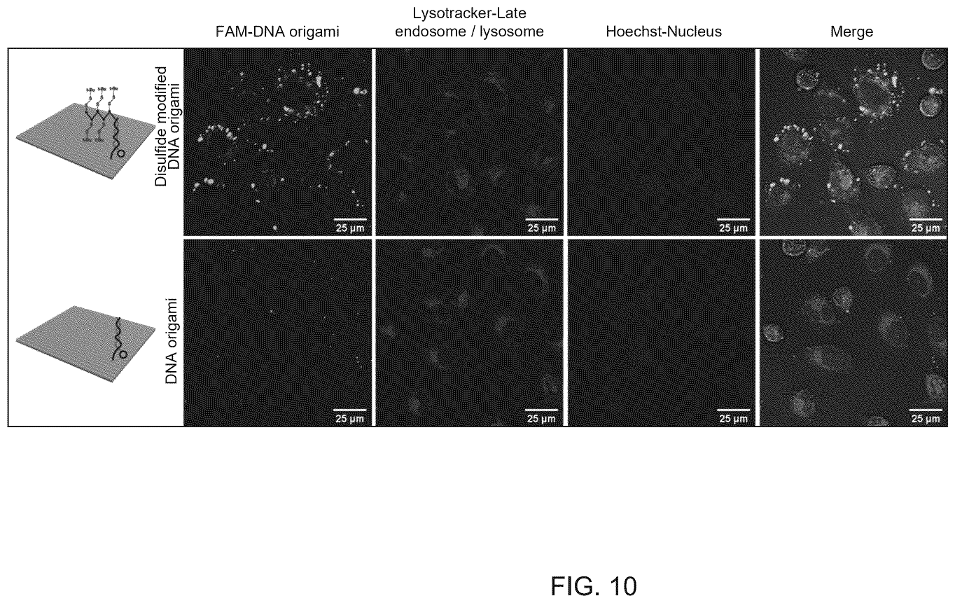

As shown in FIG. 10, RNA based six-helix bundle nanostructure was designed, synthesized and characterized. RNA nanostructure sequences were designed with the Tiamat software (Williams, S., et al., Tiamat: A three-dimensional editing tool for complex DNA structures, in International Workshop on DNA-Based Computers (Springer, 2008), pp. 90-101). Sequence generation follows the criteria in the software. After the sequences were generated, the T7 promoter sequences followed with two or three consecutive Gs were added to the ends to facilitate efficient in vitro transcription reactions.

### RNA Nanostructures Sample Preparation

The DNA templates for transcribing RNAs were cloned into pBluescriptII SK (+) vector with T7 promoter sequences and Hind III cleavage site on the 5′ end and 3′ end respectively, and ordered as gene synthesis products from Genscript Biotech Corp. The plasmids were transformed into E. coli competent cells. E. coli colonies were formed after overnight growth, and single colonies were picked up for overnight growth in LB medium. Single colonies were amplified the next day, and plasmids were purified. The final plasmids were linearized by Hind III, and transcribed by T7 RNA polymerase. The purified RNA in 50 nM was mixed with 1X TAE Mg2+ buffer (40 mM Tris, 20 mM acetic acid, 2 mM EDTA, and 12.5 mM magnesium acetate, pH 8.0) for folding. The steps for the annealing were as follows: 65° C. for 5 min; 0° C. for 5 min; 65° to 25° C. at −1° C. per 30 min. For AFM imaging, the sample (5 μl) was deposited onto a freshly cleaved mica surface (Ted Pella, Inc.) and left to adsorb for 1 min. A volume of 60 ul of 1× TAE-Mg2+ and 5 μl 200 mM NiCl₂ was added onto the mica. The samples were imaged in “ScanAsyst in Fluid” mode with a ScanAsyst-liquid+ tip on the Dimension FastScan AFM (Bruker).

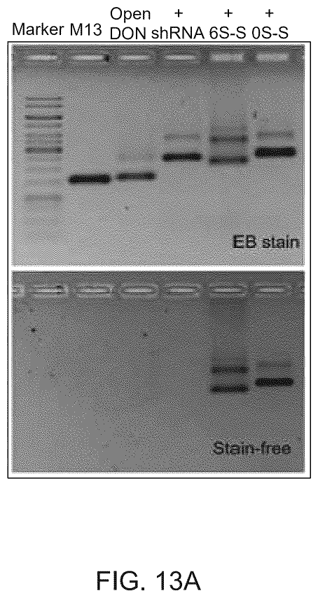

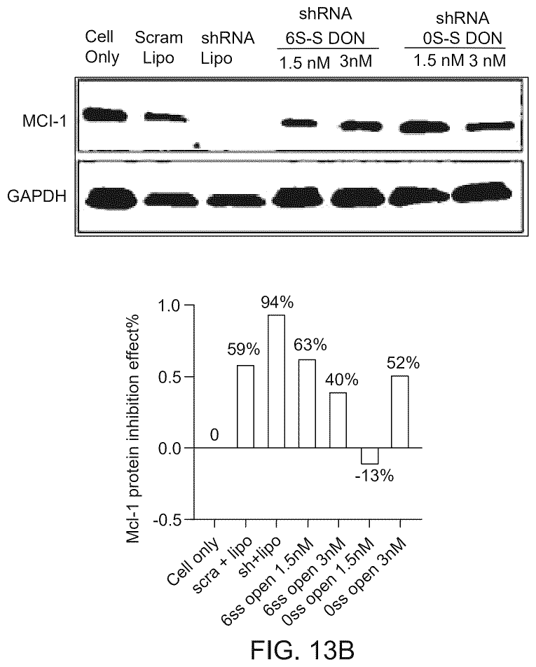

To validate the programmed mRNA nanostructures for bio-stability and transcription efficiency, a small fluorescent protein mCherry (236 residues) is selected as the target protein in the ORF of mRNA (711 nt) for the further validation of programmed mRNA vaccine strategy. First, the folded mRNA nanostructures will be applied into the in vitro translation to identify whether the folded structures can be denatured by the ribosome and act as the template for the translation. The in-solution stability against self-cleavage of the folded mRNA will be tested by mixing mRNA nanostructures with high pH buffer in a time gradient before translation and the fluorescence intensity of mCherry will be measured to indicate the stability of mRNA. Then, the mRNA nanostructures will be transfected into cells to express mCherry in vivo to valid the mRNA stability in a RNase environment. The confocal microscopy and flow cytometry will be used to monitor the time-varying expression level (FIG. 13).

### Example 3. Disulfide-Based Oligonucleotides for Rapid Cystolic Update of DNA Nanostructures (Nanaosheets)

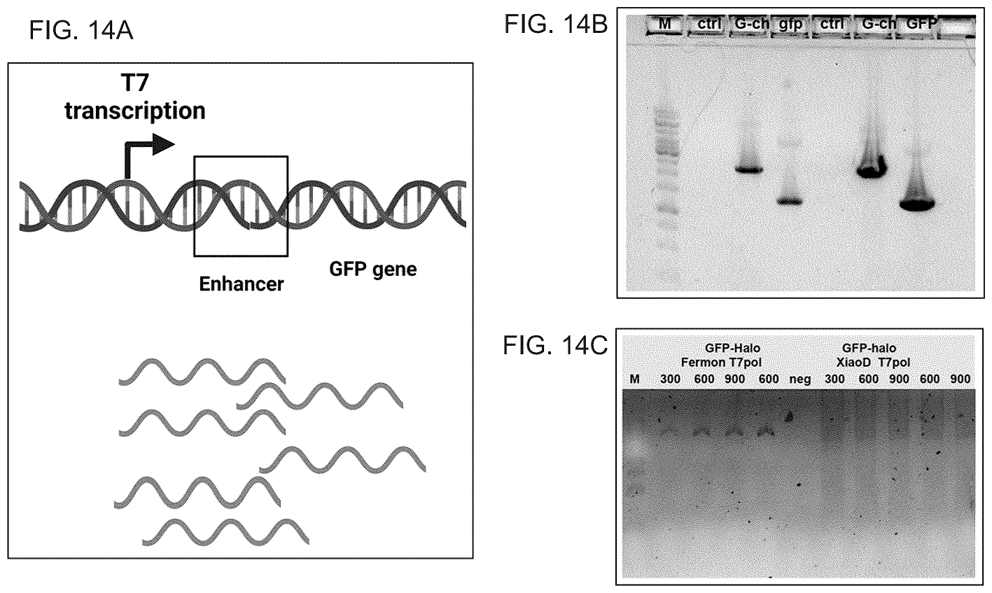

The cellular uptake was evaluated using disulfide modified DNA labeled with fluorescein by confocal microscope laser microscope (CLSM). HeLa cells were incubated with 3 nM disulfide modified DNA for 2 hr and used DNA nanosheets without disulfide units as control (FIG. 14). For DNA nanosheets without disulfide modification, loaded DNA strand was minimally adsorbed. In contrast, disulfide modified DNA nanosheets was significantly adsorbed into the DNA nanostructure. The intracellular distribution showed that DNA nanosheets with disulfide modification was not co-localized with late endosome/lysosome which indicates that disulfide modified sequences in the DNA nanosheets internalize into cells via a non-endocytic pathway. These results indicate that disulfide modification not only promotes cellular uptake of DNA nanosheets, but also enables fast cytosolic uptake, which is desirable for the delivery of cargos that require cytosol transportation.

In a separate experiment further establishing the disulfide-based sequences promote rapid cystolic update of nucleic acid nanodevices of this disclosure, HeLa cells were incubated for 1 hour with the 5 disulfide-units modified DNA nanosheets (FAM-5-DADO) (Disulfide-conjugated antisense DNA loaded DNA nanosheet) with FAM dye labeled were readily detected inside the cells (FIG. 4, central panel). Within only 5 minutes, the fluorophores were visualized within the cells, indicating the internalization is rapid. Furthermore, none of the FAM fluorescence overlapped with the lysotracker dye, which stains endosomes and lysosomes, indicating the FAM-5-DADO is not trapped inside the endosome. However, no FAM fluorescence from ADO was observed (FIG. 4, top panel). The FAM dye is pH sensitive, with weak fluorescence intensity under acidic pH, like endosome environment. This result correlates with the previous finding that nucleic acid nanodevices without disulfide units were trapped inside the endosomes. The closed FAM-5-DADO structure with disulfide-unit entrapped inside the cage was prepared to further confirm the functionality of the disulfide-unit. Much weaker FAM fluorescence was observed for the closed DNA nanodevice structure (FIG. 4, bottom panel) compared to the open cage configuration. These results indicate that the disulfide units promote rapid cytosolic uptake of DNA nanostructures.

### DNA Nanostructure Preparation and Purification.

DNA nanostructures were made by mixing a long scaffold strand (7249 bases) with a plurality of short staple strands. The staple strands included extended core strands (SEQ ID NOs: 29-132), open blocker strands (SEQ ID NOs: 133-148), close lock strands (SEQ ID NOs: 149-164), hairpin and 4T edge strands (SEQ ID NOs: 165-187), and optionally shRNA bald core strands (SEQ ID NO: 189-273). Oligonucleotides were acquired from IDT (USA) and combined in 1 × TAE-Mg buffer (40 mM Tris, 20 mM acetic acid, pH 8.0, 2 mM EDTA, 12.5 mM Mg (CH₃COOH)₂). The final concentrations of scaffold DNA and basic staple strands were 10 nM and 50 nM, respectively. The mixture was heated to 70° C., then annealed by cooling to 25° C. at a rate of 10 min/° C. using an Eppendorf thermal cycler.

The resulting DNA nanostructures were separated from excessive staple strands using Amicon Ultra-0.5 ml 100 kD centrifugal filters (Millipore Corporation, Bedford, USA). After purifying DNA nanostructures, then load with or without disulfide modified antisense DNA.

### Preparation of Disulfide Modified Antisense DNA

To a cooled (0-5° C.) solution containing 1,3-propanediol (19.02 g, 250 mmol, 20.0 eq.) in pyridine (75 mL) was added dropwise a solution containing DMT-Cl (4.23 g, 12.5 mmol) in pyridine (30 mL). The mixture was stirred at r.t. under argon for 18 h. The solvent was evaporated under diminished pressure and the residue was partitioned between water (120 mL) and EtOAc (100 mL). The organic phase was washed with water (50 mL), brine (50 mL), dried (MgSO5) and evaporated under diminished pressure. The residue was purified on a silica gel column (6×14 cm), eluting with 1:1 hexane-EtOAc. The product was obtained as yellow syrup: yield 4.40 g (93%). To a solution containing 3-mercaptopropanol (1.73 mL, 1.84 g, 20 mmol) and 2-methyl-2-propanethiol (22.4 mL, 18.0 g, 200 mmol) in absolute EtOH (10 mL) was added dropwise a solution containing iodine (5.08 g, 20 mmol) in EtOH (50 mL). The mixture was stirred under argon at r.t. for 18 h (pale yellow solution). The reaction was quenched by dropwise addition of satd. aq. NaHCO₃ (100 mL), stirred for 1 h then EtOH was evaporated under diminished pressure. The residue was suspended in EtOAc (150 mL) and washed successively with 10% aq. NaHSO3 (3×100 mL) and brine (100 mL). The organic layer was dried (MgSO4) and evaporated under diminished pressure. The residue was purified on a silica gel column (6×11 cm), eluting with 2:1 hexane-EtOAc (700 mL) and 1:1 hexane-EtOAc (300 mL). The disulfide was obtained as a pale yellow oil: yield 3.19 g (88%). To a cooled (0-5° C.) solution containing N,N-bis-(diisopropylamino) chlorophosphine (415 mg, 1.55 mmol, 1.15 eq.) and triethylamine (217 microliters, 157 mg, 2.2 mmol, 1.15 eq.) in anhydrous CH2C12 (12 mL) was added dropwise a solution containing DMT-monoprotected 1,3-propanediol (512 mg, 1.35 mmol) in anhydrous CH2C12 (2 mL). The resulting mixture was stirred at r.t. under argon for 1 h (or until TLC showed the reaction to be complete, hexane-EtOAc-Et3N 70:25:5). ter-Butyldisulfide propanol (244 mg, 1.35 mmol, 1.0 eq.) in CH2C12 (1 mL) was added to the reaction mixture followed by diisopropylammonium tetrazolide (232 mg, 1.35 mmol, 1.0 eq.). The mixture was stirred at r.t. for 30 min., quenched by addition of satd. aq. NaHCO₃ (40 mL) and the phases separated. The organic layer was washed with brine (30 mL), water (50 mL), dried (MgSO4) and evaporated under diminished pressure. The residue was purified on a silica gel column (2.5×12 cm), eluting with 15:1 hexane-EtOAc containing 2% Et3N. The product was obtained as a colorless syrup: yield 494 mg (53%). Standard DNA phosphoramidites, solid supports (controlled pore) and additional reagents were purchased from Glen Research. All oligonucleotides were synthesized on an Applied Biosystems 3400 automated DNA/RNA synthesizer using a standard 1.0 μmole phosphoramidite cycle of acid-catalyzed detritylation, coupling, capping, and iodine oxidation. Stepwise coupling efficiencies and overall yields were determined by automated trityl cation conductivity monitoring. All β-cyanoethyl phosphoramidite monomers were dissolved in anhydrous acetonitrile to a concentration of 0.1M immediately prior to use. Cleavage of the oligonucleotides from the solid support and deprotection was achieved by exposure to concentrated aqueous ammonia solution for 60 min at room temperature followed by heating in a sealed tube for 4 h at 65° C. Then the synthesized strand was purified by a reversed phase HPLC.

### Cell Culture.

HeLa cells (ATCC) were cultured in Eagle's minimum essential medium (EMEM) supplemented with 10% fetal bovine serum (FBS) (ATCC) and 1% penicillin-streptomycin solution. HeLa cells were cultured at 37° C. under a humidified atmosphere of 55% CO2 and 90% humidity.

### Confocal Laser Scanning Microscopy

HeLa cells were seeded at 8×104 cells/ml on μ-Slide 18 Well (Ibidi, USA) and cultured for 24 hours after seeding. After cell confluency became around 80%, the cells were incubated with 3 nM with or without disulfide modified DNA nanostructures for 2 hours. Then the media was removed, and the cells were wash with PBS for two times. 2 ug/ml Hoechst 33342 (ThermoFisher/Invitrogen) in cell complete medium was added to the cells. After 25 min incubation at 37° C. and subsequent wash with PBS, the cells were stained with 100 nM Lysotracker Red DND-99 (ThermoFisher, Life Technologies) for 2 hours. Then imaged by Nikon C2 Laser Scanning Confocal.

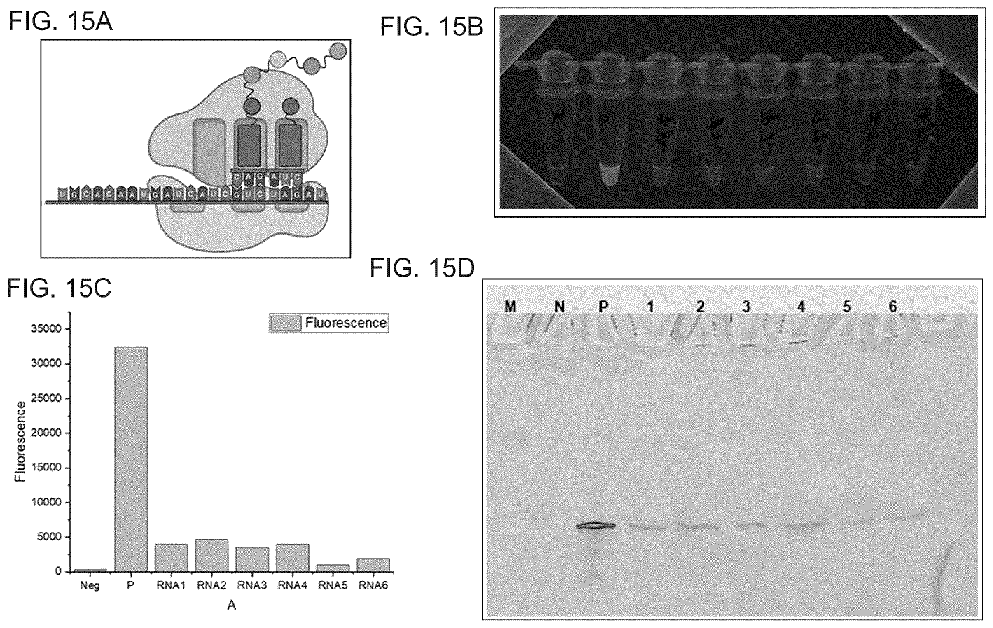

To demonstrate that the loading capacity of DNA nanostructure can be controlled, selected amounts of disulfide and FAM modified DNA strands (0, 26, 51, 101 and 189) and DNA strands without disulfide as a control were loaded into the DNA nanostructure (FIG. 15A). Each DNA nanostructure was labeled with 4 Alexa Fluor™ 647 fluorophores to normalize the fluorescence result. Comparison of the relative fluorescence intensity of samples to expected results enabled evaluation of the amount of DNA strands loaded into a DNA nanodevice. As shown in the FIG. 15, DNA nanostructures with or without disulfide modification had similar fluorescence intensity compared with the expected result under different amounts of loading strands which indicates that the amounts of loading DNA strands of DNA nanostructures can be quantitatively controlled. The stability of 104 6-disulfide modified DNA nanostructures was tested in HeLa cell complete medium which contains 10% FBS at 37° C. with varied incubation times (0, 10 min, 30 min, 1 h, 2h, 4 h, 9h, 12 h, 24 h and 52h). FIG. 15B shows that disulfide modified DNA nanostructure can remain relatively intact for at least 24 hours.

### Steady-State Spectroscopy

The steady-state fluorescence emission and excitation spectra were conducted on a Nanolog fluorometer (Horiba Jobin Yvon). Fluorescence emission was collected at 90° to the excitation light using a 1.5×1.5 mm quartz cuvette (Hellma, on al 4 sides).

### Agarose Gel Electrophoresis

Agarose gel electrophoresis (1%, 1 × TAE-Mg buffer (40 mM Tris, 20 mM acetic acid, pH 8.0, 2 mM EDTA, 12.5 mM Mg (CH₃COOH)₂) (magnesium acetate) carried at 100V in an ice-water bath. After running for 2.5 h, the gel was stained with SYBR green nucleic acid gel stain solution (Life Technologies) and imaged with Gel Dox XR+ system gel imager (Bio-Rad) with UV light excitation and standard UV filter (amber filter with the wavelength range of 548-630 nm).

### Example 4. Loading shRNA and Disulfide Modified Strands onto Closed DNA Nanotube Structures

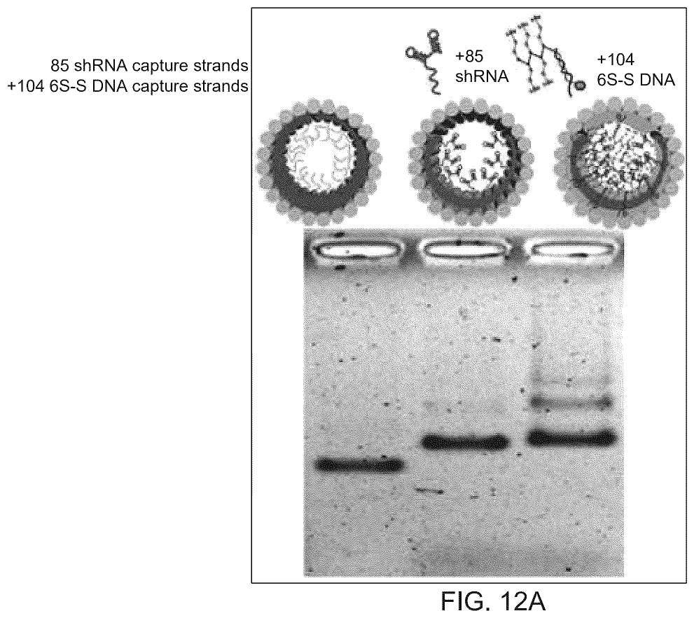

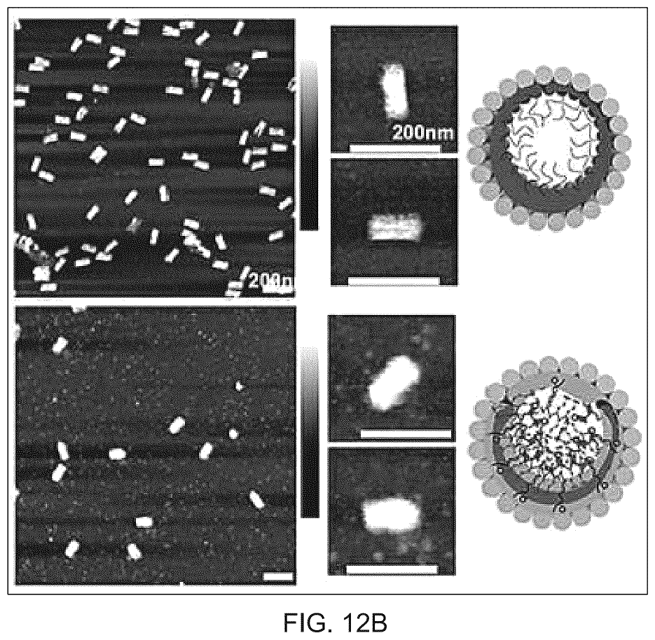

The loading of shRNA and disulfide modified strands onto closed DNA nanotube structures was established be agarose gel electrophoresis and AFM measurements. As shown in FIG. 12, Discrete bands with expected mobility were observed for closed DNA nanotube with 189 capture strands (85 for shRNA loading and 104 for disulfide modified strand loading), closed DNA nanotubes loaded with 85 shRNA and closed DNA nanotubes with both 85 shRNA and 104 disulfide modified strands. To maximize shRNA loading, first a closed DNA nanotube was prepared, then the DNA nanotube was loaded with shRNA, then loaded with a disulfide modified strand as the last loading element because the tert-butyl group of the disulfide unit will increase the hydrophobic effect of nanotube which would otherwise hinder the loading of shRNA. AFM results also unambiguously confirmed the formation of each DNA nanostructure (e.g., nanotube) with expected size and shapes.

Agarose gel electrophoresis. Agarose gel electrophoresis (1%, 1 × TAE-Mg buffer (40 mM Tris, 20 mM acetic acid, pH 8.0, 2 mM EDTA, 12.5 mM Mg (CH₃COOH)₂) was carried at 100V in an ice-water bath. After running for 2.5 h, the gel was stained with SYBR green nucleic acid gel stain solution (Life Technologies) and imaged with Gel Dox XR+ system gel imager (Bio-Rad) with UV light excitation and standard UV filter (amber filter with the wavelength range of 548-630 nm).

AFM imaging. 7 μl sample was deposited onto a fresh mica surface (Ted Pella) and let for binding for 3 min. 60 μl 1 × TAE-Mg buffer and 3 μl 0.2M NiCl₂ were then added on the top to stabilize the sample-substrate binding. The imaging was performed using Veeco 5 Multimode AFM in liquid mode with Scanasyst-fluid+ tip (Brucker).

### Example 5. RNAi Effects of shRNA Loaded on DNA Nanostructures

Western blot experiments were performed to demonstrate the RNA interference effects of shRNA loaded onto DNA nanostrures (FIG. 13). The shRNA sequences are SEQ ID NOs: 13-17. At concentration of 1.5 nM, disulfide modified DNA showed a higher silencing effect than DNA nanostructures without disulfide modification. However, when further increase the concentration of DNA nanostructure to 3 nM, there is no obvious difference between these two groups. This might because of some of the disulfide modified DNA nanostructure cannot be readily released from cell membrane. It is also possible that after disulfide modified DNA nanostructure translocated to the cytoplasm, the hydrophobic effect formed by the disulfide protection group hinders the release of shRNA. Compared with DNA nanostructure delivery system, although shRNA transfected by lipofectamine showed enhanced protein inhibition effect. However, most of the cells were dead due to the toxicity of lipofectamine, which further proved by the reduced expressed of Mcl-1 in scramble sequence transfected by lipofectamine.

Western blot. HeLa cells were treated with different groups listed in FIG. 13 in 24-well plate. After 48 hours, HeLa cells were lysed by cell lysis buffer (Sigma-Aldrich). Shake to homogenize in ice water bath for 10 min. Homogenized cells were then collected to Eppendorf tubes. Protein concentration was next quantified by a standard BCA protocol. Protein was loaded into each well of 4-20% Mini-PROTEAN amp reg (Bio-Rad) SDS-PAGE gel, electrophoresed at constant voltage 200V for 30 min to achieve optimal band separation, and transferred to membrane at constant voltage 20V for 30 min in semi-dry transfer cell (Bio-Rad). Western blots were blocked in blocking buffer (Bio-Rad) for 10 min, washed three times in 1 × TBST for 5 min each, and incubated with Mcl-1 primary antibody (rabbit monoclonal, Cell signal) overnight at 4° C. Signals were detected with secondary antibodies conjugated to horseradish peroxidase and visualized with the Renaissance Plus reagent (Santa Cruz).

### Example 6. Demonstration of shRNAs Against Mcl-1 and Bcl-Xl Induce HCC Cell Death

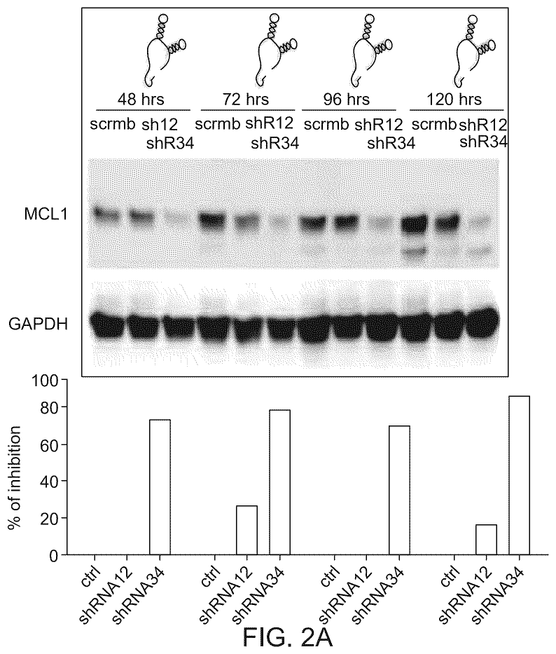

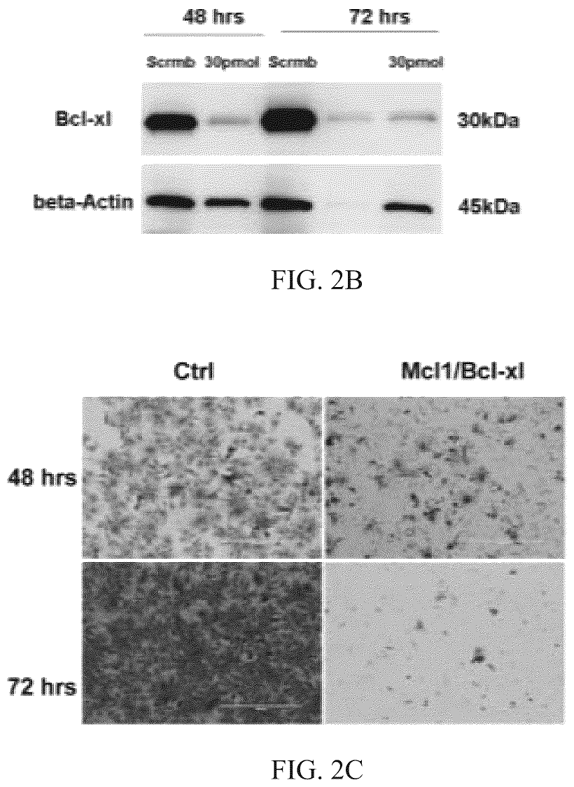

In some embodiments, the Nucleic acid nanodevices of this disclosure deliver shRNAs to within a cell, and the shRNAs exhibit a selective cytotoxic effect. To demonstrate proof of concept of downregulating mcl-1 and bcl-xl genes using the shRNAs of this disclosure, Hep3B liver cancer cells were transfected with hetero-double shRNAs against the mcl-1 and bcl-xl genes, respectively. Specifically, four positions on the mRNA were selected as the shRNA targets, and two of them were connected as the hetero-double shRNA (shR12 and shR34) and synthesized via in vitro transcription. The knockdown of the two target proteins was evaluated by western blot (FIG. 2A, 2B). Although the scramble (“scrmb”) siRNA sequence failed to exhibit any gene knockdown, shRNAs against Mcl-1 and Bcl-xl dramatically reduced the Mcl-1 and Bcl-xl protein expression levels (SEQ ID NOs: 13-17). Mcl-1 protein expression was downregulated over 80% with shRNA34 even after 120 hours, indicating that a representative embodiment of a designed hetero-double shRNA of this disclosure exhibited high and prolonged gene knockdown efficiency. The combinatorial shRNA transfection against both Mcl-1 and Bcl-xl was performed (FIG. 2C) to test the impact in cell survival after knockdown both Mcl-1 and Bcl-xl. The shRNA combination significantly induced cancer cell death as shown in FIG. 2C with the attached survival cells stained by the purple crystal blue. The results indicate that the simultaneous knockdown of Mcl-1 and Bcl-xl induces cancer cell death.

### Peptidase Activity of ADAM10 in HCC Cells.

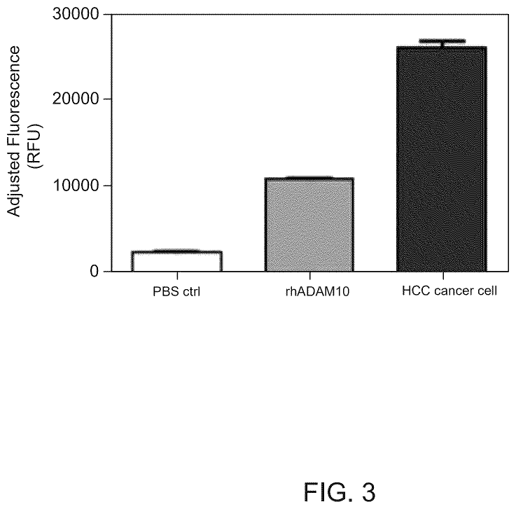

A fluorescent assay using the fluorogenic peptide substrate to ADAM10 was used to demonstrate the presence of the ADAM10 on HepG2 cell surface. This fluorogenic peptide comprises a highly fluorescent 7-methoxycounmarin group (Mca) and a 2,4-dinitrophenyl group, where the Mca group is efficiently quenched by the Dpa group. Upon cleavage by ADAM10 at the amide bond between Mca and Dpa, the peptide exhibits a strong fluorescence. As shown in FIG. 3, after incubation with HCC cells, the fluorogenic peptide significantly increased the fluorescence intensity, stronger than the recombinant human ADAM10 (rhADAM10) which serves as a positive control. This proof of concept demonstrates that using the ADAM10 substrate can be used to gate cleavage, and thus opening, of a Nucleic acid nanodevice wherein the nanodevice is in the shape of a sheet (“DNA nanosheet”) when the DNA nanosheet is brought into a closed tubular formation by locking opposite end of the DNA nanosheet with opposing ends of the ADAM10 substrate, as shown in FIG. 1B.

### Example 7. Demonstration that Enhancer Sequences can Increase Translated RNA Protein

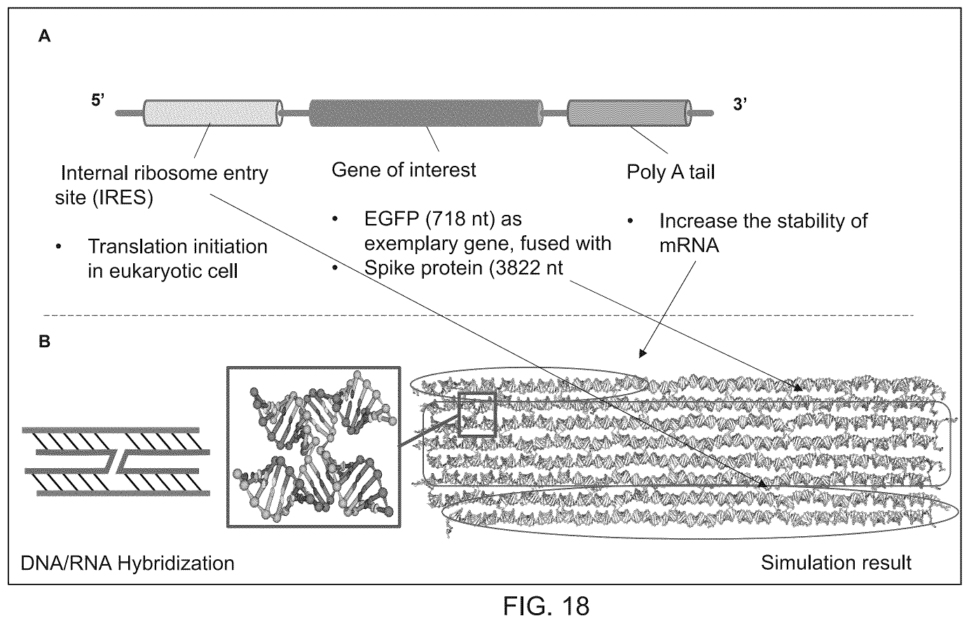

In accordance with the methods described herein for the Pro-mRNA delivery, an enhancer sequence (SEQ ID NO: 18) was made to be contiguous with the mRNA sequence of a gene encoding fluorescent protein, GFP (SEQ ID NO: 274). The double strand DNA were amplified by PCR using T7promoter/terminal primers, which contain T7 promoter and T7 terminal. Double stranded DNA was purified by a PCR clean-up kit. The RNA was transcribed with the same approach for the RNA nanostructure synthesis as described in the previous publication. As shown in FIG. 18, a large amount of PCR products and transcriptional RNA were produced by an unique high yield T7 polymerase. These results suggest that the amplification and RNA transcription approaches can produce a large amount of RNA, which is desirable for the Pro-mRNA composition.

To evaluate the protein expression by synthesizing mRNA with enhancer, the protein expression was identified by SDS-PAGE gel, fluorophore imaging, and fluorophore plate reader. For example, the green fluorophore protein gene (GFP) was used as a representative template plus enhancer sequencing and 30 polyA. The Protein in vitro expression system was used, which is a 1-Step Human Coupled IVT Kit (Invitrogen, Cat, 8882), to identify the protein expression of RNA. As shown in FIG. 11, the Enhancer sequence (“P”) increases the GFP protein expression level. The GFP protein band was found in SDS-PAGE gel compared with positive control. Without enhancer sequence, the RNA didn't express protein as negative control (as shown in “N” lane). This result indicates that enhancer sequence increases the protein expression of RNA translation process.

### Example 8. Lipid Nanoparticle (LNP) Synthesis and Characterization

Lipid nanoparticles were synthesized using the spontaneous vesicle formation method. A solution of ionizable lipid (DLin MC3-DMA), 1,2-Distearoyl-sn-glycero-3-phosphorylcholine (DSPC), 1,2-distearoyl-sn-glycero-3-phosphoethanolamine-polyenthylene glycol 2000 (PEG-DSPE-2000), and cholesterol in ethanol was prepared in a 50:10:5:35 molar ratio, respectively, and incubated at 37° C. for 5 min. A solution of oligonucleotide cargo was prepared in aqueous citrate buffer (20 mM, pH 4-5) and incubated at 37° C. for 5 min. The concentration of oligonucleotide in buffer was used based on N/P ratio as described in the following formula.

$$\frac{N}{P} = \frac{Number of moles of ionizable lipid}{Number of moles of oligonucleotide cargo \times number of charge on phosphate bacbone}$$

The N/P ratio was selected to be in the range of in 1-4 for minimal cytotoxicity and efficient endosomal escape. Then lipid mixture was slowly mixed with oligonucleotide aqueous solution for 30 sec and incubated at room temperature for 60 min. The final lipid concentration was kept 15 mM in ethanol: water (1:1.25) solution. Thereafter formed oligonucleotide-lipid self-assembly mixture was dialyzed using 10 kDa dialyzer against citrate buffer (20 mM, pH 4-5) for 2 h at room temperature to remove ethanol. This mixture was finally transferred into 100 kDa dialyzer and dialyzed against 1Xphosphate buffered saline (PBS) for 12 h at 4° C.

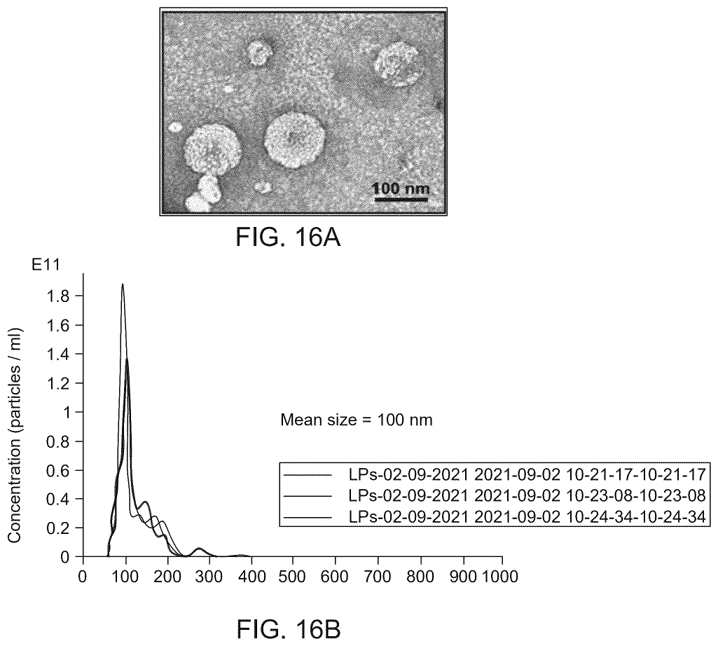

Prepared lipid nanoparticles-oligonucleotide self-assembly was characterized by negative stain transmission electron microscopy (TEM) in FIG. 16A; and nanoparticle tracking analysis (NTA) as shown in FIG. 16B, which indicated that the particles were consistently about 100 nm in diameter.

### Example 9. Small RNA Carriers for Cell Delivery

The Aspartate beta-hydroxylase (ASPH), a type II transmembrane protein, is highly expressed in HCC, as well as in other tumors, such as breast, lung, colon, pancreatic, ovarian, and brain tumors. ASPH is a cell-surface enzyme that generates enhanced cell motility, proliferation, invasion, and metastasis in HCC. ASPH overexpression predicts a poor prognosis with early disease reoccurrence and worse clinical outcomes of patients in HCC. Several small molecule inhibitors, as well as antibodies against ASPH are known to suppress tumor proliferation and metastasis in vitro and in vivo. It is, therefore, a well-recognized therapeutic target in cancer treatment. Therefore, the ASPH antibody can be used as a target to deliver small RNA carriers of this disclosure to the tumor site. A Disintegrant and Metalloprotease Domain 10 (ADAM10) protein is a membrane-bound peptidase that cleaves extracellular portions of transmembrane proteins and was discovered to interact physically with ASPH. ADAM10 is a transmembrane protease that is important for embryonic development and tissue homeostasis. Employing the peptidase activity of ADAM10, its substrate peptide will be used as the lock of the small RNA carrier comprising a DNA nanostructure in the form of a closed tube. After targeting tumor cells via ASPH antibodies, the DNA nanostructure will open up, exposing multi-disulfide units, which enables rapid cytosolic internalization in response to the peptide cleavage by cell surface ADAM10 protein (FIG. 1).

Design and Development of a Modular RNA Carrier System for Specific Delivery Of shRNAs/p-mRNA into Liver Tumor Cells.

Described herein is a hetero double shRNA and nucleic acid nanodevice (wherein the nucleic acid is DNA) that can be loaded with multiple functional molecules for tumor cell targeting and killing. Hetero-double shRNA can recognize 2 positions of gene mRNA to silence the gene with high efficiency. A reconfiguration of DNA nanodevice occurs with peptide lock cleaved by a membrane protein, ADAM10, upon tumor cell targeting with ASPH antibodies. Consequently, the rapid cytosolic delivery of shRNAs/Pro-mRNA cargos will be able to occur via thiol-mediated internalization (FIG. 1). The results are expected to demonstrate the DNA nanodevice can be updaten by cells when said nucleic acid nanodevice comprises disulfide-unit modifications.

### Design and Characterization of Nucleic Acid Nanodevice for Tumor Cell Targeting.

First, the ASPH antibody will be conjugated to the Nucleic acid nanodevice for a cell targeting system. The antibody-DNA conjugates will be prepared using sulfo-SMCC as a bifunctional crosslinker between ASPH antibody and a DNA oligonucleotide, as described previously. The antibody-DNA conjugates will then be loaded onto the open Nucleic acid nanodevice through DNA hybridization and characterized by atomic force microscopy (AFM). The tumor cell targeting assay will be performed with antibody-loaded AF647-Nucleic acid nanodevice through confocal fluorescence microscopy and flow cytometry analysis. The AF647-Nucleic acid nanodevice without ASPH antibody loading, as well as with a random antibody loading, will be served as negative controls. The number of ASPH antibodies conjugated on the Nucleic acid nanodevice will be tuned to achieve optimal tumor cell targeting efficiency.

### Design and Characterization of Nucleic Acid Nanodevice Opening

The ADAM10 substrate peptide will be employed as the lock to close the Nucleic acid nanodevice (where the nucleic acid is DNA). Peptide-DNA conjugates will be created via click chemistry reactions between DBCO-conjugated DNA oligonucleotides and azido-lysine-containing peptides. Upon hybridization between DNA oligonucleotides and capture strands on the Nucleic acid nanodevice, the closed Nucleic acid nanodevice will be formed (FIG. 8). The closing and opening of the DNA nanodevice will be characterized by AFM before and after incubation with the rhADAM10 protein. In addition, the same fluorescent assay will be performed by incubating closed DNA nanodevice with HCC cells (FIG. 3). Furthermore, the number of peptide locks will be tuned for efficient closing of the DNA nanodevice as well as enabling the rapid opening of the DNA nanodevice upon incubation with HCC cells.

Optimization of Disulfide-Unit and shRNA Loading.

The DNA nanostructure potentially allows loading up to 200 functional molecules, a high capacity for loading enough disulfide-unit to mediate internalization and shRNAs/p-mRNA as drugs. The number and distribution of disulfide-unit conjugated on the DNA nanostructure will first be determined. The minimum amount of the disulfide unit, which allows comparable internalization efficiency, will eventually be employed for the final design. This enables the maximum loading capacity for the shRNA cargos to achieve the best tumor-killing effect. The FAM-DNA nanostructure loaded with various amounts of disulfide-unit will be utilized for cell internalization assay. The cytosolic internalization efficiency will be quantitated using flow cytometry analysis, confocal microscopy, and cell surface plasmon resonance (SPR). The endosome internalized DNA nanostructure with a small amount of disulfide-unit will unlikely be analyzed in this approach as FAM is not fluorescent under the endosome pH as indicated in FIG. 5. Confocal fluorescent microscopy will be used to confirm the cytosolic internalization with the minimum disulfide-unit conjugated DNA nanostructure.

### In Vitro Assessment of the Functionalized Nucleic Acid Nanodevice.

After validating the construction and modification of the Nucleic acid nanodevice for targeting and opening with HCC cell lines, in vitro characterization will be performed to assess its functionality. The shRNAs and disulfide-unit will be loaded inside of the Nucleic acid nanodevice with the similar strategy described in FIG. 5. While ASPH antibodies are loaded on the outside portion (FIG. 2). After closed Nucleic acid nanodevice is formed by peptide lock, the cytosolic internalization of the full Nucleic acid nanodevice will be carried out using confocal fluorescence microscopy. Characterization of RNAi mediated gene knockdown will be performed with RT-PCR for mRNA assay and western blot analysis of the Bcl-xl and Mell proteins as described in FIG. 4. RNAi mediated tumor cell apoptosis will also be analyzed via flowcytometry and confocal microscopy. The ratio of the disulfide-unit and shRNAs loaded inside the closed Nucleic acid nanodevice will be further tuned to achieve optimal delivery efficiency as well as gene knockdown efficacy.

Expected outcomes, potential pitfalls, and alternative approaches: the results are expected to demonstrate that shRNAs can be delivered into the cytoplasm of HCC tumor cells for apoptosis and demonstrate the following actions: 1. Targeting of Nucleic acid nanodevice to tumor cells; 2. Controlled opening and consequent cytosolic internalization of Nucleic acid nanodevice; 3. shRNAs/p-mRNA mediated tumor cell apoptosis. The antibody conjugation is random and has the potential to target multiple lysine positions on the surface which would lead the antibody-DNA conjugates to exhibit decreased binding affinity with ASPH protein. Multiple copies of the antibodies will be loaded onto the Nucleic acid nanodevice. If the peptide lock is not efficiently recognized and cleaved by the ADAM10 protein on the cell surface due to a possible steric hindrance, longer DNA and/or peptide linkers flanking the peptide lock can be introduced to allow the efficient recognition and cleavage by cell surface ADAM10 protein.

### In Vivo Test of the Multi-Functional Nucleic Acid Nanodevice for HCC Therapeutics In Xenograft Mice Liver Tumor Models.

The nucleic acid nanodevices described herein can exhibit a tumor-killing effect as will be demonstrated in xenograft mice liver tumor models generated by human liver tumor cell lines, HepG2 and Hep3B. DNA nanodevices can be programmed for targeted delivery to tumor blood vessels and subsequently induce tumor infarction. This approach led to significant delays in tumor growth but caused no apparent harm to normal blood vessels. This same strategy will be employed with the multi-functional Nucleic acid nanodevice described herein for demonstrating the direct tumor killing in vivo. Compositions comprising a series of DNA or RNA nanostructures described herein will be intravenously delivered to a mouse xenograph model, and the results monitored by established fluorescence-based monitoring tools, to screen for the most potent multi-functional DNA nanostructures with good functional activity but low adversity.

Stable mRNA Nanostructures to Resist RNAse Degradation

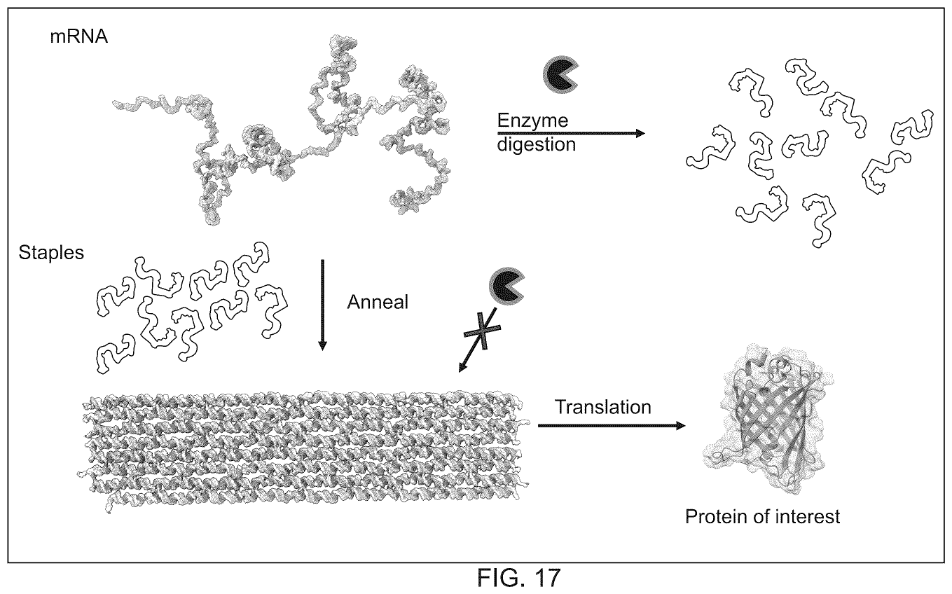

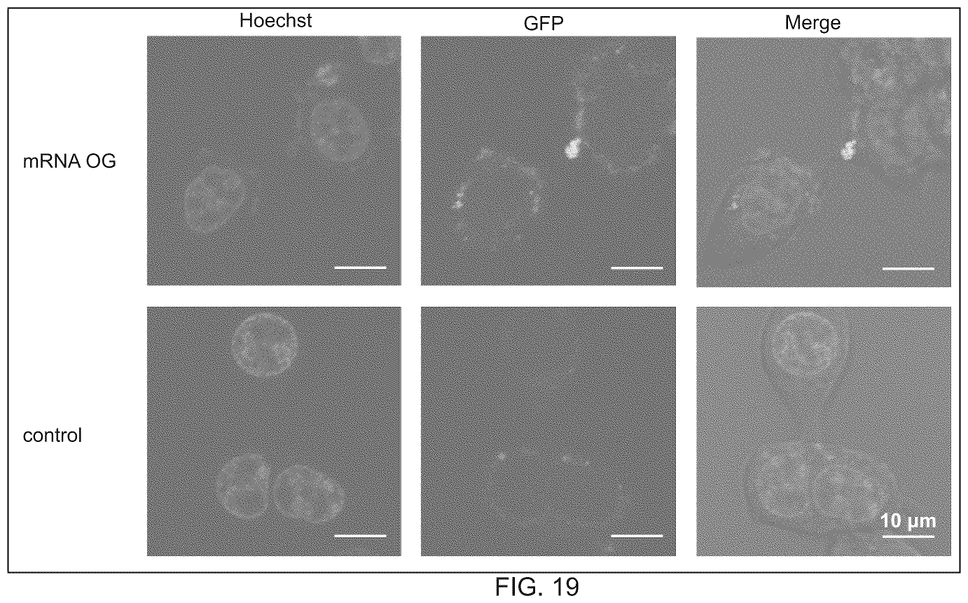

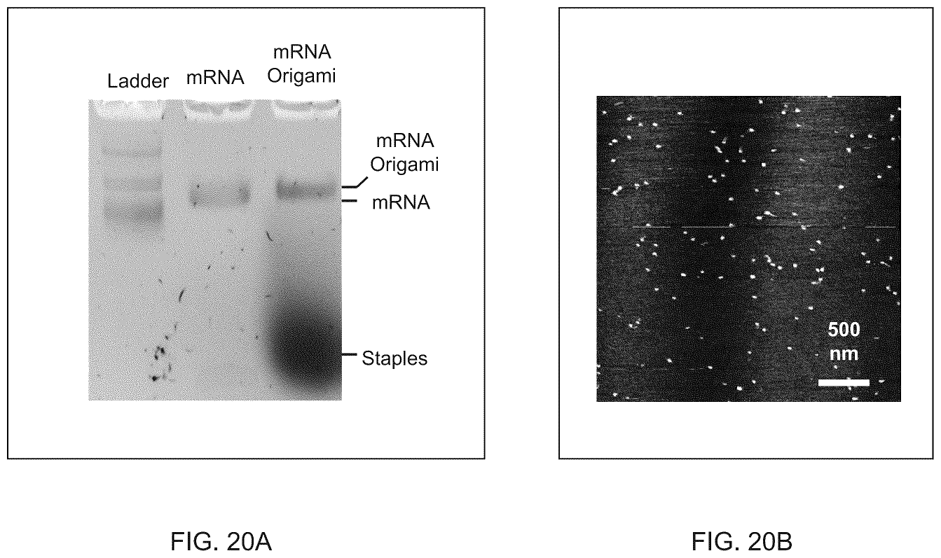

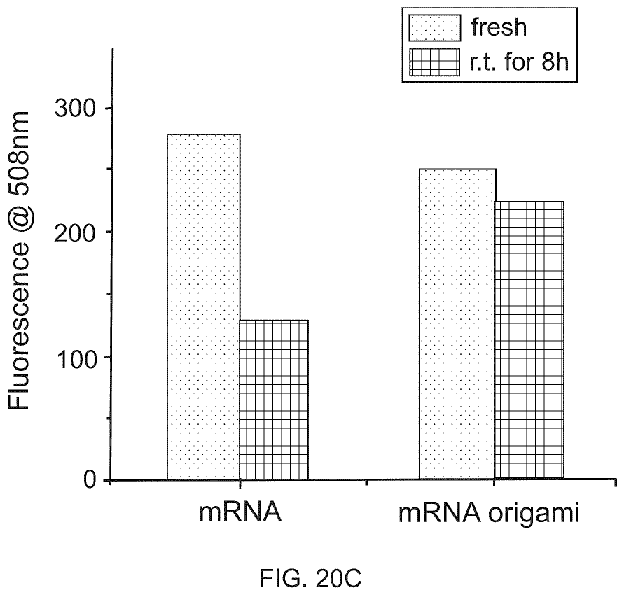

Informational mRNA (e.g., to make mRNA-based vaccines) can be stabilized to cellular environmental conditions, by forming “programmed mRNA” to enhance the stability of these vaccines. The core concept as illustrated in FIG. 17 involves folded mRNA into compact nanostructures to resist digestion from RNase with the assistance of short DNA staple strand oligonucleotides. More stable mRNA results in a higher amount of mRNA presented to the cell, thereby increasing the efficiency of translation for mRNA. The mRNA molecule is designed with three fragments: an internal ribosome entry site (IRES) for the cap-free translation initiation, an open reading frame for the translation of the target protein, (e.g. green fluorescence protein (GFP)), and a 3′ polyA tail for mRNA stability. The mRNA strand and 31 short DNA staple strands are designed into a rectangular-shape origami by Cadnano, with 11 bp as the helix pitch (FIG. 18). The mRNA strand is synthesized, then annealed with DNA staple strand oligonucleotides. Both agarose gel electrophoresis and atomic force microscopy (AFM) results confirm the formation of mRNA nanostructures. In vitro translation experiments demonstrate that the folded mRNA nanostructures can express GFP comparably to regular mRNA. The synthesized mRNA nanostructure is delivered into cell via Lipofectamine to test its translation efficiency. The confocal microscopy result indicates that the folded mRNA origami can be translated into GFP in the cell (FIG. 19). Storage at room temperature for 8 hours only slightly reduces expression efficiency, whereas the efficiency of conventional mRNA declines dramatically (FIG. 20).

### Targeted Delivery of Modular RNA Carrier System to Primary Tumors.

To monitor in vivo delivery of an embodiment of a modular RNA carrier system, the iRFP-tumor model will be used with a Cy7-labeled modular RNA carrier system so that the modular RNA carrier system distribution and tumor growth can be followed simultaneously in real-time. The lentivirus containing iRFP reporter gene will be employed to transduce HepG2 and Hep3B tumor cells to generate stable cell lines expression iRFP. Cy7 dye can be readily conjugated onto a DNA or RNA nanostructure through click chemistry reaction using Cy7 NHS ester and amine-modified DNA or RNA. The speed at which the modular RNA carrier system reaches to the tumor site will be measured, and also how long they are retained there in comparison to their distribution in the rest of the body. This information will be useful for assessing efficacy and toxicity. Nucleoli aptamers conjugated to a DNA nanocage exhibit tumor localization. By similar logic, the inventors have developed a modular RNA carrier system which is expected to not only localize to the tumor site but will also be retained in the tumor tissue for longer period of time, as internalization will occur in the tumor cells. The tumor delivery efficiency for the modular RNA carrier system will be evaluated for both with and without the ASPH antibodies. The results are expected to demonstrate that inclusion of the ASPH antibodies in the modular RNA carrier system will significantly increase tumor toxicity.

### Efficacy in the Tumor-Directed Killing of Modular RNA Carrier System.

The functional activity of a representative modular RNA carrier system will be evaluated after intravenous delivery. After confirmation that the modular RNA carrier system can be efficiently delivered to the tumor sites following injection, its impact on tumor growth will also be measured. The iRFP-tumor growth will be monitored and quantified using LI-COR Pearl Imager. After euthanizing mice, the tumor tissue slices will be prepared for H/E, IHC staining. Tumor cells will also be harvested for western blot analysis to determine the gene knockdown efficiency. To assess the toxicity of the modular RNA carrier system, the procedure for analyzing thrombin-loaded DNA nanocages will also be employed. DNA nanocages loaded with multiple functional groups will be measured as to any elevated pro-inflammatory responses. The mice serum will be taken after modular RNA carrier system injection for measurements with cytokine arrays.

The results are expected to demonstrate that the modular RNA carrier system involving a DNA nanocage allows for targeting, controlled opening, and internalization at the tumor site. Furthermore, the modular RNA carrier system-based RNAi therapy is expected to inhibit tumor growth by direct killing tumor cells. The success of the tumor-infarction DNA nanocage in delaying tumor growth in a mouse model, suggests that the modular RNA carrier system described herein are expected to have sufficient stabilities to exert its intended functions. Initial results indicated that the gene knockdown triggered by shRNAs lasted for at least 5 days. However, if the stability of unmodified shRNAs is an issue in the in vivo test, the chemically modified siRNA/shRNA (e.g. 2′-F, 2′-OMe, and phosphonothioate backbone) can be employed for prolonged in vivo half-lives. Phosphonothioate bond modification could also be employed to the DNA linker region of the peptide lock to further enhance the overall stability of the modular RNA carrier system. If tumor site accumulation of modular RNA carrier system is poor under the intravenous delivery method, intratumor delivery will be employed.

All DNA and RNA sequences presented herein are oriented 5′->3′, unless noted otherwise.

Although the foregoing specification and examples fully disclose and enable certain embodiments, they are not intended to limit the scope, which is defined by the claims appended hereto.

All publications, patents and patent applications are incorporated herein by reference. While in the foregoing specification certain embodiments have been described, and many details have been set forth for purposes of illustration, it will be apparent to those skilled in the art that additional embodiments and certain details described herein may be varied considerably without departing from basic principles.

The use of the terms “a” and “an” and “the” and similar referents are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the technology and does not pose a limitation on the scope of the technology unless otherwise claimed. No language in the specification should be construed as indicating any non-claimed element as essential to the practice of the technology.

Throughout this specification, unless the context requires otherwise, the word “comprise” or variations such as “comprises” or “comprising”, will be understood to imply the inclusion of a stated integer or group of integers but not the exclusion of any other integer or group of integers. It is also noted that in this disclosure and particularly in the claims and/or paragraphs, terms such as “comprises”, “comprised”, “comprising” and the like; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the embodiment.

Embodiments are described herein, including the best mode known to the inventors. Variations of those embodiments may become apparent to those of ordinary skill in the art upon reading the foregoing description. The inventors expect skilled artisans to employ such variations as appropriate, and the inventors intend for the embodiments to be practiced otherwise than as specifically described herein. Accordingly, this technology includes all modifications and equivalents of the subject matter recited in the claims appended hereto as permitted by applicable law. Moreover, any combination of the above-described elements in all possible variations thereof is encompassed by embodiments unless otherwise indicated herein or otherwise clearly contradicted by context.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A multivalent RNA oligonucleotide junction delivery complex comprising:
a. four core RNA oligonucleotides comprising:
i. a first RNA oligonucleotide,
ii. a second RNA oligonucleotide,
iii. a third RNA oligonucleotide, and
iv. a fourth RNA oligonucleotide,
wherein a portion of the first RNA oligonucleotide is partially complementary to the second RNA oligonucleotide to form a first arm, and also partially complementary to the fourth RNA oligonucleotide to form a fourth arm;
wherein a portion of the second RNA oligonucleotide is partially complementary to the third RNA oligonucleotide to form a second arm, and is also partially complementary to the first RNA oligonucleotide to form the first arm;
wherein a portion of the third RNA oligonucleotide is partially complementary to the fourth RNA oligonucleotide to form the third arm and also partially complementary to the second RNA oligonucleotide to form the second arm;
and wherein a portion of the fourth RNA oligonucleotide is partially complementary to the first RNA oligonucleotide to form the fourth arm, and is partially complementary to the third RNA oligonucleotide to form the third arm;
b. four peripheral RNA oligonucleotides comprising:
i. a first peripheral oligonucleotide;
ii. a second peripheral oligonucleotide;
iii. a third peripheral oligonucleotide; and
iv. a fourth peripheral oligonucleotide,
wherein the first, second, third, and fourth peripheral oligonucleotides independently comprise a trigger;
wherein the first second, third, and fourth peripheral oligonucleotides optionally independently comprise two separate discontinuous nucleic acid sequences;
wherein the first, second, third, and fourth peripheral oligonucleotides are partially or wholly complementary to the first RNA oligonucleotide, second RNA oligonucleotide, third RNA oligonucleotide, or fourth RNA oligonucleotides; and
wherein the trigger is selected from a disulfide-modified sequence, a targeting molecule, an enhancer sequence, a programmed mRNA (Pro-mRNA) sequence, or combinations thereof.

2. The complex of claim 1, wherein the enhancer sequence comprises a G-gap independent Internal ribosome entry site (IRES).

3. The complex of claim 1, wherein the enhancer sequence comprises a recruiter to a RNA binding protein sequence, wherein the RNA binding protein comprises the eukaryotic initiation factor 3 (elF3) complex.

4. The complex of claim 1, wherein the enhancer and Pro-mRNA are contiguous.

5. The complex of claim 1, wherein the four core RNA oligonucleotides arms form a cross-shape with each arm about at a 90 degree angle to two neighboring arms.

6. The complex of claim 1, wherein the enhancer provides for the initiation of Pro-mRNA translation.

7. The complex of claim 1, wherein the targeting molecule is selected from: GalNAc, an affibody, a shRNA, or an anti-cancer agent.

8. The complex of claim 7, wherein the affibody is an antibody or portion thereof to ASPH or Her2.

9. The complex of claim 7, wherein the shRNA is selected from: MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13).

10. The complex of claim 1, wherein the complex comprises two Pro-mRNA contiguous with RNA protein binding sites and enhancers, two disulfide-modified peripheral oligonucleotides, and two targeting molecules.

11. The complex of claim 1, wherein the Pro-mRNA encodes for a cancer neoantigen.

12. The complex of claim 11, wherein the cancer neoantigen is expressed from all or a portion of a tumor associated gene.

13. The complex of claim 12, wherein the tumor associate gene is selected from: AP2S1, Survivin, CTSL, MPZL2, and LSP1.

14. The complex of claim 1, wherein the Pro-mRNA comprises a Kozak sequence.

15. The complex of claim 14, wherein the Kozak sequence comprises the sequence AUG.

16. The complex of claim 1, wherein the Pro-mRNA comprises a polyA tail having from 25 to 35 contiguous adenosines.

17. The complex of claim 1, wherein the Pro-mRNA comprises an open reading frame (ORF).

18. The complex of claim 1, wherein the Pro-mRNA comprises a long mRNA strand and a plurality of RNA staple strands.

19. The complex of claim 18, wherein the portions of the long mRNA strand are partially complementary to each of the plurality of RNA staple strands such that the Pro-mRNA folds into a selected shape.

20. The complex of claim 19, wherein the shape of the Pro-mRNA is a flat sheet.

21. The complex of claim 1, wherein the disulfide (LD)-modified siRNA are designed as follows: 5 repeated LD units are introduced at the 5′ end of the passenger strand and the 3′ ends of the guide strand.

22. The complex of claim 21, wherein the LD unit to the 5′ end of the guide strand prevents RNA-induced silencing complex (RISC) formation.

23. A lipid nanoparticle comprising a complex of claim 1.

24. A shRNA sequence selected from: MCL-1 shRNA (SEQ ID NO: 1500), MCL-12 shRNA (SEQ ID NO: 1501), MCL-34 shRNA (SEQ ID NO: 1502), BCL-XL shR12 (SEQ ID NO: 15), BCL-XL shR13 (SEQ ID NO: 14), or BCL-XL shR34 (SEQ ID NO: 13).

25. A lipid nanoparticle comprising a shRNA sequence of claim 24.

26. A method of killing a cancer cell, the method comprising presenting the complex of claim 1 to a cancer cell,
wherein the cancer is a colorectal cancer, pancreatic cancer, breast cancer, ovarian cancer, lung cancer or melanoma.

27. A method of treating cancer in a subject in need thereof, the method comprising administering to the subject a therapeutically effective amount of a composition comprising a complex of claim 1 to a t,
wherein the cancer is a colorectal cancer, pancreatic cancer, breast cancer, ovarian cancer, lung cancer or melanoma.

28. (canceled)

29. The method of claim 27, further comprising administering to the subject a therapeutically effective amount of at least one anti-cancer agent.

30. (canceled)

31. (canceled)
